(19) United States
(12) Patent Application Publication
(10) Pub. No.: US 2006/0029586 A1
(43) Pub. Date: Feb. 9, 2006

(54) Use of isocyanate linkers to make hydrolyzable active agent biopolymer conjugates

(75) Inventors: Qingqi Chen, Novato, CA (US); Damian Sowa, Vancouver (CA); Reinhard Gabathuler, San Rafael, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BIOMARIN PHARMACEUTICAL INC.

(21) Appl. No.: 11/036,259

(22) Filed: Jan. 14, 2005

Related U.S. Application Data
(63) Continuation of application No. 10/618,405, filed on Jul. 11, 2003, now abandoned.
(60) Provisional application No. 60/395,762, filed on Jul. 12, 2002.

(51) Int. Cl. A61K 38/16; A61K 38/43; C07K 14/47; C12N 9/00

(52) U.S. Cl. 424/94.1; 530/402; 514/2; 514/8

(57) ABSTRACT

The invention provides bioconjugates of the formula: 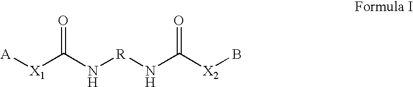
in which A is an active agent comprising an active hydroxy or amino functionality and B is a biopolymer comprising an active hydroxy or amino functionality, and X₁ and X₂ are independently N or O, and R is substituted alkyl or unsubstituted alkyl or unsubstituted or substituted heteroalkyl from 1 to about 30 atoms in length. The biopolymer may for instance be a transport protein or an antibody directed to a transport protein (e.g., a p97 protein or antibody thereto). The active agent may be a therapeutic agent such as a chemotherapeutic agent or an antineoplastic agent or an enzyme. The bioconjugates find therapeutic usages according to the therapeutic indications of the active agent. Methods of making the bioconjugates and bifunctional isocyanate cross linking reagents of use in making such bioconjugates are provided.

### CROSS-REFERENCE TO PRIORITY APPLICATION(S)

The present application claims benefit of priority of under 35 U.S.C. §119(e) U.S. Patent Application No. 60/395,762 filed Jul. 12, 2002. The entire text of aforementioned application and figures thereof is incorporated herein by reference.

### FIELD OF THE INVENTION

The field of this invention is bioconjugate pharmacology.

### BACKGROUND OF THE INVENTION

Bioconjugation of active agents to biopolymers such as proteins, polynucleic acids, and polysaccharides can provide useful substances possessing the combined properties of both the active agent and the biopolymer. For instance, conjugation of a drug to a protein or antibody can provide a therapeutic substance with an improved specificity, selectivity, affinity, or therapeutic index as compared to the free drug. Conjugation of a detectable label to a biopolymer can provide a biopolymer whose movement can be more easily monitored in vivo or in vitro. Preferably, such bioconjugates are stable to hydrolysis under physiological conditions in order to preserve the benefits of the combination.

Many reagents and cross linkers can be used to prepare bioconjugates of an active agent and a biopolymer. See, for instance, Hermanson, G T et al. Bioconjugate Techniques, Academic Press, (1996). Isocyanate cross-linking reagents can be used to cross-link biomolecules having a hydroxyl functional group to form a chemically stable carbamate link therewith or to cross link molecules having an amine functional group to form a chemically stable isourea linkage therewith. However, such isocyanate reagents are generally disfavored as they decompose rapidly in the presence of moisture.

One difficulty with such bioconjugates is the potential for the conjugated biopolymer or linking chemistry to interfere with the biological activity of the conjugated active agent. The present invention provides bioconjugates of biopolymers and active agents in which the active agents are linked via bonds which can be hydrolyzed in vivo to release the active agent. The present invention also provides synthetic methods and linking reagents for making such conjugates.

### SUMMARY OF THE INVENTION

In one aspect, the present invention provides bioconjugates of biopolymers and active agents in which the active agents are linked through isourea and carbamate linkages that are subject to enzymatic attack in vivo and in vitro. These bioconjugates are of the formula 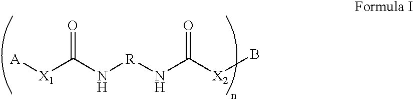
in which A is an active agent comprising an active hydroxy or amino functionality and B is a biopolymer comprising an active hydroxy or amino functionality; X₁ and X₂ are independently N or O; R is substituted alkyl or unsubstituted alkyl or unsubstituted or substituted heteroalkyl from 1 to about 30 atoms or 1-50 atoms in length, and n is from 1 to 30. In one embodiment, the biopolymer is a transport protein (e.g., one undergoing transcytosis, or one which binds and transports an entity across one or more cell membrane barriers in the body) or antibody. In one embodiment, the biopolymer comprises a mammalian or human p97 protein or fragments of a mammalian or human p97 protein. In another embodiment, the therapeutic agent is a chemotherapeutic agent or an antineoplastic agent. In another embodiment, the active agent has enzymatic activity or is an enzyme of a type deficient in the intended subject. In one embodiment, the therapeutic agent is a drug useful in treating a disorder, condition, or disease of the central nervous system or which modulates an activity within the central nervous system. In one embodiment, n is from 1 to 3 or from 1 to 10. In another embodiment, n is from 2-20. In one embodiment, the bioconjugate of Formula I is labeled. In a further embodiment, the label is a fluorescent label covalently attached to the bioconjugate through a carbamate or isourea group.

In another aspect the present invention provides methods and linking reagents for making such bioconjugates. The invention is drawn to the use of bifunctional linking reagents of Formula II and Formula III to make bioconjugates of an active agent and a biopolymer according to Formula I. The bifunctional linking reagents of Formula II and Formula III having the general formulae:

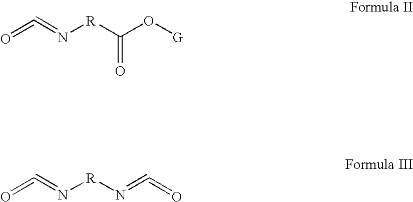

In Formula II, G represents a protecting or blocking group. In one embodiment, the blocking group is tert-butyl to form the corresponding t-butyl ester. In Formula II and Formula III, R is substituted alkyl or unsubstituted alkyl or unsubstituted or substituted heteroalkyl from 1 to about 30 atoms in length. In one embodiment, R is poly(methylene). In another embodiment, R is —(CH₂CH₂O)ₙ—, a polyethylene glycol.

In one embodiment, the bioconjugate compound of Formula I is made by contacting one of A or B with a bifunctional isocyanate compound of one of the following formulae: 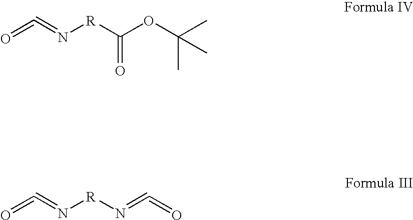
under reaction conditions wherein the isocyanate functional group covalently reacts with a hydroxy or amino group of A or B to form a first reaction product, and then the other of A or B is contacted with the first reaction product under reaction conditions wherein the first reaction product reacts with the other of A or B to form the bioconjugate compound.

In another aspect, the invention provides bioconjugates of an active agent and a biopolymer in which the active agent is a therapeutic agent and the biopolymer is a protein which transports or directs or delivers the therapeutic agent to a target site or target compartment and in which the linkage between the therapeutic agent and the biopolymer is advantageously subject to hydrolysis upon contact with enzymes (e.g., proteases, esterases, etc.,) in vivo to release the active agent at the target site or compartment. In one embodiment, the bioconjugate is administered to a subject in need of the therapeutic agent at the target site or compartment and the hydrolyzing enzyme is endogenous to the subject. In a further embodiment, the biopolymer is a transport protein or antibody. In one embodiment, the biopolymer is a p97 protein. In another embodiment, the therapeutic agent is a chemotherapeutic agent or an antineoplastic agent. In another embodiment, the active agent has an enzymatic activity or is an enzyme of a type deficient in the intended subject of administration. In one further embodiment, the therapeutic agent is a drug useful in treating a disorder, condition, or disease of the central nervous system or which modulates an activity within the central nervous system. In one such embodiment, the hydrolyzable bioconjugates are those of Formula I.

In another aspect, the invention provides pharmaceutical compositions comprising compounds according to Formula I and methods of using such pharmaceutical compositions. In one embodiment, the invention provides a pharmaceutical composition comprising a bioconjugate according to Formula I for delivering an active agent across the blood brain barrier or into an intracellular compartment comprising the active agent and a biopolymer which is p97 or a substance which is capable of specifically binding to p97. The bioconjugate can be administered in a pharmaceutically acceptable carrier or diluent. The biopolymer, preferably antibody to p97 may be conjugated to the agent. In other embodiments, a p97 fusion protein may be used as the biopolymer of the bioconjugate. The active agent may be a substance having therapeutic activity such as a growth factor or lymphokine or drug. The invention also relates to a method of delivering an active agent across the blood brain barrier comprising administering a bioconjugate of Formula I wherein the biopolymer comprises a protein undergoing transcytosis such as p97 or an antibody to such protein or p97 or a p97 protein portion or p97 fragment with p97 transport activity. The composition of the invention may also be used for delivering an agent across the blood eye or blood placenta barrier.

### BRIEF DESCRIPTION OF THE DRAWING(S)

The following drawings form part of the present specification and are included to further illustrate aspects of the present invention. The invention may be better understood by reference to the drawings in combination with the detailed description of the specific embodiments presented herein.

FIG. 1 shows the UV-Vis absorbance spectrum of 10-hydroxycamptothecin 6-isocyanatehexylcarbamate in (DMF).

FIG. 2 shows the UV-Vis absorbance, at 280 nm and 382 nm, of 10-hydroxycamptothecin in 30% DMF and 70% PBS (pH 7.4).

FIG. 3-FIG. 8 show exemplary FPLC traces of 10-hydroxycamptothecin-p97 conjugate at various reaction times. Reaction condition: DMF=30%, compound 450 equivalent, r.t., pH=7.40. Column: BioSep 300 size exclusive, buffer: 0.1M sodium phosphate, pH 6.8. The peak at 8.20 min is assigned to starting p97 and 6.95 min for the expected 10-hydroxycamptothecin conjugate. FIG. 3 shows an FPLC trace from a 1 hour reaction time. FIG. 4 shows an FPLC trace from a 2 hours reaction time. FIG. 5 shows an FPLC trace from a 4 hours reaction time. FIG. 6 shows an FPLC trace from a 6 hours reaction time. FIG. 7 shows an FPLC trace from a 9 hours reaction time. FIG. 8 shows an FPLC trace from a 20 hours reaction time. The FPLC conditions used to obtain these FPLC profiles were:

Variables

| Column | 7 | {ml} |
|---|---|---|
| Wavelength_1 | 280 | {nm} |
| Wavelength_2 | 382 | {nm} |
| Wavelength_3 | 420 | {nm} |
| Pressure_Limit | 5 | {MPa} |
| Averaging_Time_UV | 5.12 | {sec} |
| Flow_rate | 1.00 | {ml/min} |
| Equilibrate_with | 0.5 | {CV} |
| Empty_loop_with | 0.5 | {ml} |
| Eluate_Frac_Size | 0.000 | {ml} |
| Start_Eluate_Frac_at | NextTube | |
| Peak_Frac_Size | 0.000 | {ml} |
| Start_Peak_Frac_at | NextTube | |
| Peak_Start_Slope | 100.00 | {mAU/min} |
| Peak_End_Slope | 75.00 | {mAU/min} |
| Minimum_Peak_Width | 0.15 | {min} |
| Length_of_elution | 5 | {CV} |

Scouting
Questions

| | |
|---|---|
| No 1: | Sample volume and type: |
| No 2: | Column: |
| No 3: | Eluent: |

FIG. 9 shows peak area ratio and peak height ratio (280 nm/382 nm) of conjugate 5 with reaction time (h). (See table 2). Reaction condition: DMF=30%, compound 450 equivalent, r.t., pH=7.40. FPLC: Column: BioSep 300 size exclusive, buffer: 0.1M sodium phosphate, pH 6.8, flow rate: 1 mL/min.

FIG. 10-FIG. 13 show the effect of different molar equivalent excess to the MSR.

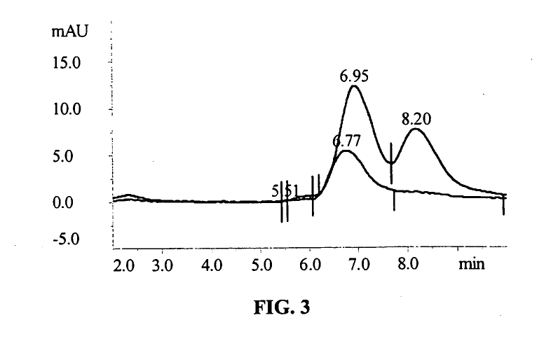

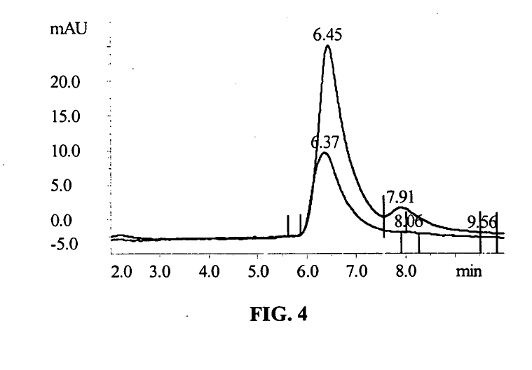

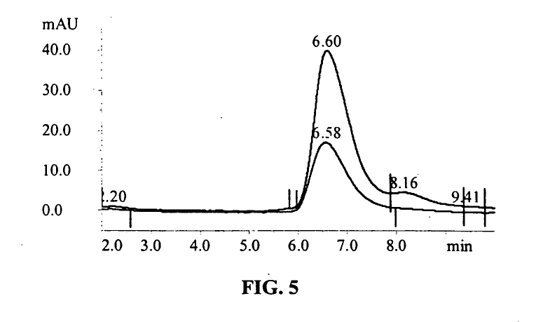

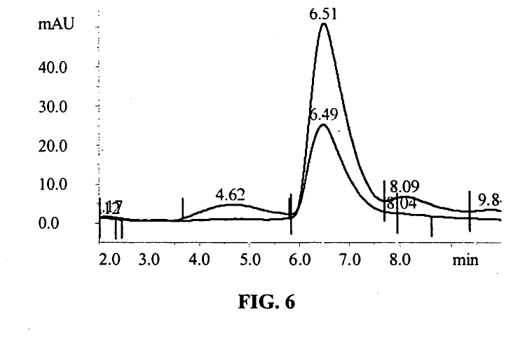

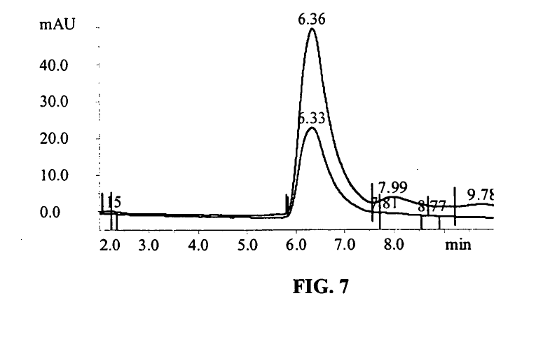

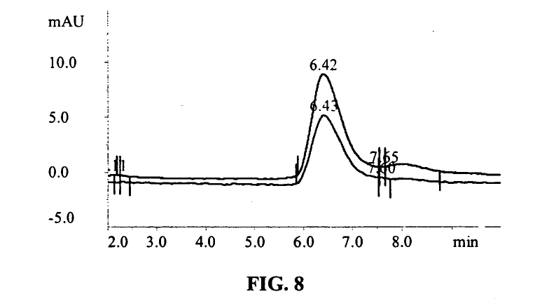

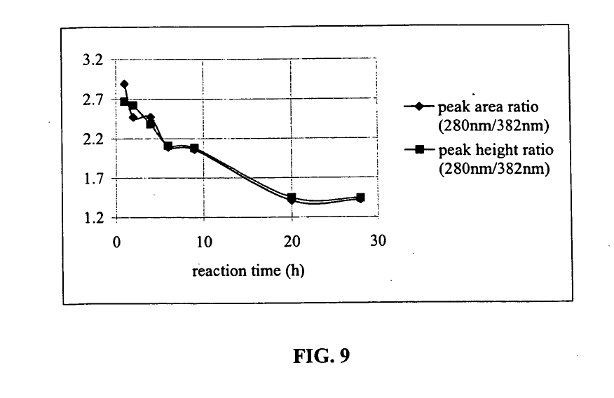

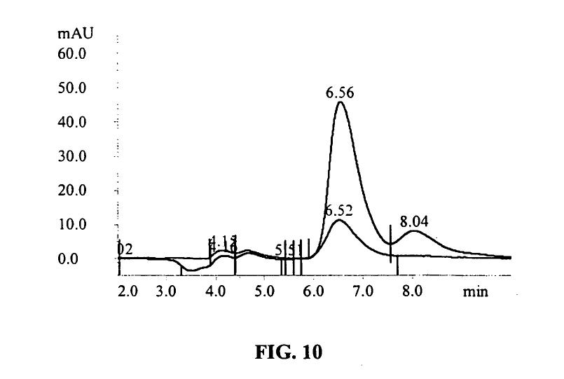

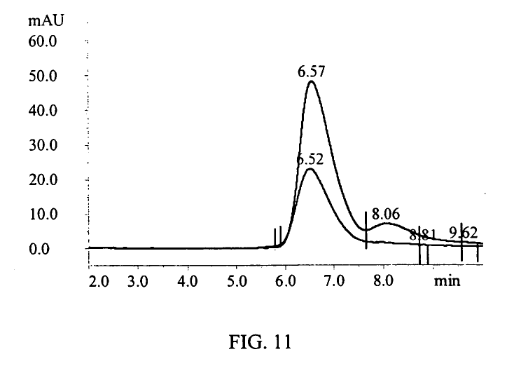

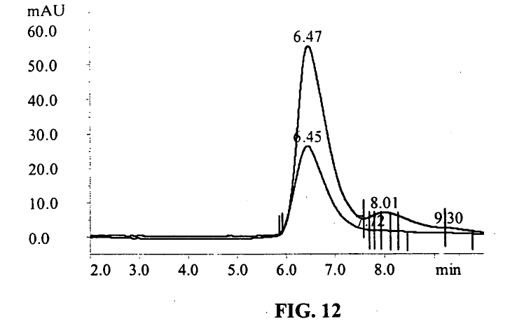

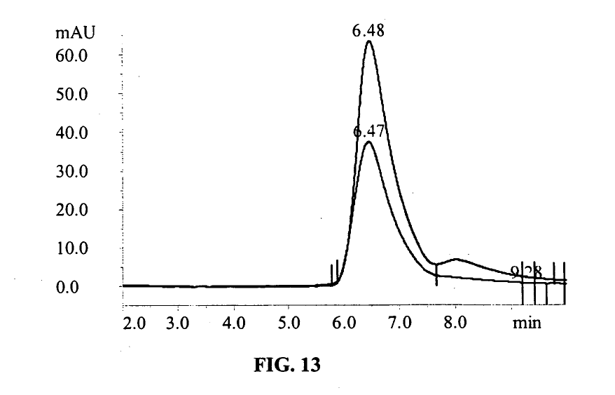

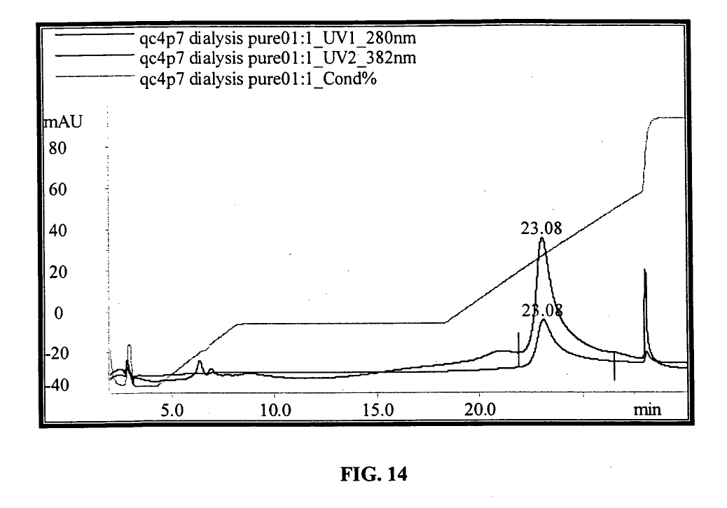

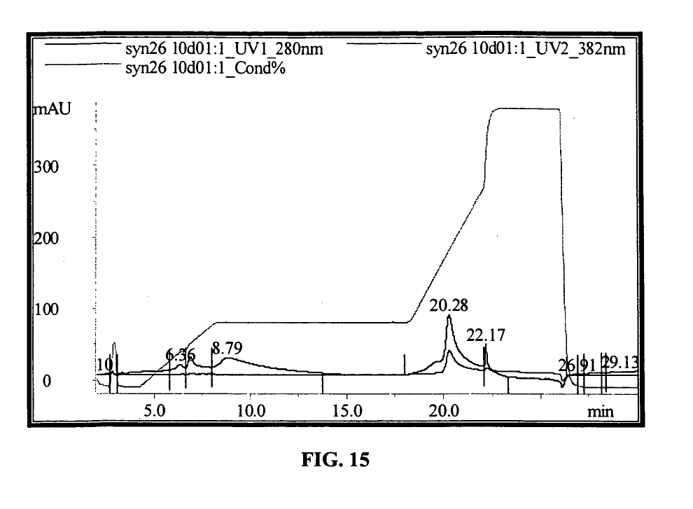

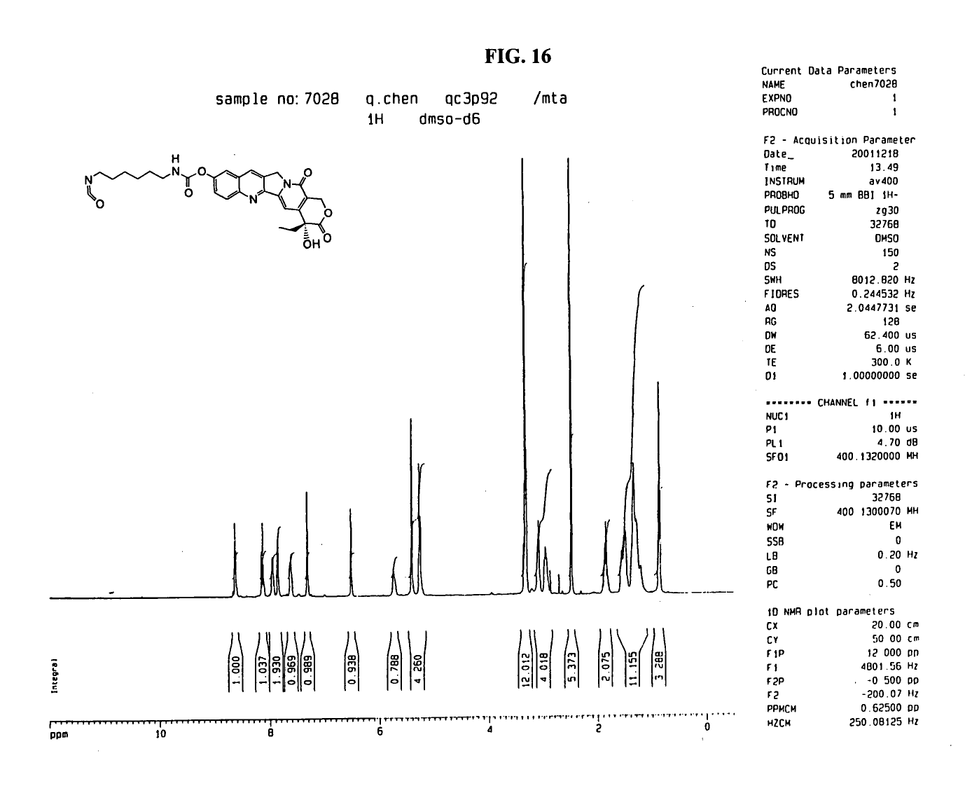

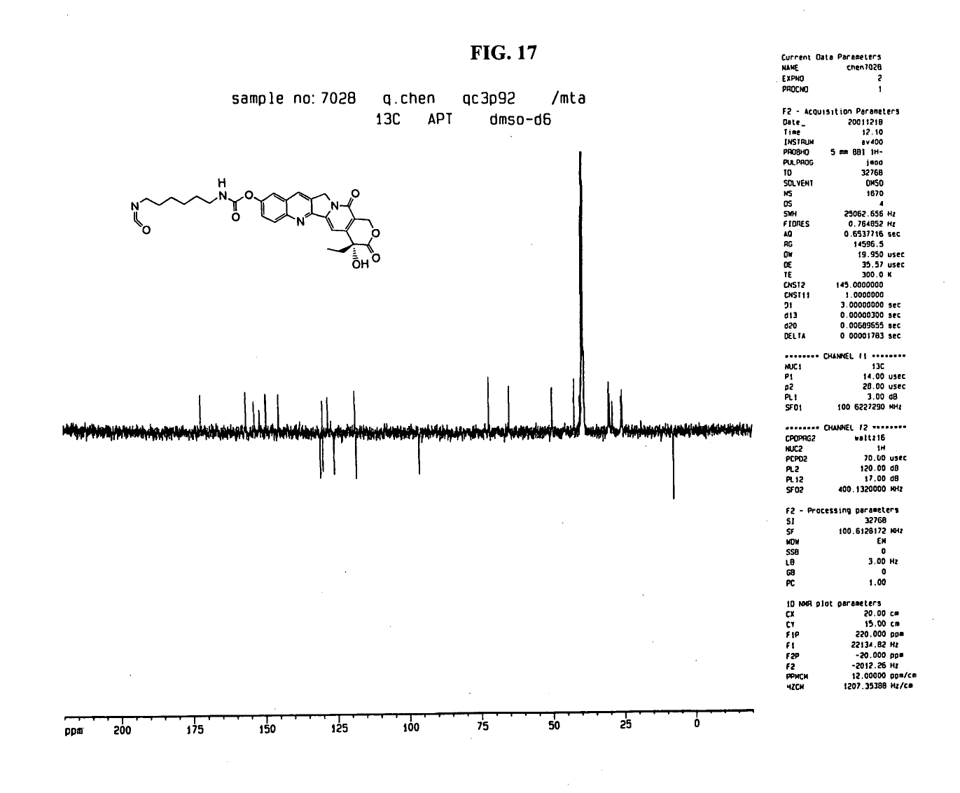

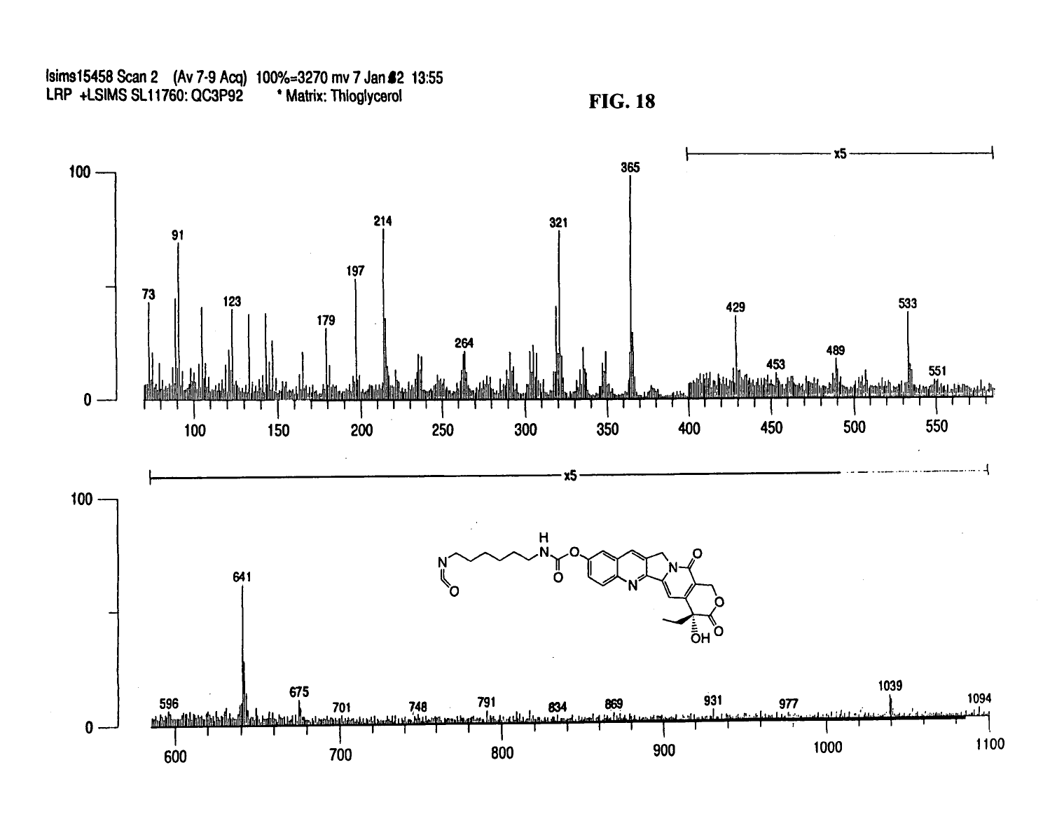

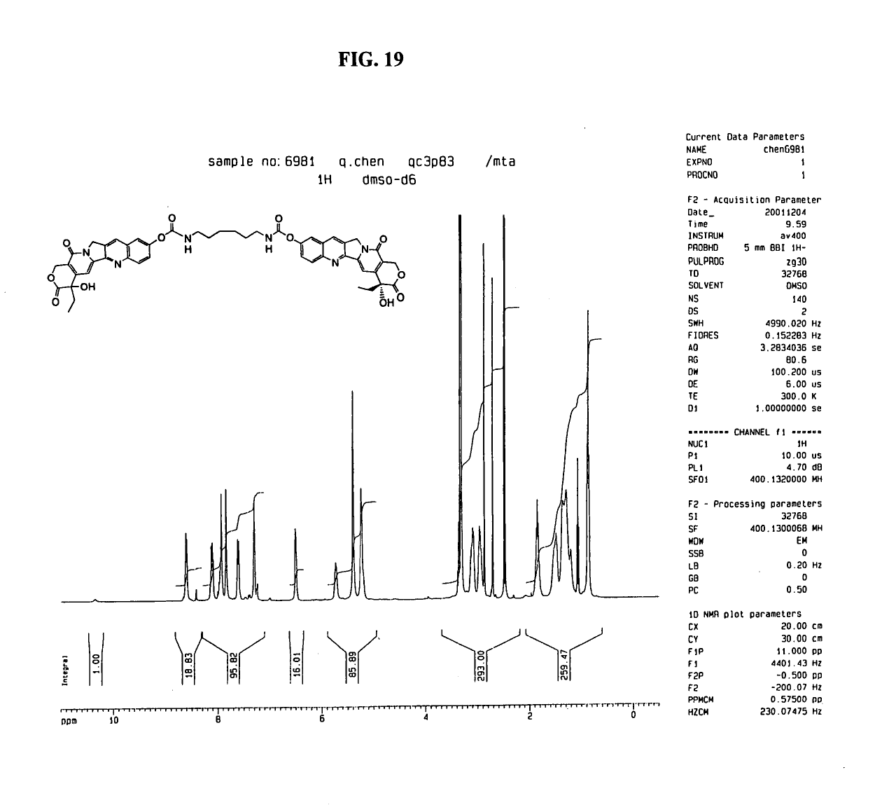

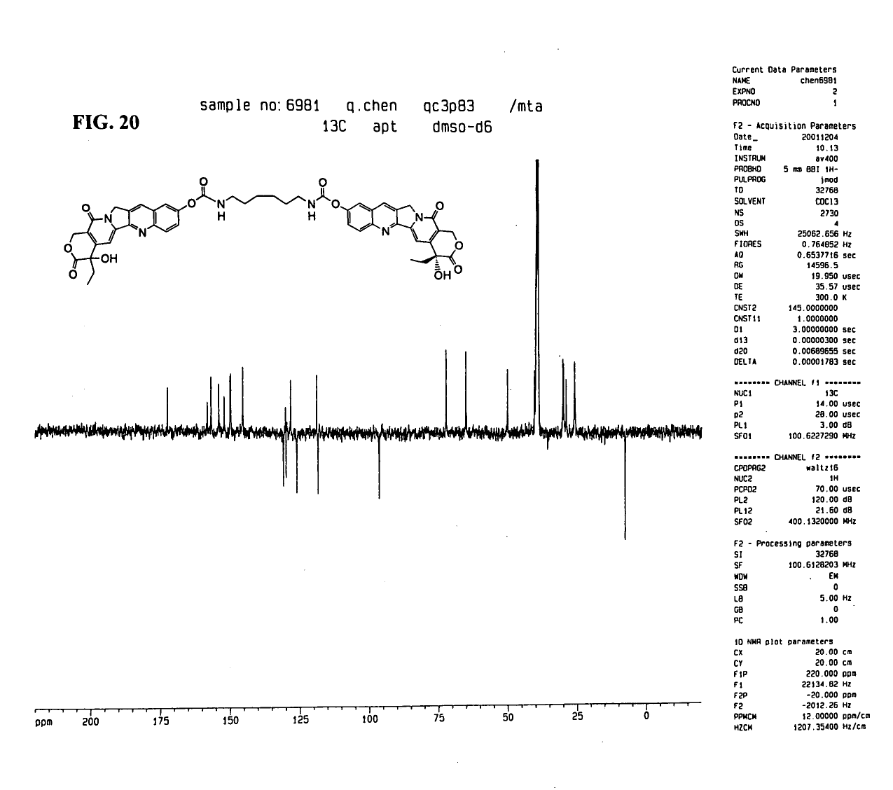

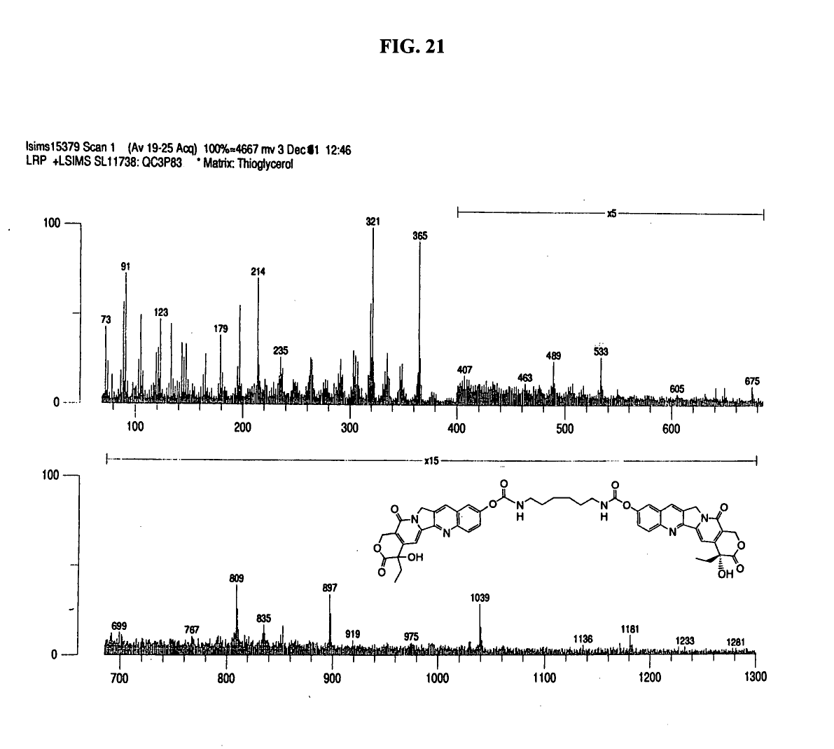

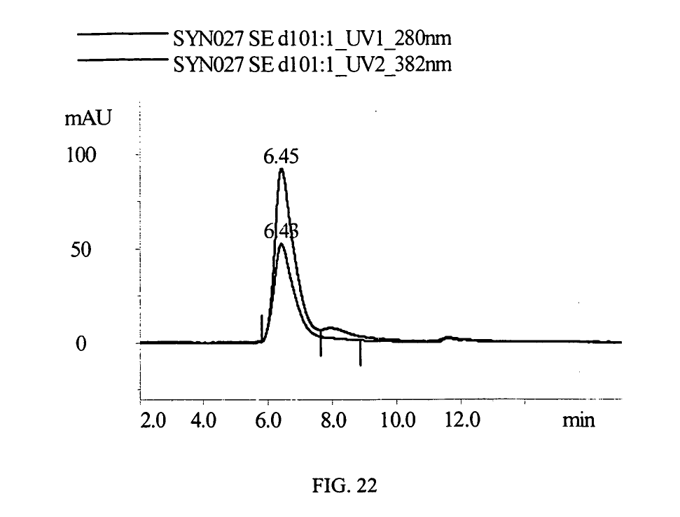

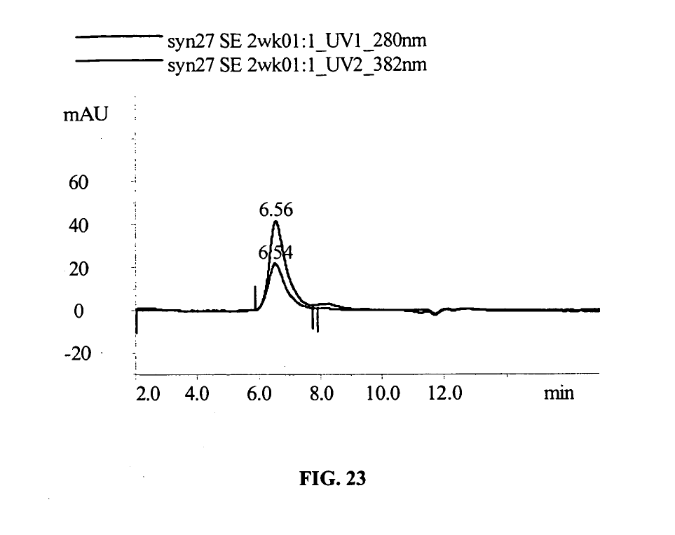

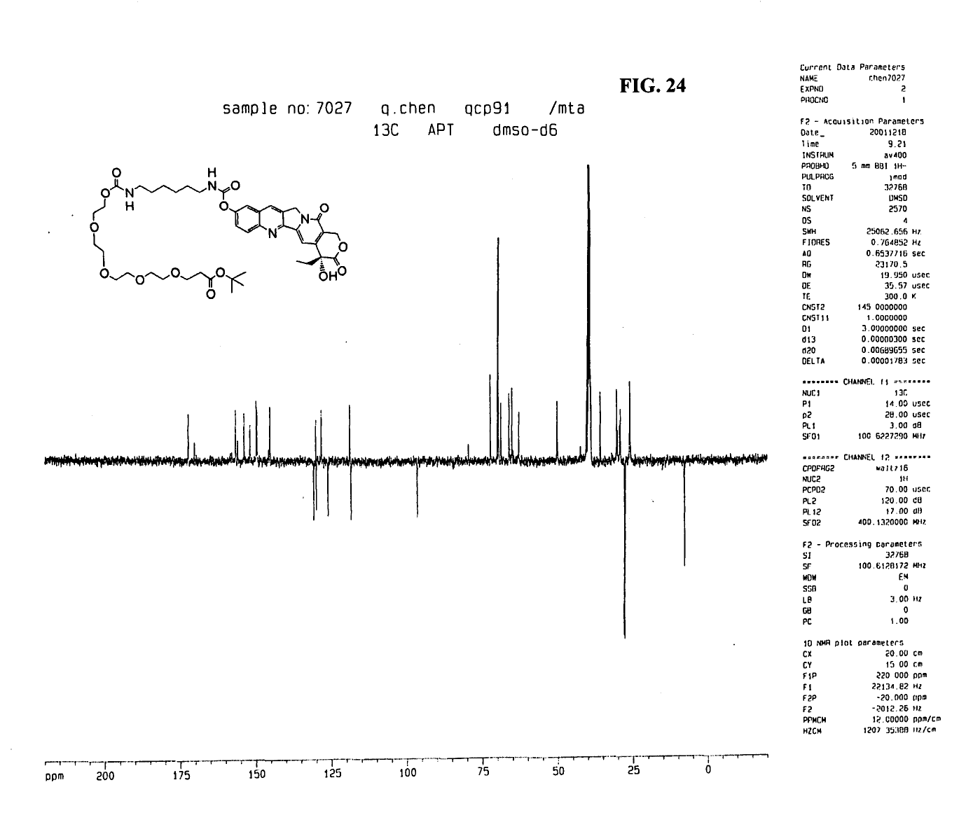

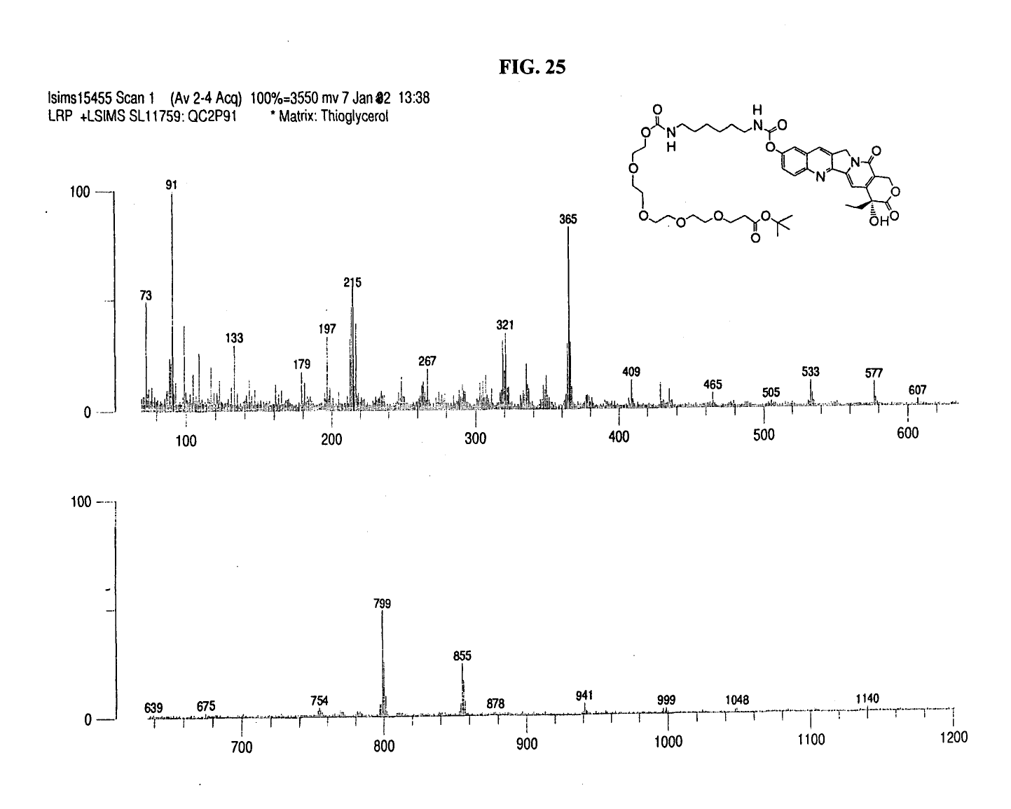

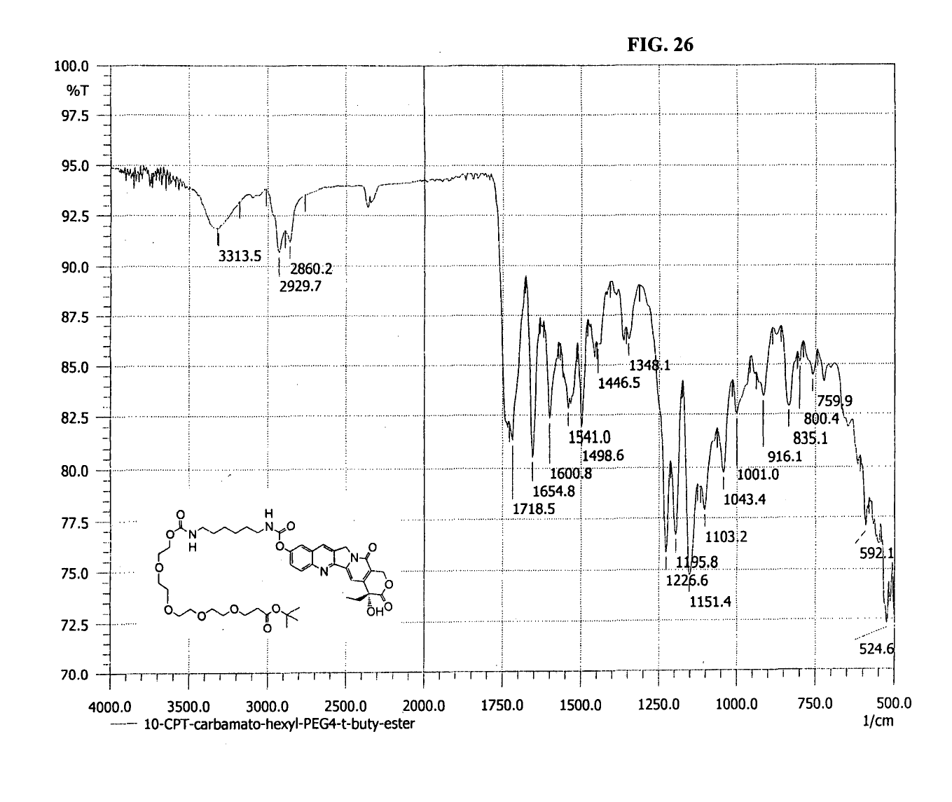

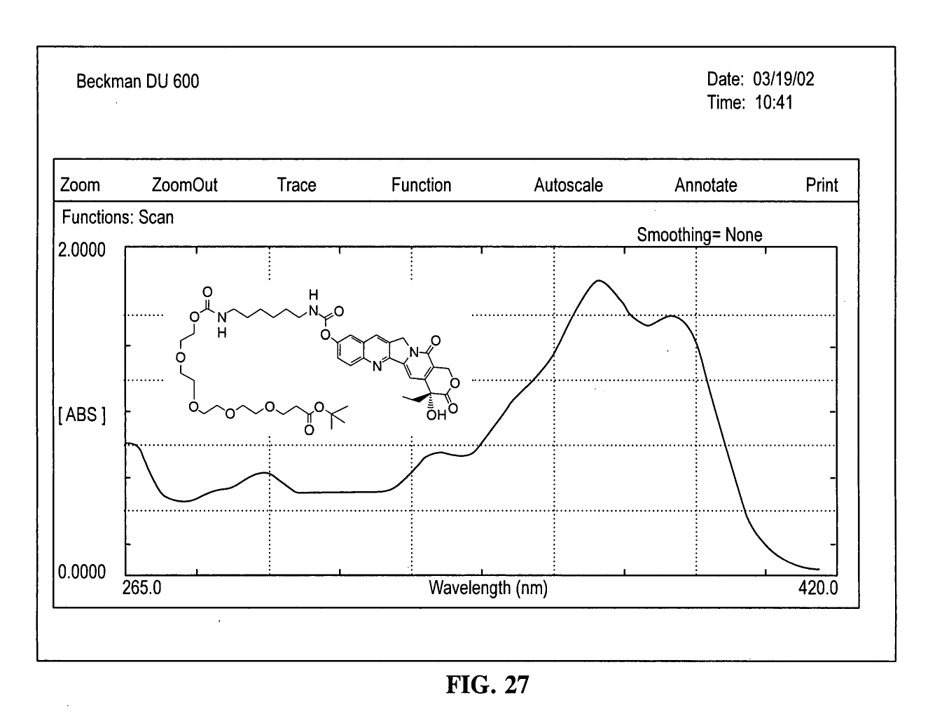

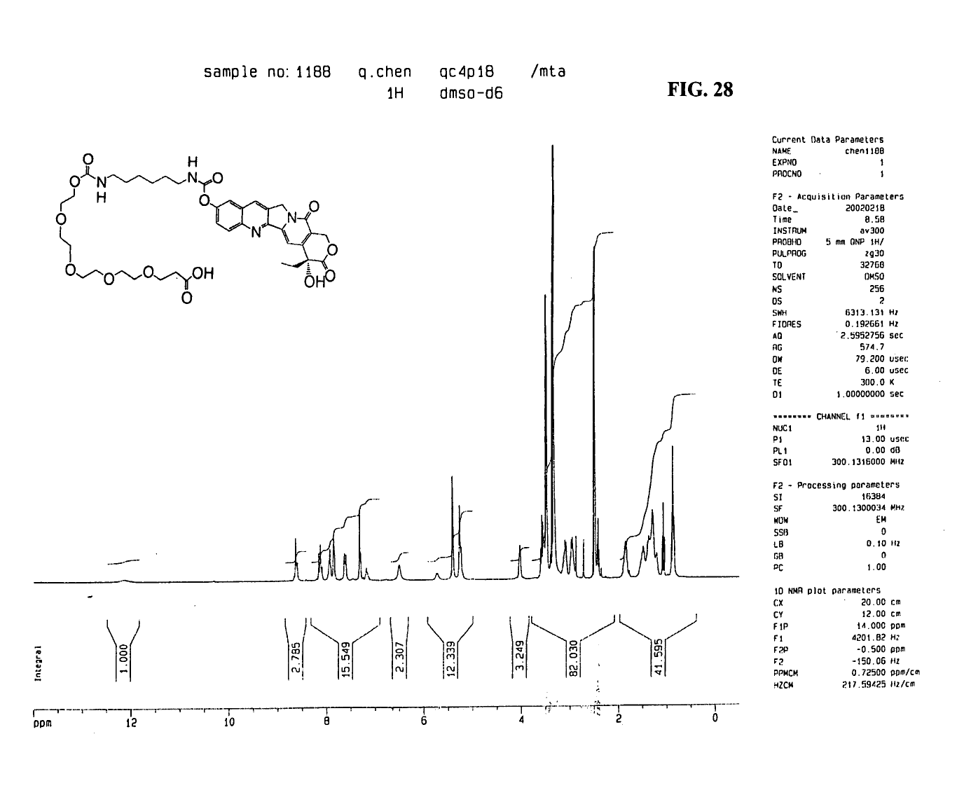

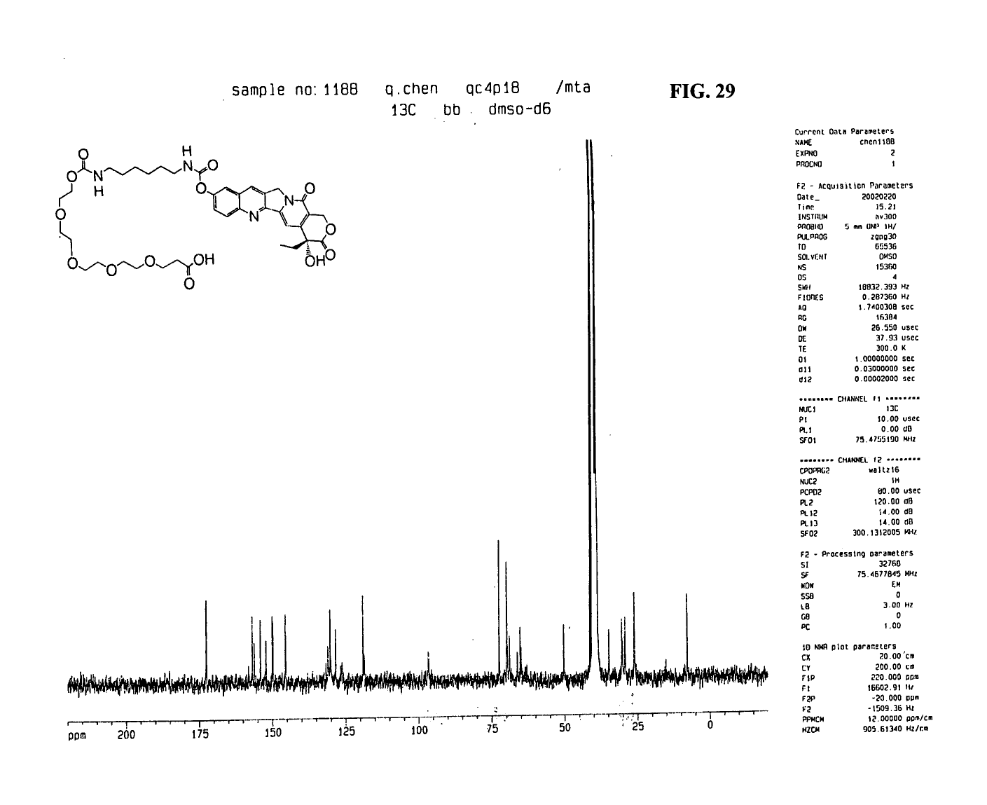

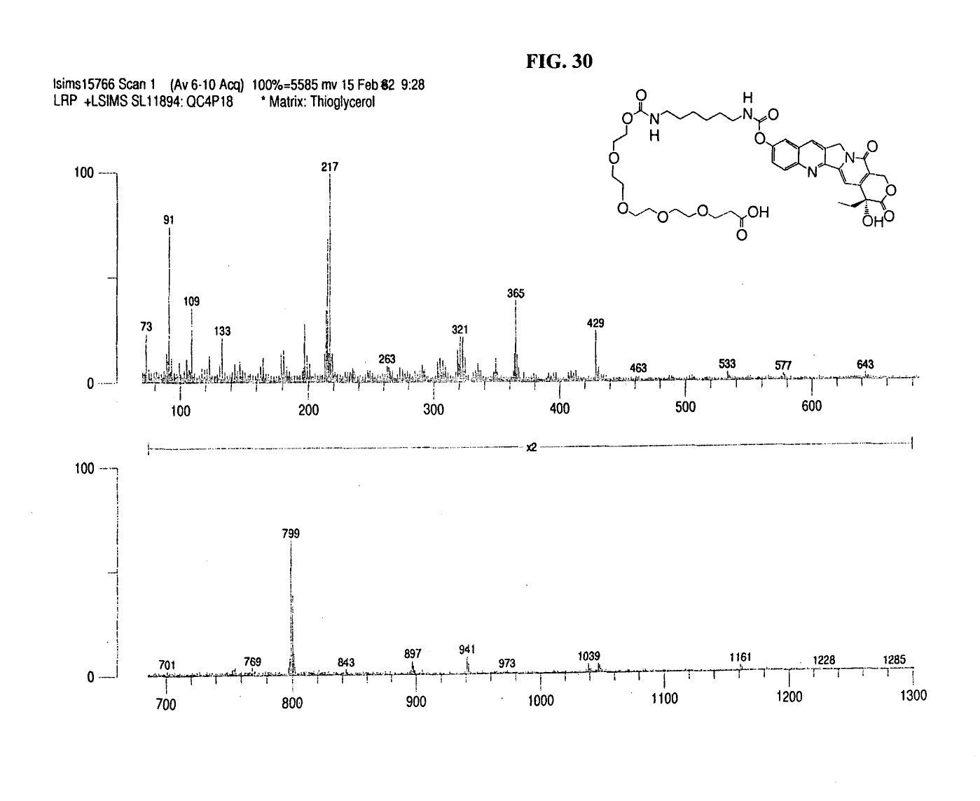

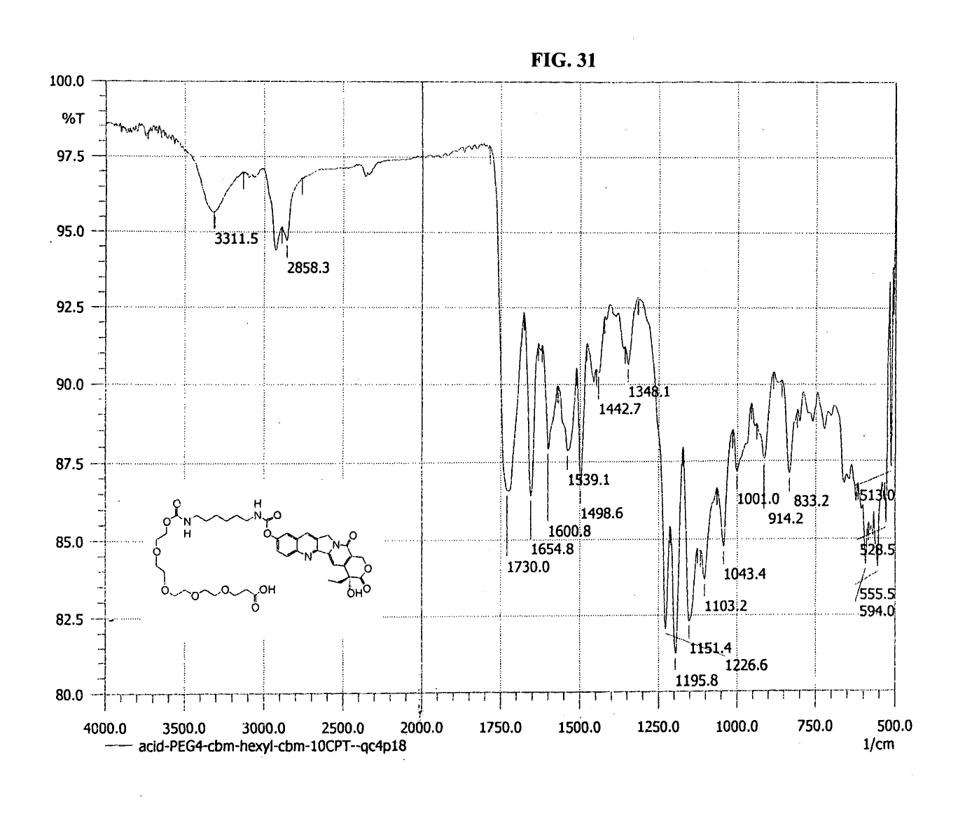

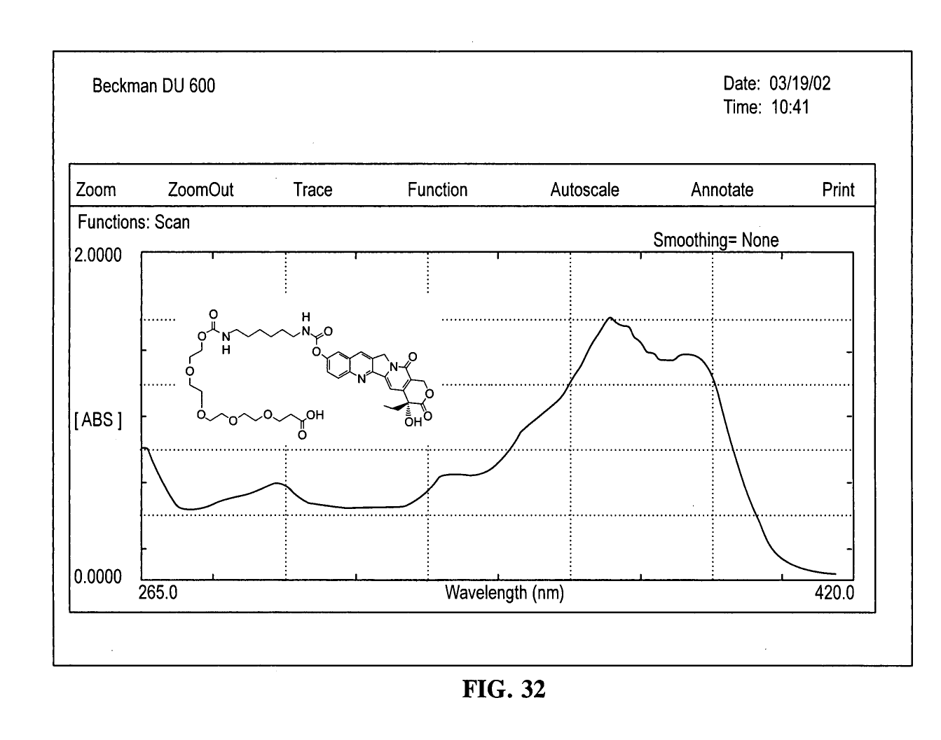

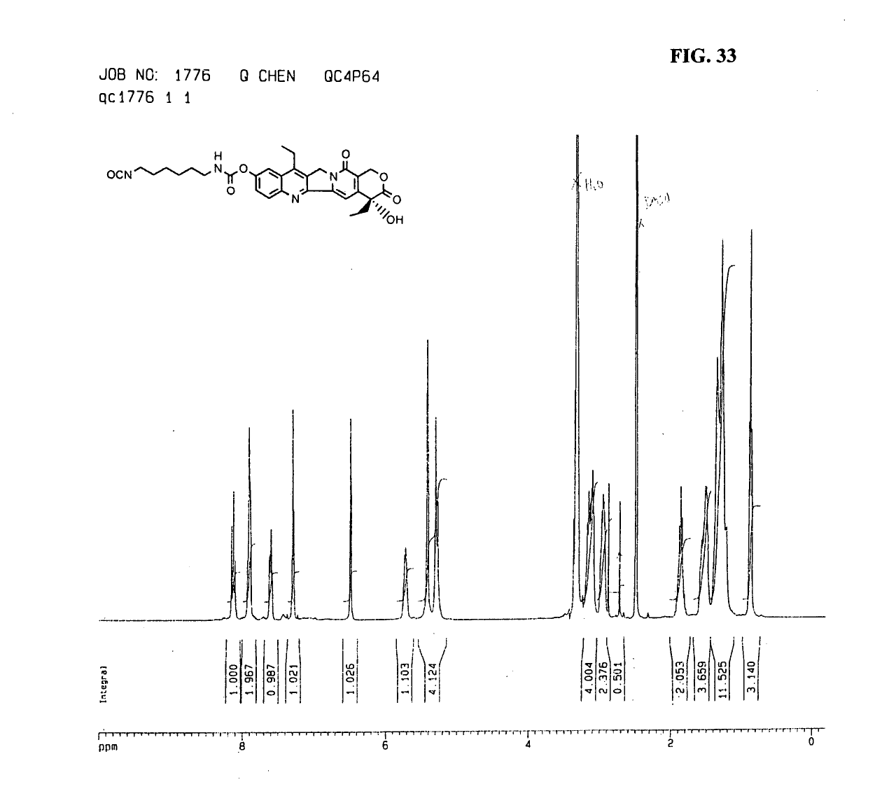

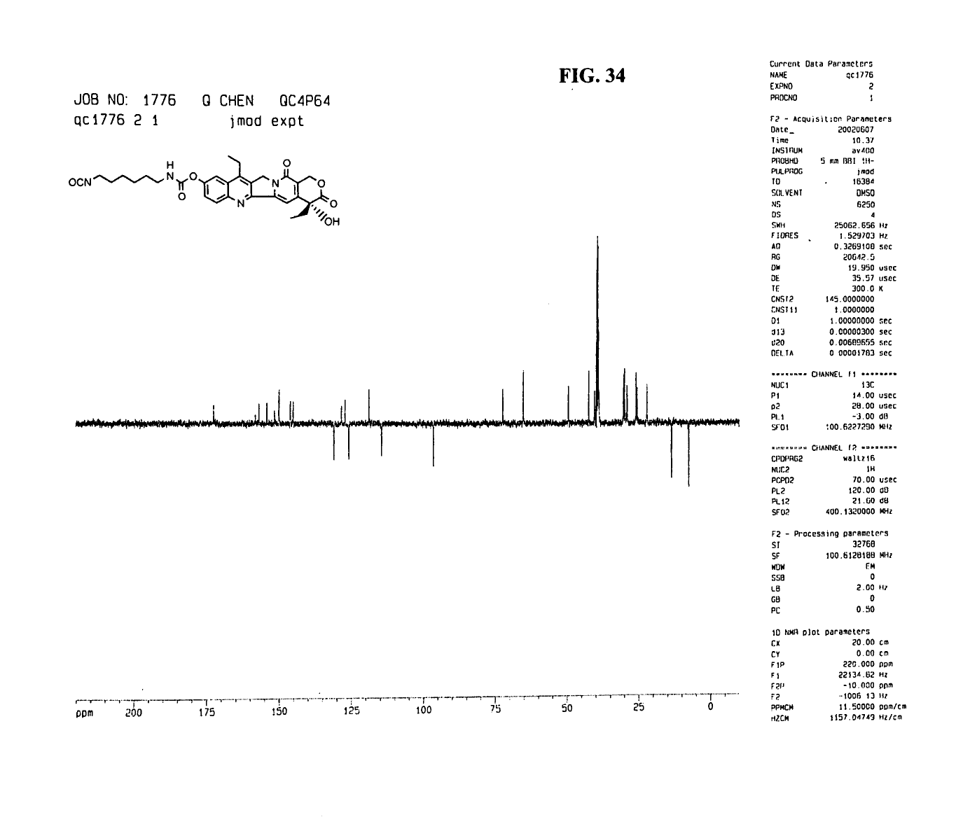

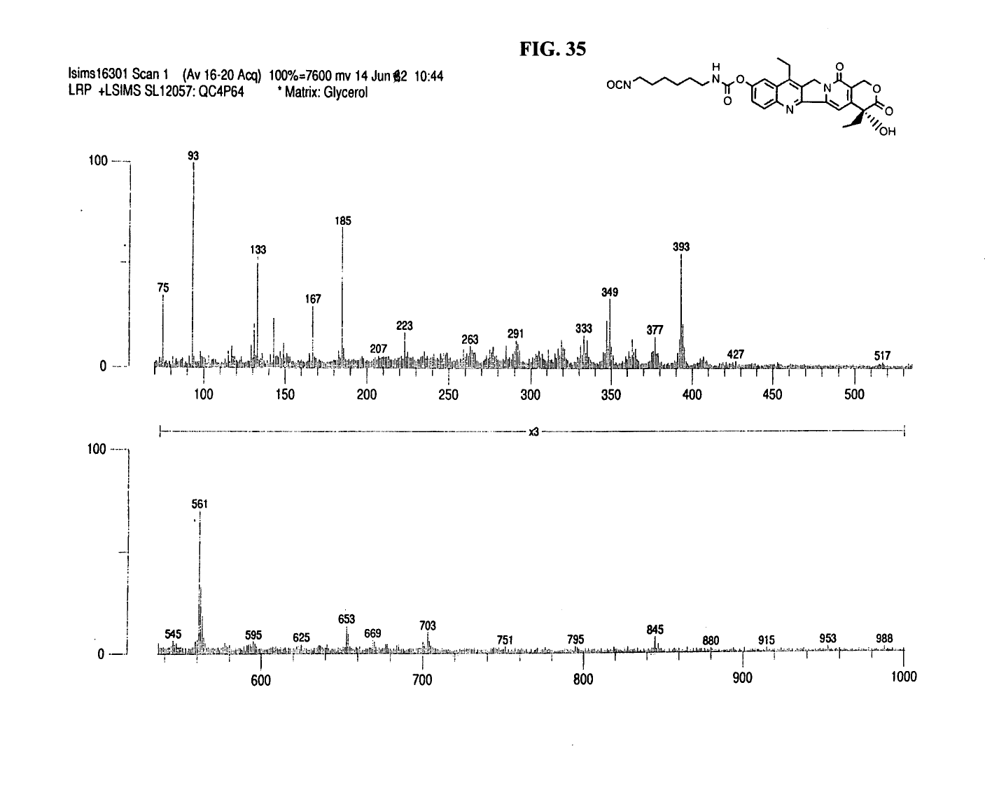

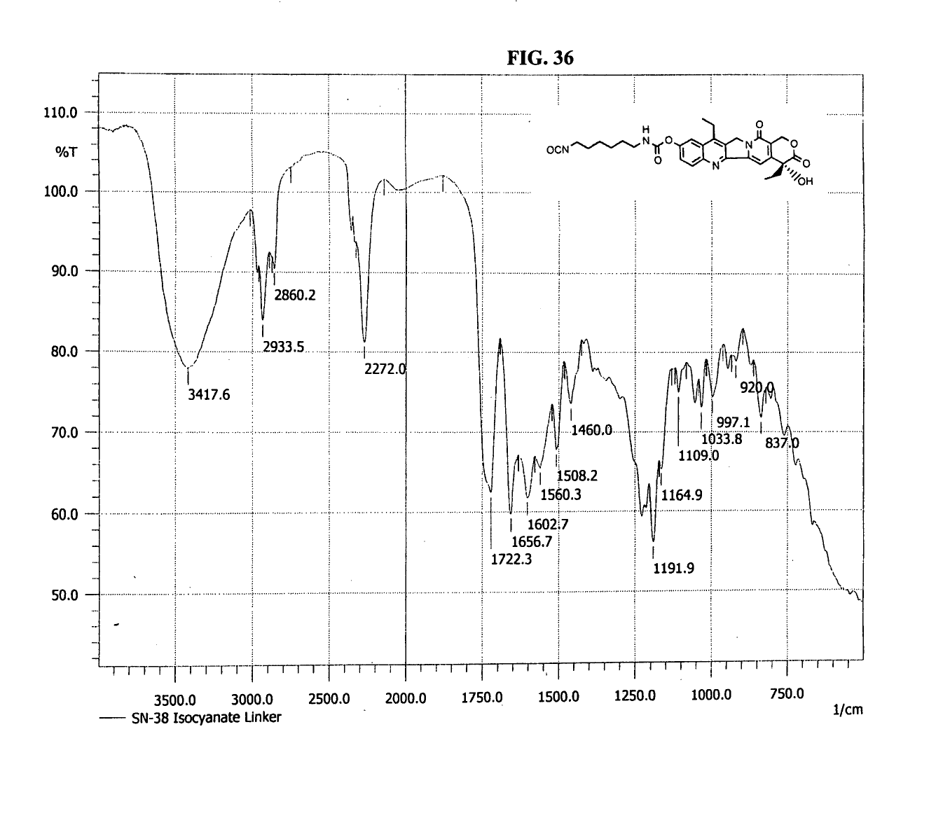

Reaction conditions are the following: DMF=30%, reaction time 3 hours at room temperature, p97 concentration 1.23 mg/mL, pH=7.40. Column: BioSep 300 size exclusive, eluent buffer: 0.1 M sodium phosphate monobasic, pH 6.8, flow rate: 1.0 ml/mL. Running method is the same as used in FIGS. 3-8. FIG. 10 shows the effect of 30 molar equivalents excess to the MSR. FIG. 11 shows the effect of 50 molar equivalents excess to the MSR. FIG. 12 shows the effect of 75 molar equivalents excess to the MSR. FIG. 13 shows the effect of 100 molar equivalents excess to the MSR.

FIG. 14 shows FPLC of SYN026 for the first day. Mono Q HR 10/10 ion-exchange column, buffer A: Tris (20 mM, pH 7.5), Buffer B Tris (20 mM, pH 7.5) containing 1M NaCl. The column parameters are depicted in the following table:

Variables

| Column | Mono_Q_HR_10/10 | |
|---|---|---|
| Wavelength_1 | 280 | {nm} |
| Wavelength_2 | 382 | {nm} |
| Wavelength_3 | 350 | {nm} |
| UV_Averaging_time | 5.12 | {sec} |
| Pressure_limit | 5 | {MPa} |
| Eluent_A_inlet | A1 | |
| Eluent_B_inlet | B1 | |
| Start_ConcB | 0.00 | {% B} |
| Flow_rate | 4.00 | {ml/min} |
| Equilibrate_with | .5 | {CV} |
| Flowthrough_FracSize | 0.000 | {ml} |
| Empty_loop_with | 1.2 | {ml} |
| Wash_column_with | .5 | {CV} |
| Start_Frac_at | 0 | {% B} |
| Eluate_FracSize | 0.000 | {ml} |
| End_Frac_at | 0 | {% B} |
| Target_ConcB_1 | 20 | {% B} |
| Length_of_gradient_1 | 2 | {base} |
| Target_ConcB_2 | 20 | {% B} |
| Length_of_gradient_2 | 5 | {base} |
| Target_ConcB_3 | 70 | {% B} |
| Length_of_gradient_3 | 5 | {base} |
| Conc_of_eluent_B | 100 | {% B} |
| Clean_with | 2 | {CV} |
| Reequilibrate_with | 2 | {CV} |

FIG. 15 shows FPLC of SYN026 after 10 days. Mono Q HR 10/10 ion-exchange column, buffer A: Tris (20 mM, pH 7.5), Buffer B Tris (20 mM, pH 7.5) containing 1M NaCl. The column parameters are depicted in the following table:

Variables

| Column | Mono_Q_HR_10/10 | |
|---|---|---|
| Wavelength_1 | 280 | {nm} |
| Wavelength_2 | 382 | {nm} |
| Wavelength_3 | 350 | {nm} |
| UV_Averaging_time | 5.12 | {sec} |
| Pressure_limit | 5 | {MPa} |
| Eluent_A_inlet | A1 | |
| Eluent_B_inlet | B1 | |
| Start_ConcB | 0.00 | {% B} |
| Flow_rate | 4.00 | {ml/min} |
| Equilibrate_with | .5 | {CV} |
| Flowthrough_FracSize | 0.000 | {ml} |
| Empty_loop_with | 1.2 | {ml} |
| Wash_column_with | .5 | {CV} |
| Start_Frac_at | 0 | {% B} |
| Eluate_FracSize | 0.000 | {ml} |
| End_Frac_at | 0 | {% B} |
| Target_ConcB_1 | 20 | {% B} |
| Length_of_gradient_1 | 2 | {base} |
| Target_ConcB_2 | 20 | {% B} |
| Length_of_gradient_2 | 5 | {base} |
| Target_ConcB_3 | 70 | {% B} |
| Length_of_gradient_3 | 2 | {base} |
| Conc_of_eluent_B | 100 | {% B} |
| Clean_with | 2 | {CV} |
| Reequilibrate_with | 5 | {CV} |

FIG. 16 shows ¹H NMR data, structure, and instrument parameters for NMR analysis of sample 7028 (mono isocyanate modified 10-hydroxycamptothecin).

FIG. 17 shows ¹³C NMR data, structure, and instrument parameters for NMR analysis of sample 7028.

FIG. 18 shows MS data for the structure analyzed in FIGS. 16 and 17.

FIG. 19 shows ¹H NMR data, structure, and instrument parameters for NMR analysis of sample 6981 (1,6-(bis(10-hydroxycamptothecincarbamate)hexane).

FIG. 20 shows ¹³C NMR data, structure, and instrument parameters for NMR analysis of sample 6981.

FIG. 21 shows MS data for the structure analyzed in FIGS. 19 and 20.

FIG. 22 shows an exemplary FPLC trace of SYN027 at 280 nm and at 382 nm detection for the first day. BioSep size exclusive column washing buffer: 0.1 M sodium phosphate, monobasic, pH=6.80. The column parameters for this FPLC run were as depicted in the following table:

Variables

| Column | 7 | {ml} |
|---|---|---|
| Wavelength_1 | 280 | {nm} |
| Wavelength_2 | 382 | {nm} |
| Wavelength_3 | 420 | {nm} |
| Pressure_Limit | 5 | {MPa} |
| Averaging_Time_UV | 5.12 | {sec} |
| Flow_rate | 1.00 | {ml/min} |
| Equilibrate_with | 0.5 | {CV} |
| Empty_loop_with | 0.5 | {ml} |
| Eluate_Frac_Size | 0.000 | {ml} |
| Start_Eluate_Frac_at | NextTube | |
| Peak_Frac_Size | 0.000 | {ml} |
| Start_Peak_Frac_at | NextTube | |
| Peak_Start_Slope | 100.00 | {mAU/min} |
| Peak_End_Slope | 75.00 | {mAU/min} |
| Minimum_Peak_Width | 0.15 | {min} |
| Length_of_elution | 5 | {CV} |

Scouting
Questions

| | |
|---|---|
| No 1: | Sample volume and type: |
| No 2: | Column: |
| No 3: | Eluent: |

FIG. 23 shows an exemplary FPLC trace of SYN027 at 280 nm and at 382 nm detection two weeks after its synthesis. BioSep size exclusive column washing buffer: 0.1 M sodium phosphate, monobasic, pH 6.80. The column parameter for this FPLC run were as depicted in the following table:

Variables

| Column | 7 | {ml} |
|---|---|---|
| Wavelength_1 | 280 | {nm} |
| Wavelength_2 | 382 | {nm} |
| Wavelength_3 | 420 | {nm} |
| Pressure_Limit | 5 | {MPa} |
| Averaging_Time_UV | 5.12 | {sec} |
| Flow_rate | 1.00 | {ml/min} |
| Equilibrate_with | 0.5 | {CV} |
| Empty_loop_with | 0.5 | {ml} |
| Eluate_Frac_Size | 0.000 | {ml} |
| Start_Eluate_Frac_at | NextTube | |
| Peak_Frac_Size | 0.000 | {ml} |
| Start_Peak_Frac_at | NextTube | |
| Peak_Start_Slope | 100.00 | {mAU/min} |
| Peak_End_Slope | 75.00 | {mAU/min} |
| Minimum_Peak_Width | 0.15 | {min} |
| Length_of_elution | 5 | {CV} |

Scouting
Questions

| | |
|---|---|
| No 1: | Sample volume and type: |
| No 2: | Column: |
| No 3: | Eluent: |

FIG. 24 shows ¹³C NMR data, structure, and instrument parameters for NMR analysis of sample 7027 (tert-Butyl-PEG4-carbamato-hexyl-carbamato-10-hydroxycamptothecin).

FIG. 25 shows MS data for the structure analyzed in FIG. 24.

FIG. 26 shows an IR spectrum for the structure analyzed in FIG. 24.

FIG. 27 shows the UV spectrum for the structure analyzed in FIG. 24.

FIG. 28 shows ¹H NMR data, structure, and instrument parameters for NMR analysis of sample 1188 (acid-PEG4-cbm-hexyl-cbm-10CPT).

FIG. 29 shows ¹³C NMR data, structure, and instrument parameters for NMR analysis of sample 1188.

FIG. 30 shows MS data for the structure analyzed in FIGS. 28 and 29.

FIG. 31 shows an IR spectrum for the structure analyzed in FIGS. 28 and 29.

FIG. 32 shows the UV spectrum for the structure analyzed in FIGS. 28 and 29.

FIG. 33 shows ¹H NMR data, structure, and instrument parameters for NMR analysis of sample 1776 (reaction product of reaction of SN-38 with 1,6-diisocyanatohexane).

FIG. 34 shows ¹³C NMR data, structure, and instrument parameters for NMR analysis of sample 1776.

FIG. 35 shows MS data for the structure analyzed in FIGS. 33 and 34.

FIG. 36 shows an IR spectrum for the structure analyzed in FIGS. 33 and 34.

### DETAILED DESCRIPTION OF THE INVENTION

Bioconjugates and preferred embodiments according to the present invention are of the formula 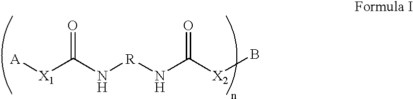
in which A is an active agent or drug comprising an active hydroxy or amino functionality; and B is a targeting or delivery biopolymer comprising an active hydroxy or amino functionality; and X₁ and X₂ are independently N or O; R is a substituted alkyl or unsubstituted alkyl or unsubstituted or substituted heteroalkyl from 1 to about 30 atoms in length or 1 to 50 atoms in length; and n is from 1 to 30. Where n is greater than 1, the active agents may be the same or different. Where different, the active agents are useful for the treatment of the same disease or condition. “Alkyl” encompasses divalent radicals of alkanes as defined below.

In a further embodiment, a label, L, is covalently attached to a compound of Formula I. The label may be attached to the bioconjugate at the active agent portion, the biopolymer portion, or the linker joining the active agent to the biopolymer: 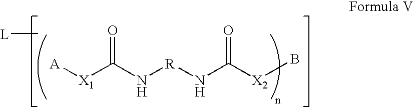
In some embodiments, the label is preferably attached to the biopolymer portion of a bioconjugate.

These bioconjugates have the advantage of release ability. When an isocyanate reagent according to the invention reacts with a hydroxy group it forms a carbamate bond, which can be hydrolyzed by endogenous enzymes (e.g., proteases) in the body of a subject to which it is administered. The isocyanate reagents according to the invention react with an amino group to generate an isourea bond, which can also be hydrolyzed by endogenous enzymes in the body of a subject to which it is administered. By virtue of attachment to the biopolymer, the drug conjugate is delivered to the target compartment or site in the body, the protease or other endogenous enzyme hydrolyzes the carbamate or isourea bond to release the free drug at the target site or compartment.

An exemplary bioconjugate comprises a biopolymer (e.g. a transcytosis protein such as p97) covalently linked through functional group, as is well known in the art of PEGylated peptides and proteins to a PEG moiety which is in turned linked via a carbamate linkage to the active agent or drug. In another embodiment, the conjugate is covalently linked through a carbamate group to a PEG moiety which is in turned linked via a carbamate linkage to the active agent or drug. In another embodiment, the conjugate is covalently linked through a carbamate group to a PEG moiety which is in turned linked via a carbamate linkage to an alkyl or homoalkyl moiety which is in turned linked via a carbamate linkage to the active agent or drug. In one embodiment, the active agent is 10-hydroxycamptothecin, the PEG moiety is any one of PEG3-PEG20, (e.g., PEG4, PEG5, PEG6), and the alkyl linkage is homoalkyl (e.g., butyl, pentyl, hexyl) and the biopolymer is soluble p97 or a portion thereof. For example, an exemplary p97 bioconjugate with 10-hydroxycamptothecin is of the formula:

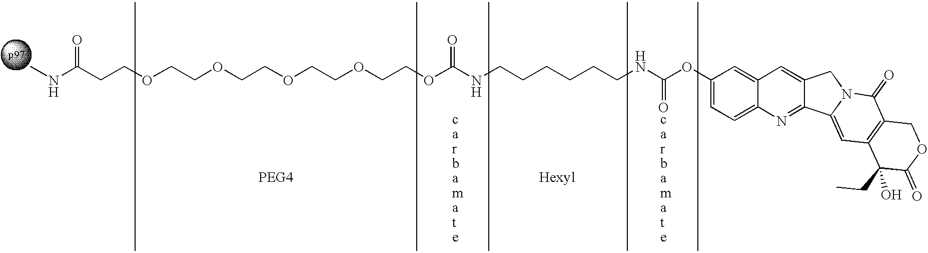

These bioconjugates also have the advantage of being synthesized with high efficiencies according to the inventive methods. The inventive reactions between isocyanate groups with hydroxy and amino are very efficient; and the yields are very high (usually over 90%). The synthesis of the modified small drug molecules with a biopolymer is also convenient and can be done in just one step. In addition, the new bond formed by the reaction of an isocyanate group with a hydroxy or an amino group will increase aqueous solubility of the drug. This property is important and very useful.

Definitions

Unless otherwise defined, all technical and scientific terms used herein have the same meaning as commonly understood by one of ordinary skill in the art to which this invention belongs. Each publication, patent application, patent, and other reference cited herein is incorporated by reference in its entirety to the extent that it is not inconsistent with the present disclosure.

It is noted here that, as used in this specification, the singular forms “a,” “an,” and “the” include plural reference unless the context clearly dictates otherwise.

Where substituent groups are specified by their conventional chemical formulae, written from left to right, they equally encompass the chemically identical substituents which would result from writing the structure from right to left, e.g., —CH₂O— is intended to also recite —OCH₂—.

Compounds of the Invention Generally

Compounds of the invention may contain one or more asymmetric centers and can thus occur as racemates and racemic mixtures, single enantiomers, diastereomeric mixtures and individual diastereomers. The present invention is meant to cover all such isomeric forms of the inventive compounds.

Such compounds of the invention may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid such as a resolving agent.

Alternatively, any enantiomer of such a compound of the invention may be obtained by stereospecific synthesis using optically pure starting materials of known configuration.

The bioconjugates and reagents of the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention.

The instant bioconjugates may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counter-ion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

The invention also encompasses prodrugs of the active agents which before or after hydrolysis of the bioconjugate undergo chemical conversion by metabolic processes before becoming active pharmacological substances. In general, such prodrugs will be derivatives of the bioconjugates that are readily convertible in vivo into a functional compound of the invention. Conventional procedures for the selection and preparation of suitable prodrug derivatives are described, for example, in “Design of Prodrugs”, ed. H. Bundgaard, Elsevier, 1985. The invention also encompasses active metabolites of active agents as active agents themselves.

Some of the compounds described herein contain olefinic double bonds, and unless specified otherwise, are meant to include both E and Z geometric isomers.

Some of the active agents and compounds described herein may exist with different points of attachment of hydrogen, referred to as tautomers. Such an example may be a ketone and its enol form known as keto-enol tautomers. The individual tautomers as well as mixture thereof are encompassed by the inventive formulas.

Alternatively, any enantiomer of an inventive bioconjugate or active agent or reagent or other compound of the invention may be obtained by stereospecific synthesis using optically pure starting materials or reagents of known configuration.

As used herein, the term “heteroatom” is meant to include oxygen (O), nitrogen (N), sulfur (S) and silicon (Si).

“Alkyl,” by itself or as part of another substituent, means, unless otherwise stated, a straight or branched chain, or cyclic hydrocarbon radical, or combination thereof, which may be fully saturated, mono- or polyunsaturated and can include the corresponding di- and multivalent radicals. In some embodiments, the alkyl portion has the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl” and “alkylene,” “cycloalkyl” and “heterocycloalkyl.” Alkyl groups which are limited to hydrocarbon groups are termed “homoalkyl.” Where an alkane or alkyl member is designated as R in Formula I, for instance, the corresponding divalent alkyl radical is indicated. For example, where R is designated as methane, methyl, or methylene, the corresponding compound of Formula I would be

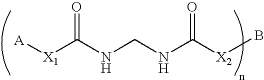

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. The term “heteroalkyl” encompasses “heteroalkylene.” The term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—; —CH₂—CH₂—O—CH₂—, —CH₂—CH₂NH—CH₂—, —CH₂—CH₂—N(CH₃)—CH₂—, —CH₂—S—CH₂—CH₂—, —CH₂—CH₂, —S(O)—CH₂—, —CH₂—CH₂—S(O)₂—CH₂—, —CH═CH—O—CH₂—, —Si(CH₃)₃, —CH₂—CH═N—OCH₂—, and —CH═CH—N(CH₃)—CH₂—. Up to two heteroatoms may be consecutive, such as, for example, —CH₂—NH—OCH₂— and —H₂—O—Si(CH₃)₂CH₂—. In some embodiments, no more than one heteroatom is consecutive in the alkyl backbone of the chain linking the drug and the biopolymer. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CH₂O— represents both —CH₂O— and —OCH₂—.

Where an alkane or alkyl or alkylene member is designated as R in Formula I, for instance, the corresponding divalent alkyl radical is indicated. For example, where R is designated as methane, methyl, or methylene; the corresponding compound of Formula I would be

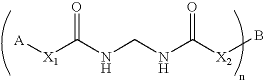

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexanol, 3-cyclohexanol, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The above terms “alkyl”, “heteroalkyl” and are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″—NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like)

Active Agents

Active agents according to the invention include agents that affect any biological process. The term “drug” or “therapeutic agent” refers to an active agent that has a pharmacological activity or benefits health when administered in a therapeutically effective amount. Examples of drugs or therapeutic agents include substances that are used in the prevention, diagnosis, alleviation, treatment or cure of a disease or condition.

The drug moiety as A may be any molecule, as well as any binding portion or fragment thereof, that is capable of modulating a biological process in a living host. Generally, A may be of any size, but is preferably a small organic molecule that is capable of binding to the target of interest. A drug moiety of the bioconjugate, when a small molecule, generally has a molecular weight of at least about 50 D, usually at least about 100 D, where the molecular weight may be as high as 500 D or higher, but will usually not exceed about 2000 D.

The drug moiety is capable of interacting with a target in the host into which the bioconjugate is administered during practice of the subject methods. The target may be a number of different types of naturally occurring structures, where targets of interest include both intracellular and extracellular targets, where such targets may be proteins, phospholipids, nucleic acids and the like, where proteins are of particular interest. Specific proteinaceous targets of interest include, without limitation, enzymes, e.g., kinases, phosphatases, reductases, cyclooxygenases, proteases and the like, targets comprising domains involved in protein-protein interactions, such as the SH2, SH3, PTB and PDZ domains, structural proteins, e.g., actin, tubulin, etc., membrane receptors, immunoglobulins, e.g., IgE, cell adhesion receptors, such as integrins, etc, ion channels, transmembrane pumps, transcription factors, signaling proteins, and the like.

The active agent or drug has a hydroxy or an amino group for reacting with the isocyanate reagent or the active agent is chemically modified to introduce a hydroxy or an amino group for reacting with the isocyanate reagent.

In some embodiments, the active agent or drug will also comprise a region that may be modified and/or participate in covalent linkage, preferably, without loss of the desired biological activity of the active agent. The drug moieties often comprise cyclical carbon or heterocyclic structures and/or aromatic or polyaromatic structures substituted with one or more of the above functional groups. Also of interest as drug moieties are structures found among biomolecules, proteins, enzymes, polysaccharides, and polynucleic acids, including peptides, saccharides, fatty acids, steroids, purines, pyrimidines, derivatives, structural analogs or combinations thereof.

The bioconjugate can comprise one or more active agents linked to the same biopolymer. For example, conjugation reactions may conjugate from 1 to 5, about 5, about 1-10, about 5 to 10, about 10-20, about 20-30, or 30 or more molecules of an active agent to the biopolymer. These formulations can be employed as mixtures, or they may be purified into specific (mol:mol) formulations. Those skilled in the art are able to determine which format and which mol:mol ratio is preferred. Further, more than one type of active agent may be linked to the biopolymer where delivery of more than one type of an agent to a target site or compartment is desired. A plurality of active agent species may be attached to the same biopolymers such as adriamycin-cisplatinum bioconjugate compositions where the biopolymer is a p97 related protein. Thus, the bioconjugates may consist of a range of mol:mol ratios and incorporate more than one type of active agent. These, too, may be separated into purified mixtures or they may be employed in aggregate. Active agents include those identified U.S. Pat. No. 6,372,712 which is incorporated herein by reference.

Specific drugs of interest from which the drug moiety may be derived include, but are not limited to: psychopharmacological agents, such as (1) central nervous system depressants, e.g., general anesthetics (barbiturates, benzodiazepines, steroids, cyclohexanone derivatives, and miscellaneous agents), sedative-hypnotics (benzodiazepines, barbiturates, piperidinediones and triones, quinazoline derivatives, carbamates, aldehydes and derivatives, amides, acyclic ureides, benzazepines and related drugs, phenothiazines, etc.), central voluntary muscle tone modifying drugs (anticonvulsants, such as hydantoins, barbiturates, oxazolidinediones, succinimides, acylureides, glutarimides, benzodiazepines, secondary and tertiary alcohols, dibenzazepine derivatives, valproic acid and derivatives, GABA analogs, etc.), analgesics (morphine and derivatives, oripavine derivatives, morphinan derivatives, phenylpiperidines, 2,6-methane-3-benzazocaine derivatives, diphenylpropylamines and isosteres, salicylates, p-aminophenol derivatives, 5-pyrazolone derivatives, arylacetic acid derivatives, fenamates and isosteres, etc.) and antiemetics (anticholinergics, antihistamines, antidopaminergics, etc.), (2) central nervous system stimulants, e.g., analeptics (respiratory stimulants, convulsant stimulants, psychomotor stimulants), narcotic antagonists (morphine derivatives, oripavine derivatives, 2,6-methane-3-benzoxacine derivatives, morphinan derivatives) nootropics, (3) psychopharmacologicals, e.g., anxiolytic sedatives (benzodiazepines, propanediol carbamates) antipsychotics (phenothiazine derivatives, thioxanthine derivatives, other tricyclic compounds, butyrophenone derivatives and isosteres, diphenylbutylamine derivatives, substituted benzamides, arylpiperazine derivatives, indole derivatives, etc.), antidepressants (tricyclic compounds, MAO inhibitors, etc.), (4) respiratory tract drugs, e.g., central antitussives (opium alkaloids and their derivatives); pharmacodynamic agents, such as (1) peripheral nervous system drugs, e.g., local anesthetics (ester derivatives, amide derivatives), (2) drugs acting at synaptic or neuroeffector junctional sites, e.g., cholinergic agents, cholinergic blocking agents, neuromuscular blocking agents, adrenergic agents, antiadrenergic agents, (3) smooth muscle active drugs, e.g., spasmolytics (anticholinergics, musculotropic spasmolytics), vasodilators, smooth muscle stimulants, (4) histamines and antihistamines, e.g., histamine and derivative thereof (betazole), antihistamines (H₁-antagonists, H₂-antagonists), histamine metabolism drugs, (5) cardiovascular drugs, e.g., cardiotonics (plant extracts, butenolides, pentadienolids, alkaloids from erythrophleum species, ionophores, adrenoceptor stimulants, etc), antiarrhythmic drugs, antihypertensive agents, antilipidemic agents (clofibric acid derivatives, nicotinic acid derivatives, hormones and analogs, antibiotics, salicylic acid and derivatives), antivaricose drugs, hemostyptics, (6) blood and hemopoietic system drugs, e.g., antianemia drugs, blood coagulation drugs (hemostatics, anticoagulants, antithrombotics, thrombolytics, blood proteins and their fractions), (7) gastrointestinal tract drugs, e.g., digestants (stomachics, choleretics), antiulcer drugs, antidiarrheal agents, (8) locally acting drugs; chemotherapeutic agents, such as (1) anti-infective agents, e.g., ectoparasiticides (chlorinated hydrocarbons, pyrethins, sulfurated compounds), anthelmintics, antiprotozoal agents, antimalarial agents, antiamebic agents, antileischmanial drugs, antitrichomonal agents, antitrypanosomal agents, sulfonamides, antimycobacterial drugs, antiviral chemotherapeutics, etc., and (2) cytostatics, i.e., antineoplastic agents or cytotoxic drugs, such as alkylating agents, e.g., Mechlorethamine hydrochloride (Nitrogen Mustard, Mustargen, HN2), Cyclophosphamide (Cytovan, Endoxana), Ifosfamide (IFEX), Chlorambucil (Leukeran), Melphalan (Phenylalanine Mustard, L-sarcolysin, Alkeran, L-PAM), Busulfan (Myleran), Thiotepa (Triethylenethiophosphoramide), Carmustine (BiCNU, BCNU), Lomustine (CeeNU, CCNU), Streptozocin (Zanosar) and the like; plant alkaloids, e.g., Vincristine (Oncovin), Vinblastine (Velban, Velbe), Paclitaxel (Taxol), and the like; antimetabolites, e.g., Methotrexate (MTX), Mercaptopurine (Purinethol, 6-MP), Thioguanine (6-TG), Fluorouracil (5-FU), Cytarabine (Cytosar-U, Ara-C), Azacitidine (Mylosar, 5-AZA) and the like; antibiotics, e.g., Dactinomycin (Actinomycin D, Cosmegen), Doxorubicin (Adriamycin), Daunorubicin (duanomycin, Cerubidine), Idarubicin (Idamycin), Bleomycin (Blenoxane), Picamycin (Mithramycin, Mithracin), Mitomycin (Mutamycin) and the like, and other anticellular proliferative agents, e.g., Hydroxyurea (Hydrea), Procarbazine (Mutalane), Dacarbazine (DTIC-Dome), Cisplatin (Platinol) Carboplatin (Paraplatin), Asparaginase (Elspar) Etoposide (VePesid, VP-16-213), Amsarcrine (AMSA, m-AMSA), Mitotane (Lysodren), Mitoxantrone (Novatrone), and the like;

Antibiotics, such as: aminoglycosides, e.g., amikacin, apramycin, arbekacin, bambermycins, butirosin, dibekacin, dihydrostreptomycin, fortimicin, gentamicin, isepamicin, kanamycin, micronomcin, neomycin, netilmicin, paromycin, ribostamycin, sisomicin, spectinomycin, streptomycin, tobramycin, trospectomycin; amphenicols, e.g., azidamfenicol, chloramphenicol, florfenicol, and theimaphenicol; ansamycins, e.g., rifamide, rifampin, rifamycin, rifapentine, rifaximin; .beta.-lactams, e.g., carbacephems, carbapenems, cephalosporins, cehpamycins, monobactams, oxaphems, penicillins; lincosamides, e.g., clinamycin, lincomycin; macrolides, e.g., clarithromycin, dirthromycin, erythromycin, etc.; polypeptides, e.g., amphomycin, bacitracin, capreomycin, etc.; tetracyclines, e.g., apicycline, chlortetracycline, clomocycline, etc.; synthetic antibacterial agents, such as 2,4-diaminopyrimidines, nitrofurans, quinolones and analogs thereof, sulfonamides, sulfones;

Antifungal agents, such as: polyenes, e.g., amphotericin B, candicidin, demmostatin, filipin, fungichromin, hachimycin, hamycin, lucensomycin, mepartricin, natamycin, nystatin, pecilocin, perimycin; synthetic antifungals, such as allylamines, e.g., butenafine, naftifine, terbinafine; imidazoles, e.g., bifonazole, butoconazole, chlordantoin, chlormidazole, etc., thiocarbamates, e.g., tolciclate, triazoles, e.g., fluconazole, itraconazole, terconazole;

Antihelmintics, such as: arecoline, aspidin, aspidinol, dichlorophene, embelin, kosin, napthalene, niclosamide, pelletierine, quinacrine, alantolactone, amocarzine, amoscanate, ascaridole, bephenium, bitoscanate, carbon tetrachloride, carvacrol, cyclobendazole, diethylcarbamazine, etc.;

Antimalarials, such as: acedapsone, amodiaquin, arteether, artemether, artemisinin, artesunate, atovaquone, bebeerine, berberine, chirata, chlorguamide, chloroquine, chlorprogaunil, cinchona, cinchonidine, cinchonine, cycloguanil, gentiopicrin, halofantrine, hydroxychloroquine, mefloquine hydrochloride, 3-methylarsacetin, pamaquine, plasmocid, primaquine, pyrimethamine, quinacrine, quinidine, quinine, quinocide, quinoline, dibasic sodium arsenate;

Antiprotozoan agents, such as: acranil, tinidazole, ipronidazole, ethylstibamine, pentamidine, acetarsone, aminitrozole, anisomycin, nifuratel, tinidazole, benzidazole, suramin, and the like.

Drug compounds of interest from which drug moieties may be derived are also listed in: Goodman & Gilman's, The Pharmacological Basis of Therapeutics (9th Ed) (Goodman et al. eds) (McGraw-Hill) (1996); and 1999 Physician's Desk Reference (1998).

Specific compounds of interest also include, but are not limited to:
antineoplastic agents, as disclosed in U.S. Pat. Nos. 5,880,161, 5,877,206, 5,786,344, 5,760,041, 5,753,668, 5,698,529, 5,684,004, 5,665,715, 5,654,484, 5,624,924, 5,618,813, 5,610,292, 5,597,831, 5,530,026, 5,525,633, 5,525,606, 5,512,678, 5,508,277, 5,463,181, 5,409,893, 5,358,952, 5,318,965, 5,223,503, 5,214,068, 5,196,424, 5,109,024, 5,106,996, 5,101,072, 5,077,404, 5,071,848, 5,066,493, 5,019,390, 4,996,229, 4,996,206, 4,970,318, 4,968,800, 4,962,114, 4,927,828, 4,892,887, 4,889,859, 4,886,790, 4,882,334, 4,882,333, 4,871,746, 4,863,955, 4,849,563, 4,845,216, 4,833,145,4,824,955, 4,785,085, 4,684,747, 4,618,685, 4,611,066, 4,550,187, 4,550,186, 4,544,501, 4,541,956, 4,532,327, 4,490,540, 4,399,283, 4,391,982, 4,383,994, 4,294,763, 4,283,394, 4,246,411, 4,214,089, 4,150,231, 4,147,798, 4,056,673, 4,029,661, 4,012,448;
psychopharmacological/psychotropic agents, as disclosed in U.S. Pat. Nos. 5,192,799, 5,036,070, 4,778,800,4,753,951, 4,590,180; 4,690,930, 4,645,773, 4,427,694, 4,424,202, 4,440,781, 5,686,482, 5,478,828, 5,461,062, 5,387,593, 5,387,586, 5,256,664, 5,192,799, 5,120,733, 5,036,070, 4,977,167, 4,904,663, 4,788,188, 4,778,800, 4,753,951, 4,690,930, 4,645,773, 4,631,285, 4,617,314, 4,613,600, 4,590,180, 4,560,684, 4,548,938, 4,529,727, 4,459,306, 4,443,451, 4,440,781, 4,427,694, 4,424,202, 4,397,853, 4,358,451, 4,324,787, 4,314,081, 4,313,896, 4,294,828, 4,277,476, 4,267,328, 4,264,499, 4,231,930, 4,194,009, 4,188,388, 4,148,796, 4,128,717, 4,062,858, 4,031,226, 4,020,072, 4,018,895, 4,018,779, 4,013,672, 3,994,898, 3,968,125, 3,939,152, 3,928,356, 3,880,834, 3,668,210;
cardiovascular agents, as disclosed in U.S. Pat. Nos. 4,966,967, 5,661,129, 5,552,411, 5,332,737, 5,389,675, 5,198,449, 5,079,247, 4,966,967, 4,874,760, 4,954,526, 5,051,423, 4,888,335, 4,853,391, 4,906,634, 4,775,757, 4,727,072, 4,542,160, 4,522,949, 4,524,151, 4,525,479, 4,474,804, 4,520,026, 4,520,026, 5,869,478, 5,859,239, 5,837,702, 5,807,889, 5,731,322, 5,726,171, 5,723,457, 5,705,523, 5,696,111, 5,691,332, 5,679,672, 5,661,129, 5,654,294, 5,646,276, 5,637,586, 5,631,251, 5,612,370, 5,612,323, 5,574,037, 5,563,170, 5,552,411, 5,552,397, 5,547,966, 5,482,925, 5,457,118, 5,414,017, 5,414,013, 5,401,758, 5,393,771, 5,362,902, 5,332,737, 5,310,731, 5,260,444, 5,223,516, 5,217,958, 5,208,245, 5,202,330, 5,198,449,5,189,036, 5,185,362, 5,140,031, 5,128,349, 5,116,861, 5,079,247, 5,070,099, 5,061,813, 5,055,466, 5,051,423, 5,036,065, 5,026,712, 5,011,931, 5,006,542, 4,981,843, 4,977,144, 4,971,984, 4,966,967, 4,959,383, 4,954,526, 4,952,692, 4,939,137, 4,906,634, 4,889,866, 4,888,335, 4,883,872, 4,883,811, 4,847,379, 4,835,157, 4,824,831, 4,780,538, 4,775,757, 4,774,239, 4,771,047, 4,769,371, 4,767,756, 4,762,837, 4,753,946, 4,752,616, 4,749,715, 4,738,978, 4,735,962, 4,734,426, 4,734,425, 4,734,424, 4,730,052, 4,727,072, 4,721,796, 4,707,550, 4,704,382, 4,703,120, 4,681,970, 4,681,882, 4,670,560, 4,670,453, 4,668,787, 4,663,337, 4,663,336, 4,661,506, 4,656,267, 4,656,185, 4,654,357, 4,654,356, 4,654,355, 4,654,335, 4,652,578, 4,652,576, 4,650,874, 4,650,797, 4,649,139, 4,647,585, 4,647,573, 4,647,565, 4,647,561, 4,645,836, 4,639,461, 4,638,012, 4,638,011, 4,632,931, 4,631,283, 4,628,095, 4,626,548, 4,614,825, 4,611,007, 4,611,006, 4,611,005, 4,609,671, 4,608,386,4,607,049, 4,607,048, 4,595,692, 4,593,042, 4,593,029, 4,591,603, 4,588,743, 4,588,742, 4,588,741, 4,582,854, 4,575,512, 4,568,762, 4,560,698, 4,556,739, 4,556,675, 4,555,571,4,555,570, 4,555,523, 4,550,120, 4,542,160, 4,542,157, 4,542,156, 4,542,155, 4,542,151, 4,537,981, 4,537,904, 4,536,514, 4,536,513, 4,533,673, 4,526,901, 4,526,900, 4,525,479, 4,524,151, 4,522,949, 4,521,539, 4,520,026, 4,517,188, 4,482,562, 4,474,804, 4,474,803, 4,472,411, 4,466,979, 4,463,015, 4,456,617, 4,456,616, 4,456,615, 4,418,076, 4,416,896, 4,252,815, 4,220,594, 4,190,587, 4,177,280, 4,164,586, 4,151,297, 4,145,443, 4,143,054,4,123,550, 4,083,968, 4,076,834, 4,064,259, 4,064,258, 4,064,257, 4,058,620, 4,001,421, 3,993,639, 3,991,057, 3,982,010, 3,980,652, 3,968,117, 3,959,296, 3,951,950, 3,933,834, 3,925,369, 3,923,818, 3,898,210, 3,897,442, 3,897,441, 3,886,157, 3,883,540, 3,873,715, 3,867,383, 3,873,715, 3,867,383, 3,691,216, 3,624,126;
antimicrobial agents as disclosed in U.S. Pat. Nos. 5,902,594, 5,874,476, 5,874,436, 5,859,027, 5,856,320, 5,854,242, 5,811,091, 5,786,350, 5,783,177, 5,773,469, 5,762,919, 5,753,715, 5,741,526, 5,709,870, 5,707,990, 5,696,117, 5,684,042, 5,683,709, 5,656,591, 5,643,971, 5,643,950, 5,610,196, 5,608,056, 5,604,262, 5,595,742, 5,576,341, 5,554,373, 3,541,233, 5,534,546, 5,534,508, 5,514,715, 5,508,417, 5,464,832, 5,428,073, 5,428,016, 5,424,396, 5,399,553, 5,391,544, 5,385,902, 5,359,066, 5,356,803, 5,354,862, 5,346,913, 5,302,592, 5,288,693, 5,266,567, 5,254,685, 5,252,745, 5,209,930, 5,196,441, 5,190,961, 5,175,160, 5,157,051, 5,096,700, 5,093,342, 5,089,251, 5,073,570, 5,061,702, 5,037,809, 5,036,077, 5,010,109,4,970,226, 4,916,156, 4,888,434, 4,870,093, 4,855,318, 4,784,991, 4,746,504, 4,686,221, 4,599,228, 4,552,882, 4,492,700, 4,489,098, 4,489,085, 4,487,776, 4,479,953, 4,477,448,4,474,807, 4,470,994, 4,370,484, 4,337,199, 4,311,709, 4,308,283, 4,304,910, 4,260,634,4,233,311, 4,215,131, 4,166,122, 4,141,981, 4,130,664, 4,089,977, 4,089,900, 4,069,341, 4,055,655, 4,049,665, 4,044,139, 4,002,775, 3,991,201, 3,966,968, 3,954,868, 3,936,393, 3,917,476, 3,915,889, 3,867,548, 3,865,748, 3,867,548, 3,865,748, 3,783,160, 3,764,676, 3,764,677;
anti-inflammatory agents as disclosed in U.S. Pat. Nos. 5,872,109, 5,837,735, 5,827,837, 5,821,250, 5,814,648, 5,780,026, 5,776,946, 5,760,002, 5,750,543, 5,741,798, 5,739,279, 5,733,939, 5,723,481, 5,716,967, 5,688,949, 5,686,488, 5,686,471, 5,686,434, 5,684,204, 5,684,041, 5,684,031, 5,684,002, 5,677,318, 5,674,891, 5,672,620, 5,665,752, 5,656,661, 5,635,516, 5,631,283, 5,622,948, 5,618,835, 5,607,959, 5,593,980, 5,593,960, 5,580,888, 5,552,424, 5,552,422, 5,516,764, 5,510,361, 5,508,026, 5,500,417, 5,498,405, 5,494,927, 5,476,876, 5,472,973, 5,470,885, 5,470,842, 5,464,856, 5,464,849, 5,462,952, 5,459,151, 5,451,686, 5,444,043, 5,436,265, 5,432,181, RE034918, U.S. Pat. Nos. 5,393,756, 5,380,738, 5,376,670, 5,360,811, 5,354,768, 5,348,957, 5,347,029, 5,340,815, 5,338,753, 5,324,648, 5,319,099, 5,318,971, 5,312,821, 5,302,597, 5,298,633, 5,298,522, 5,298,498, 5,290,800, 5,290,788, 5,284,949, 5,280,045, 5,270,319, 5,266,562, 5,256,680, 5,250,700, 5,250,552, 5,248,682, 5,244,917, 5,240,929, 5,234,939, 5,234,937, 5,232,939, 5,225,571, 5,225,418, 5,220,025, 5,212,189, 5,212,172, 5,208,250, 5,204,365, 5,202,350, 5,196,431, 5,191,084, 5,187,175, 5,185,326, 5,183,906, 5,177,079, 5,171,864, 5,169,963, 5,155,122, 5,143,929, 5,143,928, 5,143,927, 5,124,455, 5,124,347, 5,114,958, 5,112,846, 5,104,656, 5,098,613, 5,095,037, 5,095,019, 5,086,064, 5,081,261, 5,081,147, 5,081,126, 5,075,330, 5,066,668, 5,059,602, 5,043,457, 5,037,835, 5,037,811, 5,036,088, 5,013,850, 5,013,751, 5,013,736, 5,006,542, 4,992,448, 4,992,447, 4,988,733, 4,988,728, 4,981,865, 4,962,119, 4,959,378, 4,954,519, 4,945,099, 4,942,236, 4,931,457, 4,927,835, 4,912,248, 4,910,192, 4,904,786, 4,904,685, 4,904,674, 4,904,671, 4,897,397, 4,895,953, 4,891,370, 4,870,210, 4,859,686, 4,857,644, 4,853,392, 4,851,412, 4,847,303, 4,847,290, 4,845,242, 4,835,166, 4,826,990, 4,803,216, 4,801,598, 4,791,129, 4,788,205, 4,778,818, 4,775,679, 4,772,703, 4,767,776, 4,764,525, 4,760,051, 4,748,153, 4,725,616, 4,721,712, 4,713,393, 4,708,966, 4,695,571, 4,686,235, 4,686,224, 4,680,298, 4,678,802, 4,652,564, 4,644,005, 4,632,923, 4,629,793, 4,614,741, 4,599,360, 4,596,828, 4,595,694, 4,595,686, 4,594,357, 4,585,755, 4,579,866, 4,578,390, 4,569,942, 4,567,201, 4,563,476, 4,559,348, 4,558,067, 4,556,672, 4,556,669, 4,539,326, 4,537,903, 4,536,503, 4,518,608, 4,514,415, 4,512,990, 4,501,755, 4,495,197, 4,493,839, 4,465,687, 4,440,779, 4,440,763, 4,435,420, 4,412,995, 4,400,534, 4,355,034, 4,335,141, 4,322,420, 4,275,064, 4,244,963, 4,235,908, 4,234,593, 4,226,887, 4,201,778, 4,181,720, 4,173,650, 4,173,634, 4,145,444, 4,128,664, 4,125,612, 4,124,726, 4,124,707, 4,117,135,4,027,031, 4,024,284,4,021,553, 4,021,550,4,018,923, 4,012,527,4,011,326, 3,998,970, 3,998,954, 3,993,763, 3,991,212, 3,984,405, 3,978,227, 3,978,219, 3,978,202, 3,975,543, 3,968,224, 3,959,368, 3,949,082, 3,949,081, 3,947,475, 3,936,450, 3,934,018, 3,930,005, 3,857,955, 3,856,962, 3,821,377, 3,821,401, 3,789,121, 3,789,123, 3,726,978, 3,694,471, 3,691,214, 3,678,169, 3,624,216;
immunosuppressive agents, as disclosed in U.S. Pat. Nos. 4,450,159, 4,450,159, 5,905,085, 5,883,119, 5,880,280, 5,877,184, 5,874,594, 5,843,452, 5,817,672, 5,817,661, 5,817,660, 5,801,193, 5,776,974, 5,763,478, 5,739,169, 5,723,466, 5,719,176, 5,696,156, 5,695,753, 5,693,648, 5,693,645, 5,691,346, 5,686,469, 5,686,424, 5,679,705, 5,679,640, 5,670,504, 5,665,774, 5,665,772, 5,648,376, 5,639,455, 5,633,277, 5,624,930, 5,622,970, 5,605,903, 5,604,229, 5,574,041, 5,565,560, 5,550,233, 5,545,734, 5,540,931, 5,532,248, 5,527,820, 5,516,797, 5,514,688, 5,512,687, 5,506,233, 5,506,228, 5,494,895, 5,484,788, 5,470,857, 5,464,615, 5,432,183, 5,431,896, 5,385,918, 5,349,061, 5,344,925, 5,330,993, 5,308,837, 5,290,783, 5,290,772, 5,284,877, 5,284,840, 5,273,979, 5,262,533, 5,260,300, 5,252,732, 5,250,678, 5,247,076, 5,244,896, 5,238,689, 5,219,884, 5,208,241, 5,208,228, 5,202,332, 5,192,773, 5,189,042, 5,169,851, 5,162,334, 5,151,413, 5,149,701, 5,147,877, 5,143,918, 5,138,051, 5,093,338, 5,091,389, 5,068,323, 5,068,247, 5,064,835, 5,061,728, 5,055,290, 4,981,792, 4,810,692, 4,410,696, 4,346,096, 4,342,769, 4,317,825, 4,256,766, 4,180,588, 4,000,275, 3,759,921;
analgesic agents, as disclosed in U.S. Pat. Nos. 5,292,736, 5,688,825, 5,554,789, 5,455,230, 5,292,736, 5,298,522, 5,216,165, 5,438,064, 5,204,365, 5,017,578, 4,906,655, 4,906,655, 4,994,450, 4,749,792, 4,980,365, 4,794,110, 4,670,541, 4,737,493, 4,622,326, 4,536,512, 4,719,231, 4,533,671, 4,552,866, 4,539,312, 4,569,942, 4,681,879, 4,511,724, 4,556,672, 4,721,712, 4,474,806, 4,595,686, 4,440,779, 4,434,175, 4,608,374, 4,395,402, 4,400,534, 4,374,139, 4,361,583, 4,252,816, 4,251,530, 5,874,459, 5,688,825, 5,554,789, 5,455,230, 5,438,064, 5,298,522, 5,216,165, 5,204,365, 5,030,639, 5,017,578, 5,008,264, 4,994,450, 4,980,365, 4,906,655, 4,847,290, 4,844,907, 4,794,110, 4,791,129, 4,774,256, 4,749,792, 4,737,493, 4,721,712, 4,719,231, 4,681,879, 4,670,541, 4,667,039, 4,658,037, 4,634,708, 4,623,648, 4,622,326, 4,608,374, 4,595,686, 4,594,188, 4,569,942, 4,556,672, 4,552,866, 4,539,312, 4,536,512, 4,533,671, 4,511,724, 4,440,779, 4,434,175, 4,400,534, 4,395,402, 4,391,827, 4,374,139, 4,361,583, 4,322,420, 4,306,097, 4,252,816, 4,251,530, 4,244,955, 4,232,018, 4,209,520, 4,164,514, 4,147,872, 4,133,819, 4,124,713, 4,117,012, 4,064,272, 4,022,836, 3,966,944;
cholinergic agents, as disclosed in U.S. Pat. Nos. 5,219,872, 5,219,873, 5,073,560, 5,073,560, 5,346,911, 5,424,301, 5,073,560, 5,219,872, 4,900,748, 4,786,648, 4,798,841, 4,782,071, 4,710,508, 5,482,938, 5,464,842, 5,378,723, 5,346,911, 5,318,978, 5,219,873, 5,219,872, 5,084,281, 5,073,560, 5,002,955, 4,988,710, 4,900,748, 4,798,841, 4,786,648, 4,782,071, 4,745,123, 4,710,508;
adrenergic agents, as disclosed in U.S. Pat. Nos. 5,091,528, 5,091,528, 4,835,157, 5,708,015, 5,594,027, 5,580,892, 5,576,332, 5,510,376, 5,482,961, 5,334,601, 5,202,347, 5,135,926, 5,116,867, 5,091,528, 5,017,618, 4,835,157, 4,829,086, 4,579,867, 4,568,679, 4,469,690, 4,395,559, 4,381,309, 4,363,808, 4,343,800, 4,329,289, 4,314,943, 4,311,708, 4,304,721, 4,296,117, 4,285,873, 4,281,189, 4,278,608, 4,247,710, 4,145,550, 4,145,425, 4,139,535, 4,082,843, 4,011,321, 4,001,421, 3,982,010, 3,940,407, 3,852,468, 3,832,470;
antihistamine agents, as disclosed in U.S. Pat. Nos. 5,874,479, 5,863,938, 5,856,364, 5,770,612, 5,702,688, 5,674,912, 5,663,208, 5,658,957, 5,652,274, 5,648,380, 5,646,190, 5,641,814, 5,633,285, 5,614,561, 5,602,183, 4,923,892, 4,782,058, 4,393,210, 4,180,583, 3,965,257, 3,946,022, 3,931,197;
steroidal agents, as disclosed in U.S. Pat. Nos. 5,863,538, 5,855,907, 5,855,866, 5,780,592, 5,776,427, 5,651,987, 5,346,887, 5,256,408, 5,252,319, 5,209,926, 4,996,335, 4,927,807, 4,910,192, 4,710,495, 4,049,805, 4,004,005, 3,670,079, 3,608,076, 5,892,028, 5,888,995, 5,883,087, 5,880,115, 5,869,475, 5,866,558, 5,861,390, 5,861,388, 5,854,235, 5,837,698, 5,834,452, 5,830,886, 5,792,758, 5,792,757, 5,763,361, 5,744,462, 5,741,787, 5,741,786, 5,733,899, 5,731,345, 5,723,638, 5,721,226, 5,712,264, 5,712,263, 5,710,144, 5,707,984, 5,705,494, 5,700,793, 5,698,720, 5,698,545, 5,696,106, 5,677,293, 5,674,861, 5,661,141, 5,656,621, 5,646,136, 5,637,691, 5,616,574, 5,614,514, 5,604,215, 5,604,213, 5,599,807, 5,585,482, 5,565,588, 5,563,259, 5,563,131, 5,561,124, 5,556,845, 5,547,949, 5,536,714, 5,527,806, 5,506,354, 5,506,221, 5,494,907, 5,491,136, 5,478,956, 5,426,179, 5,422,262, 5,391,776, 5,382,661, 5,380,841, 5,380,840, 5,380,839, 5,373,095, 5,371,078, 5,352,809, 5,344,827, 5,344,826, 5,338,837, 5,336,686, 5,292,906, 5,292,878, 5,281,587, 5,272,140, 5,244,886, 5,236,912, 5,232,915, 5,219,879, 5,218,109, 5,215,972, 5,212,166, 5,206,415, 5,194,602, 5,166,201, 5,166,055, 5,126,488, 5,116,829, 5,108,996, 5,099,037, 5,096,892, 5,093,502, 5,086,047, 5,084,450, 5,082,835, 5,081,114, 5,053,404, 5,041,433, 5,041,432, 5,034,548, 5,032,586, 5,026,882, 4,996,335, 4,975,537, 4,970,205, 4,954,446, 4,950,428, 4,946,834, 4,937,237, 4,921,846, 4,920,099, 4,910,226, 4,900,725, 4,892,867, 4,888,336, 4,885,280, 4,882,322, 4,882,319, 4,882,315, 4,874,855, 4,868,167, 4,865,767, 4,861,875, 4,861,765, 4,861,763, 4,847,014, 4,774,236, 4,753,932, 4,711,856, 4,710,495, 4,701,450, 4,701,449, 4,689,410, 4,680,290, 4,670,551, 4,664,850, 4,659,516, 4,647,410, 4,634,695, 4,634,693, 4,588,530, 4,567,000, 4,560,557, 4,558,041, 4,552,871, 4,552,868, 4,541,956, 4,519,946, 4,515,787, 4,512,986, 4,502,989, 4,495,102;
   the disclosures of all the above of which are herein incorporated by reference.

The drug moiety of the bioconjugate may be the whole compound or a binding fragment or portion thereof that retains its affinity and specificity for the target of interest while having a linkage site for covalent bonding to the presenter protein ligand or linker.

Biopolymers According to the Invention

The biopolymer may comprise a naturally occurring or modified protein, peptide, polynucleic acid, or polysaccharide. The biopolymer is preferably a naturally occurring protein. The protein may be any protein that is capable of delivering the active agent to a target site or compartment. In a preferred embodiment, the protein is a membrane transport protein which is capable of translocating itself and/or a bound entity from the extracellular surface to the intracellular compartment. In another embodiment, the protein is a membrane transport protein which can transport a bound entity across the blood brain barrier. In another embodiment, the compartment is a CNS or CSF compartment. In another embodiment, the protein is a transport protein which binds to a particular tissue or cell type.

In another embodiment, the biopolymer is an antibody directed toward a protein that is capable of delivering the active agent to a target site or compartment as discussed above. In another embodiment, the biopolymer is an antibody to a tumor or disease associated antigen or a cell surface marker.

In one embodiment, the biopolymer comprises p97 or a substance which is capable of specifically binding to p97, such as an antibody to p97. In a further embodiment, the agent may be a substance having therapeutic activity such as a growth factor or lymphokine, enzyme or drug. The invention also relates to a method of delivering an active agent across the blood brain barrier comprising administering a bioconjugate of Formula I, wherein the biopolymer comprises p97 or an antibody to p97 or a p97 protein portion or fragment with p97 transport activity. In one embodiment, the p97 protein is soluble. P97 proteins as taught in U.S. Pat. No. 5,981,194 are particularly preferred. The p97 may be a human p97 protein or fragment thereof; the p97 may be from a mammal such as a mouse. Murine p97 is disclosed in WO 01/59549 A2 which is herein incorporated by reference in its entirety.

“p97” as used in the compositions of the invention, includes membrane bound p97 (i.e., p97 linked to GPI or other lipids), soluble p97, cleaved p97, analogs of p97 which are equivalents of p97 (having greater than 40%, 60%, 80%, or 90% homology at the peptide sequence level, including allelic variants of p97), human, mouse, chicken and/or rabbit p97, and derivatives, portions, or fragments thereof. p97 may be in the form of acidic or basic salts, or in neutral form. In addition, individual amino acid residues may be modified, such as by oxidation or reduction. Various substitutions, deletions, or additions may be made to the amino acid or DNA nucleic acid sequences, the net effect of which is to retain or improve upon the desired biological activity of p97. Due to code degeneracy, for example, there may be considerable variation in nucleotide sequences encoding the same amino acid sequence. As used herein, p97 also includes fragments of p97, including any portion of p97 or its biologically equivalent analogs that contain a sufficient portion of p97 and homology to the corresponding native p97 amino acid sequence to enable it to retain or improve upon the desired biological activities of p97. In other aspects, the invention is drawn to p97 bioconjugates which have only minor substitutions in the amino acid sequence which do not substantially affect its receptor binding or transcytosis properties.

Preferred chemotherapeutic agents for use in p97-chemotherapeutic agents of the invention include all drugs which may be useful for treating brain tumors or other neoplasia in or around the brain, either in the free form, or, if not so useful in the free form, then useful when linked to p97. Such chemotherapeutic agents include adriamycin (also known as doxorubicin), cisplatin, paclitaxel, analogs thereof, and other chemotherapeutic agents which demonstrate activity against tumors ex vivo and in vivo. Such chemotherapeutic agents also include alkylating agents, antimetabolites, natural products (such as vinca alkaloids, epidophyllotoxins, antibiotics, enzymes and biological response modifiers), topoisomerase inhibitors, microtubule inhibitors, spindle poisons, hormones and antagonists, and miscellaneous agents such as platinum coordination complexes, anthracendiones, substituted ureas, etc. those of skill in the art will know of other chemotherapeutic agents.

p97-chemotherapeutic agents can comprise one or more compound moieties linked to p97. For example, conjugation reactions may conjugate from 1 to 10 or more molecules of adriamycin to a single p97 molecule. Several atoms of gold or iodine can be conjugated to a single p97 polypeptide. These formulations can be employed as mixtures, or they may be purified into specific p97:compound (mol:mol) formulations. Those skilled in the art are able to determine which format and which mol:mol ratio is preferred. Further, mixtures of compounds may be linked to p97, such as the p97-adriamycin-cisplatinum composition set out in the examples. These p97-chemotherapeutic agents may consist of a range of mol:mol ratios. These, too, may be separated into purified mixtures or they may be employed in aggregate.

The compositions of the invention may also be used for delivering an agent across the blood eye barrier or blood placenta barrier

Labels

In some embodiments, the bioconjugate is labeled to facilitate its detection. A “label” or a “detectable moiety” is a composition detectable by spectroscopic, photochemical, biochemical, immunochemical, chemical, or other physical means. For example, labels suitable for use in the present invention include, for example, radioactive labels (e.g., ³²P), fluorophores (e.g., fluorescein), electron-dense reagents, enzymes (e.g., as commonly used in an ELISA), biotin, digoxigenin, or haptens and proteins which can be made detectable, e.g., by incorporating a radiolabel into the hapten or peptide, or used to detect antibodies specifically reactive with the hapten or peptide.

As noted above, depending on the screening assay employed, the drug, the linker or the biopolymer portion of a bioconjugate may be labeled. The particular label or detectable group used is not a critical aspect of the invention, as long as it does not significantly interfere with the biological activity of the bioconjugate. The detectable group can be any material having a detectable physical or chemical property. Thus, a label is any composition detectable by spectroscopic, photochemical, biochemical, immunochemical, electrical, optical or chemical means.

Examples of labels suitable for use in the present invention include, but are not limited to, fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. Preferably, the label in one embodiment is covalently bound to the biopolymer using an isocyanate reagent for conjugating an active agent according to the invention. In one aspect of the invention, the bifunctional isocyanate reagents of the invention can be used to conjugate a label to a biopolymer to form a label biopolymer conjugate without an active agent attached thereto. The label biopolymer conjugate may be used as an intermediate for the synthesis of a labeled bioconjugate according to the invention or may be used to detect the biopolymer conjugate. As indicated above, a wide variety of labels can be used, with the choice of label depending on sensitivity required, ease of conjugation with the desired component of the assay, stability requirements, available instrumentation, and disposal provisions. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound.

The bioconjugates can also be conjugated directly to signal generating compounds, e.g., by conjugation with an enzyme or fluorophore. Enzymes suitable for use as labels include, but are not limited to, hydrolases, particularly phosphatases, esterases and glycosidases, or oxidotases, particularly peroxidases. Fluorescent compounds, i.e., fluorophores, suitable for use as labels include, but are not limited to, fluorescein and its derivatives, rhodamine and its derivatives, dansyl, umbelliferone, etc. Further examples of suitable fluorophores include, but are not limited to, eosin, TRITC-amine, quinine, fluorescein W, acridine yellow, lissamine rhodamine, B sulfonyl chloride erythroscein, ruthenium (tris, bipyridinium), Texas Red, nicotinamide adenine dinucleotide, flavin adenine dinucleotide, etc. Chemiluminescent compounds suitable for use as labels include, but are not limited to, luciferin and 2,3-dihydrophthalazinediones, e.g., luminol. For a review of various labeling or signal producing systems that can be used in the methods of the present invention, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Other labeling and detection systems suitable for use in the methods of the present invention will be readily apparent to those of skill in the art.

Isocyanate Reagents According to the Invention

The bifunctional cross linking reagents comprises at least two reactive groups, at least one of which is an isocyanate functional group. In one embodiment, the bifunctional cross linking reagent is a diisocyanate of the following formula: 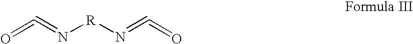
in which R is unsubstituted alkylene or substituted alkyl or unsubstituted or substituted heteroalkylene from 1 to about 30 atoms in length or from about 30 to about 50 atoms in length.

In another embodiment, the bifunctional cross linking reagent is an isocyanate of the following formula: 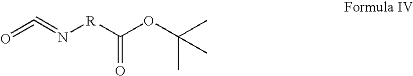
in which R is substituted alkylene or unsubstituted alkylene or unsubstituted or substituted heteroalkylene from 1 to about 30 atoms, or from 30 to about 50 atoms in length. Other blocking groups than the t-butyl group would be obvious to one of ordinary skill in the art.

The term “protecting group” or “compatible protecting group” refers to a chemical group that exhibits the following characteristics: 1) reacts selectively with the desired functionality in good yield to give a derivative that is stable to the projected reactions for which protection is desired; 2) can be selectively removed chemically and/or enzymatically from the derivatized solid support to yield the desired functionality; and 3) is removable in good yield by reagents compatible with the other functional group(s) generated in such projected reactions. Examples of protecting groups can be found in Greene, et al. (1991) Protective Groups in Organic Synthesis, 2nd Ed. (John Wiley & Sons, Inc., New York). Preferred protecting groups include, but are not limited to, acid-labile protecting groups (such as Boc or DMT); base-labile protecting groups (such as Fmoc, Fm, phosphonioethoxycarbonyl (Peoc), etc.); groups which may be removed under neutral conditions (e.g., metal ion-assisted hydrolysis), such as DBMB, allyl or alloc, 2-haloethyl; groups which may be removed using fluoride ion, such as 2-(trimethylsilyl)ethoxymethyl (SEM), 2-(trimethylsilyl)-ethyloxycarbonyl (Teoc) or 2-(trimethylsilyl)ethyl (Te) S; and groups which may be removed under mild reducing conditions (e.g., with sodium borohydride or hydrazine), such as Lev. Particularly preferred protecting groups include, but are not limited to, Fmoc, Fm, Menpoc, Nvoc, Nv, Boc, CBZ, allyl, alloc (allyloxycarbonyl), Npeoc (4-nitrophenethyloxycarbonyl), Npeom (4-nitrophenethyloxymethyloxy), α,α-dimethyl-3,5-dimethoxybenzyloxycarbonyl (ddz) and trityl groups. The particular removable protecting group employed is not critical to the methods of the present invention.

The term “orthogonal protecting groups” refer to two or more compatible protecting groups which, in the presence of one other, can be differentially removed or, if not differentially removed, can be differentially reprotected. In one embodiment, it may be desirable to remove all of the protecting groups in one step, such as at completion of the synthesis.

The term “alkyl,” by itself or as part of another substituent, means is as defined above.

Where an alkane or alkyl or alkylene member is designated as R in Formula I, for instance, the corresponding divalent alkyl radical is indicated. For example, where R is designated as methane, methyl, or methylene; the corresponding compounds of Formula III or IV would be

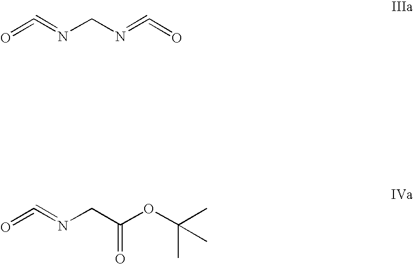

In one embodiment, the cross-linking reagent is O═C=N(CH₂)ₙNHC(O)(OCH₂CH₂)ₘ(CH₂)ₚC(O)OC(CH₃)₃ wherein n is from about 2 to 12, m is about 2 to 30, and p is about 1 to 12. In a preferred embodiment, n is 6, m is 5, and p is 2: 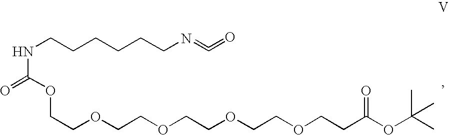
Methods of Using Pharmaceutical Compositions, and their Administration

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent. Typical modes of administration are described below.

The term “effective amount” means a dosage sufficient to produce a desired result on a health condition, pathology, or disease of a subject. The desired result may comprise a subjective or objective improvement in the recipient of the dosage.

A “prophylactic treatment” is a treatment administered to a subject who does not exhibit signs of a disease or exhibits only early signs of a disease, wherein treatment is administered for the purpose of decreasing the risk of developing a pathological condition. The bioconjugate compounds of the invention may be given as a prophylactic treatment.

A “therapeutic treatment” is a treatment administered to a subject who exhibits signs of pathology, wherein treatment is administered for the purpose of diminishing or eliminating those pathological signs. The bioconjugate compounds of the invention may be given as a prophylactic treatment.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a bioconjugate compound of the present invention and a pharmaceutically acceptable carrier. The term “pharmaceutical composition” indicates a composition suitable for pharmaceutical use in a subject, including an animal or human. A pharmaceutical composition generally comprises an effective amount of a bioconjugate and a pharmaceutically acceptable carrier.

The bioconjugates may be administered by a variety of routes. For oral preparations, the bioconjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bioconjugates can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The bioconjugates can be utilized in aerosol formulation to be administered via inhalation. The compounds of the present invention can be formulated into pressurized acceptable propellants such as dichlorodifluoromethane, propane, nitrogen and the like.

Furthermore, the bioconjugates can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms of the bioconjugate for oral or rectal administration such as syrups, elixirs, and suspensions may be provided wherein each dosage unit, for example, teaspoonful, tablespoonful, tablet or suppository, contains a predetermined amount of the composition containing active agent. Similarly, unit dosage forms for injection or intravenous administration may comprise the bioconjugate in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular bioconjugate employed and the effect to be achieved, and the pharmacodynamics associated with each compound in the host.

In practical use, the bioconjugates according to the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

With respect to transdermal routes of administration, methods for transdermal administration of drugs are disclosed in Remington's Pharmaceutical Sciences, 17th Edition, (Gennaro et al. Eds., Mack Publishing Co., 1985). Dermal or skin patches are a preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012, 6,261,595, and 6,261,595. Each of which is incorporated by reference in its entirety.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In each of these aspects, the compositions include, but are not limited to, compositions suitable for oral, rectal, topical, parenteral (including subcutaneous, intramuscular, and intravenous), pulmonary (nasal or buccal inhalation), or nasal administration, although the most suitable route in any given case will depend in part on the nature and severity of the conditions being treated and on the nature of the active ingredient. Exemplary routes of administration are the oral and intravenous routes. The compositions may be conveniently presented in unit dosage form and prepared by any of the methods well-known in the art of pharmacy.

In practical use, the compounds according to the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. The percentage of an active compound in these compositions may, of course, be varied and may conveniently be between about 2 percent to about 60 percent of the weight of the unit.

The bioconjugates of the invention are useful for therapeutic, prophylactic and diagnostic intervention in animals, and in particular in humans. As described herein, the bioconjugates show preferential accumulation and/or release of the active agent in any target organ, compartment, or site depending upon the biopolymer used.

Compositions of the present invention may be administered encapsulated in or attached to viral envelopes or vesicles, or incorporated into cells. Vesicles are micellular particles which are usually spherical and which are frequently lipidic. Liposomes are vesicles formed from a bilayer membrane. Suitable vesicles include, but are not limited to, unilamellar vesicles and multilamellar lipid vesicles or liposomes. Such vesicles and liposomes may be made from a wide range of lipid or phospholipid compounds, such as phosphatidylcholine, phosphatidic acid, phosphatidylserine, phosphatidylethanolamine, sphingomyelin, glycolipids, gangliosides, etc. using standard techniques, such as those described in, e.g., U.S. Pat. No. 4,394,448. Such vesicles or liposomes may be used to administer compounds intracellularly and to deliver compounds to the target organs. Controlled release of a p97-composition of interest may also be achieved using encapsulation (see, e.g., U.S. Pat. No. 5,186,941).

Any route of administration which dilutes the bioconjugate composition into the blood stream, or at least outside of the blood-brain barrier, may be used. Preferably, the composition is administered peripherally, most preferably intravenously or by cardiac catheter. Intra-jugular and intra-carotid injections are also useful. Compositions may be administered locally or regionally, such as intra-peritoneally. In one aspect, compositions are administered with a suitable pharmaceutical diluent or carrier.

Dosages to be administered will depend on individual needs, on the desired effect, the active agent used, the biopolymer and on the chosen route of administration. Preferred dosages of a bioconjugate range from about 0.2 pmol/kg to about 25 nmol/kg, and particularly preferred dosages range from 2-250 pmol/kg; alternatively, preferred doses of the bioconjugate may be in the range of 0.02 to 2000 mg/kg. These dosages will be influenced by the number of active agent or drug moieties associated with the biopolymer. Alternatively, dosages may be calculated based on the active agent administered.

In preferred embodiment the biopolymer is p97. For instance, doses of p97-adriamycin comprising from 0.005 to 100 mg/kg of adriamycin are also useful in vivo. Particularly preferred is a dosage of p97-adriamycin comprising from 0.05 mg/kg to 20 mg/kg of adriamycin. Those skilled in the art can determine suitable doses for other compounds linked to p97 based on the recommended dosage used for the free form of the compound. p97 generally reduces the amount of drug needed to obtain the same effect.

The p97-compounds of the invention are useful for therapeutic, prophylactic and diagnostic intervention in animals, and in particular in humans. As described herein, p97-compounds show preferential accumulation in the lung, liver, kidney and spleen, and that they significantly reduce delivery of the compounds to the heart. Preferred medical indications for diagnostic uses include, for example, any condition associated with a target organ of interest (e.g., lung, liver, kidney, spleen) or any condition that requires a cardiotoxic compound that would benefit by reducing its cardiotoxicity.

The subject methods find use in the treatment of a variety of different disease conditions. In certain embodiments, of particular interest is the use of the subject methods in disease conditions where an active agent or drug having desired activity has been previously identified, but in which the active agent or drug is not targeted to the target site, area or compartment. With such active agents or drugs, the subject methods can be used to enhance the therapeutic efficacy and therapeutic index of active agent or drug.

The specific disease conditions treatable by with the subject bioconjugates are as varied as the types of drug moieties that can be present in the bioconjugate. Thus, disease conditions include cellular proliferative diseases, such as neoplastic diseases, autoimmune diseases, cardiovascular diseases, hormonal abnormality diseases, degenerative diseases, diseases of aging, diseases of the central nervous system (e.g., Alzheimer's disease, epilepsy), psychiatric diseases and conditions (e.g., schizophrenia, mood disorders such as depression and anxiety), infectious diseases, and the like.

Treatment is meant to encompass any beneficial outcome to a subject associated with administration of a bioconjugate including a reduced likelihood of acquiring a disease, prevention of a disease, slowing, stopping or reversing, the progression of a disease or an amelioration of the symptoms associated with the disease condition afflicting the host, where amelioration or benefit is used in a broad sense to refer to at least a reduction in the magnitude of a parameter, e.g., symptom, associated with the pathological condition being treated, such as inflammation and pain associated therewith. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

A variety of hosts or subjects are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of the bioconjugate, usually in oral or injectable doses and often in a storage stable formulation, are provided. In such kits, in addition to the containers containing the unit doses will be an informational package insert describing the use and attendant benefits of the drugs in treating pathological condition of interest. Preferred compounds and unit doses are those described herein above.

### EXAMPLES

The following examples are provided by way of illustration only and not by way of limitation. Those of skill will readily recognize a variety of non-critical parameters which could be changed or modified to yield essentially similar results.

It has been found that isocyanate linkers are surprisingly very efficient reagents for the synthesis of bioconjugates including particularly small drug molecules to proteins, (e.g., p97). The linkers are particularly useful for any active agent which contain active hydroxy or amino groups. Such drugs, for example, are doxorubicin, taxol, camptothecin, SN-38, 10-hydroxycamptothecin, 7-(C₁-C₆alkyl) 10-hydroxycamptothecin, etc.

The compounds of the present invention can be made with commercially available starting materials. The following procedures are exemplary synthetic routes, which are intended to illustrate, but not to limit the present invention. With benefit thereof, one of ordinary skill in the art will recognize other variations, modifications, and alternatives of the disclosed methods.

### Example 1

Use of two exemplary bifunctional cross-linkers to form a bioconjugates of a drug containing hydroxy or amino groups with a biopolymer containing hydroxy or amino reactive coupling groups.

(A). Covalent coupling of drugs having hydroxy or amine functional groups with the exemplary cross-linking reagents of the present invention.

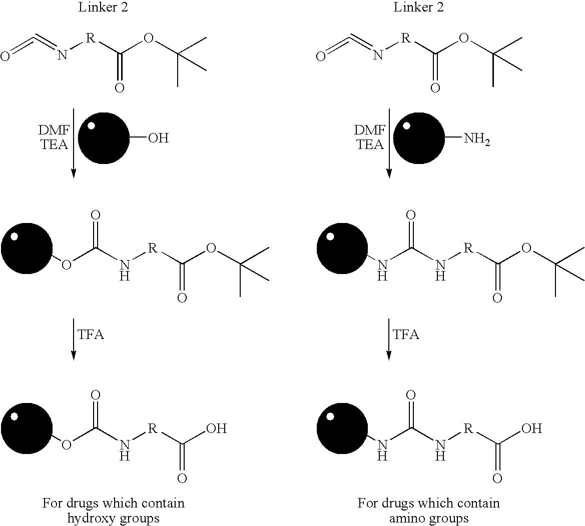

(B). Reacting the modified small drug with an exemplary biopolymer to generate the bioconjugate.

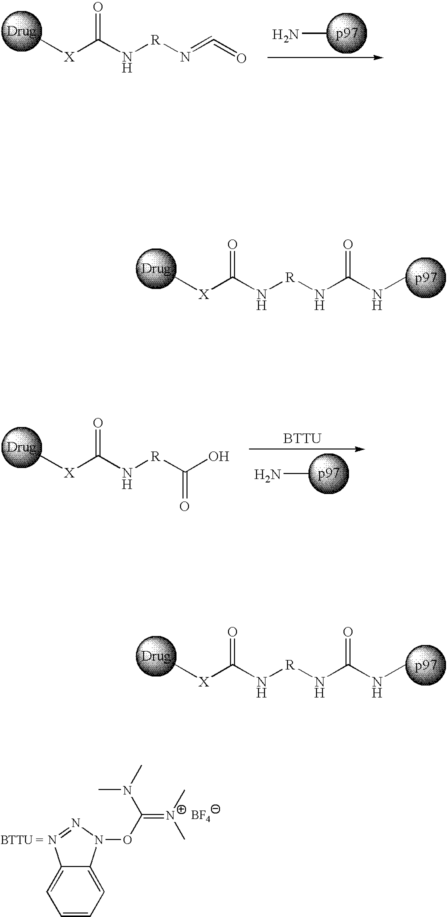

### Example 2

### Synthesis of Exemplary Isocyanate Linkers and their Usage for Modification of an Active Agent

Exemplary isocyanate linkers include, but are not limited to, those of the following formula:

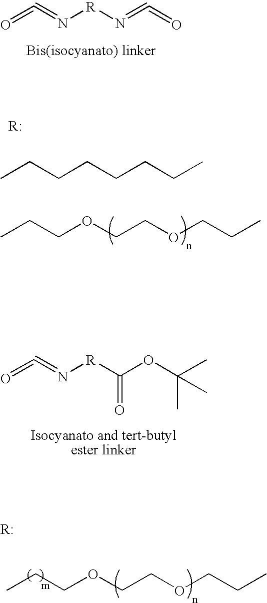

Synthesis of the Above Exemplary Linkers.

A. diisocyanate Linkers Synthesized from their Diacid Analogs: 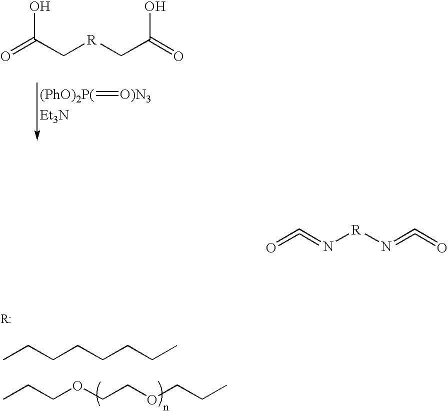
The starting diacid compounds are treated with diphenylphosphoryl azide to offer the expected diisocyanate linkers in high yield. The substituted group R could, for instance, be an alkyl group or PEG chains (n=3-10).

Synthesis of Isocyanate Tert-Butyl Ester Linker:

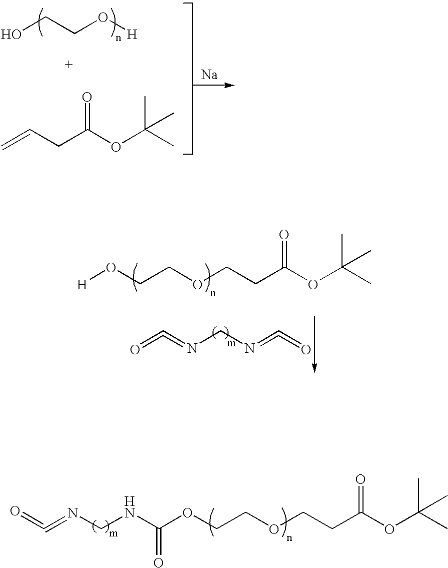

PEG (n=3-10) compounds are first reacted with tert-butyl acrylate to generate the mono tert-butyl ester, which is further reacted with diisocyanate compounds (m=4-6) to form the isocyanate tert-butyl ester linkers.

### Example 3

Examples of modifications of small drug molecules using the new linkers and their usage for small drug bioconjugate with p97

Diisocyanate Linker Conjugated 10-hydroxycamptothecin

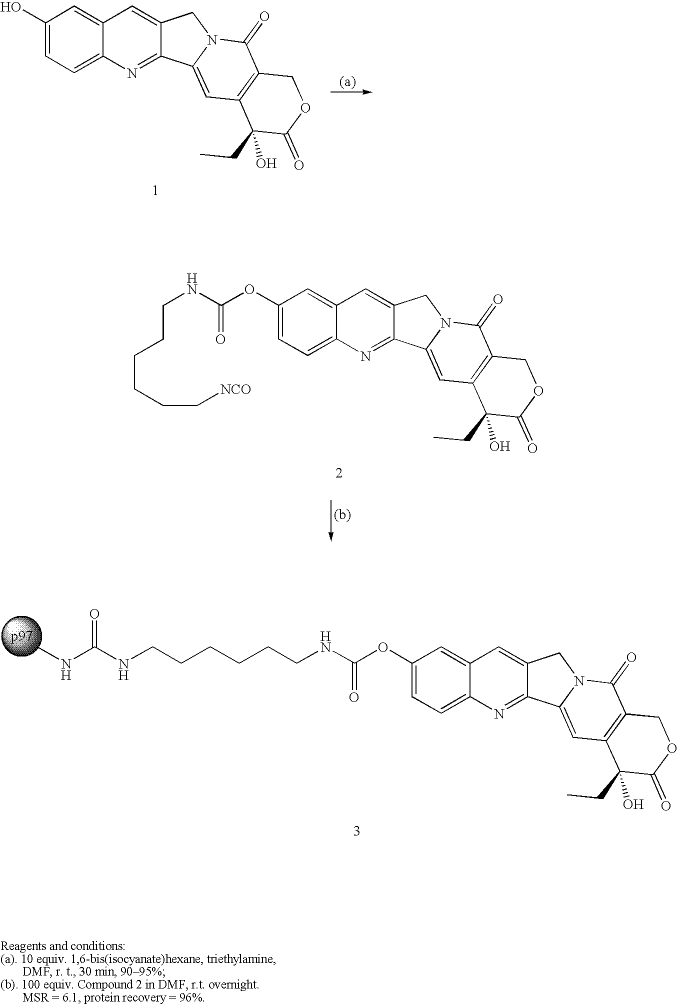

10-hydroxycamptothecin 1 is reacted with a diisocyanate linker to yield a mono isocyanate modified 10-hydroxycamptothecin in more than 90% yield. Simply mixing a solution of compound 2 in DMF with p97 will generate the expected conjugate 3 with MSR=6, protein recovery 96%.

Diisocyanate Linker Conjugated with SN-38:

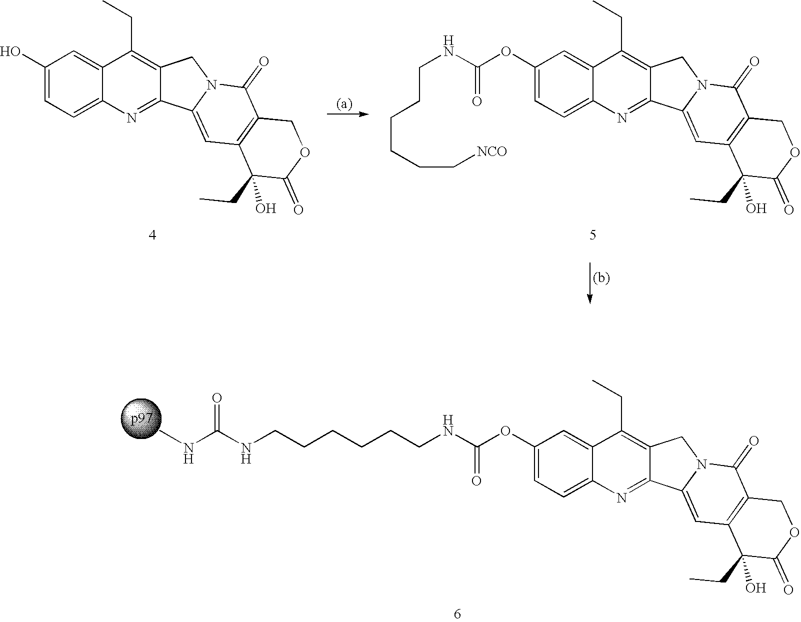

Reaction of Diisocyanate PEG Linker with SN-38 or Doxorubicin

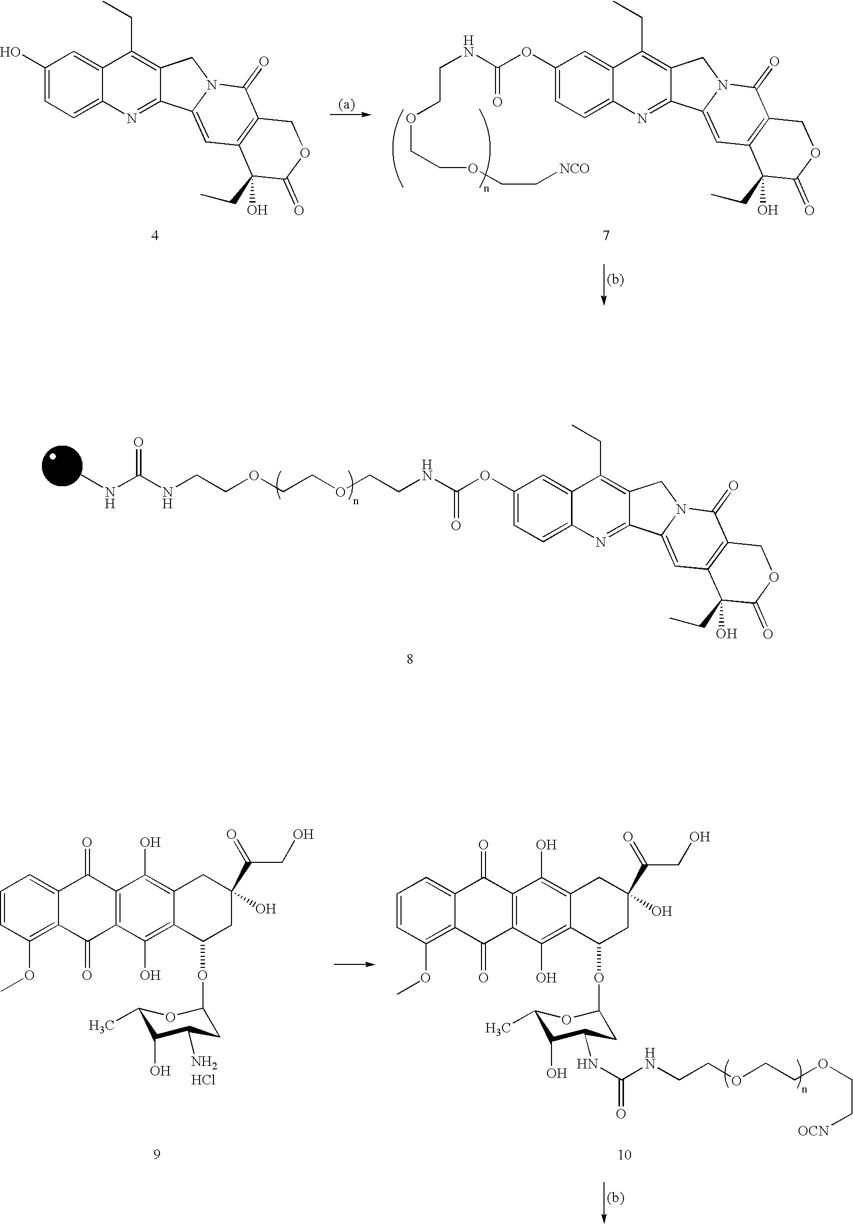

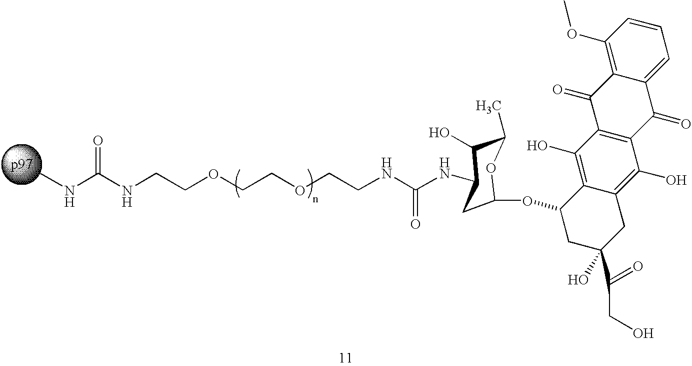

### Example 4

The reaction of Isocyanate Tert-Butyl Ester Linker with Taxol or SN-38

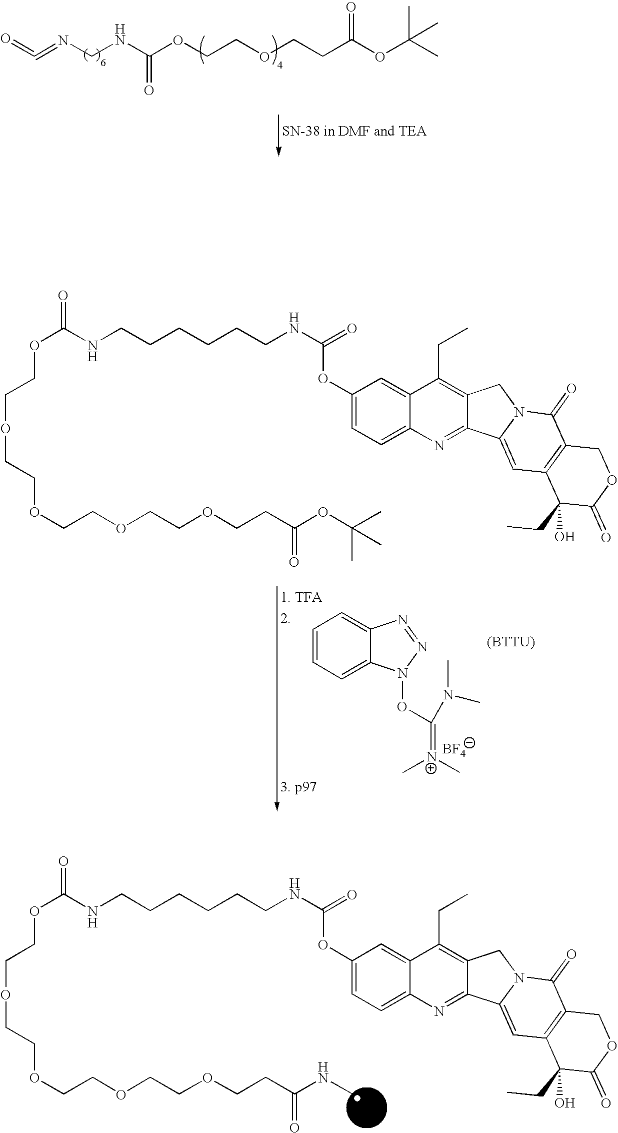

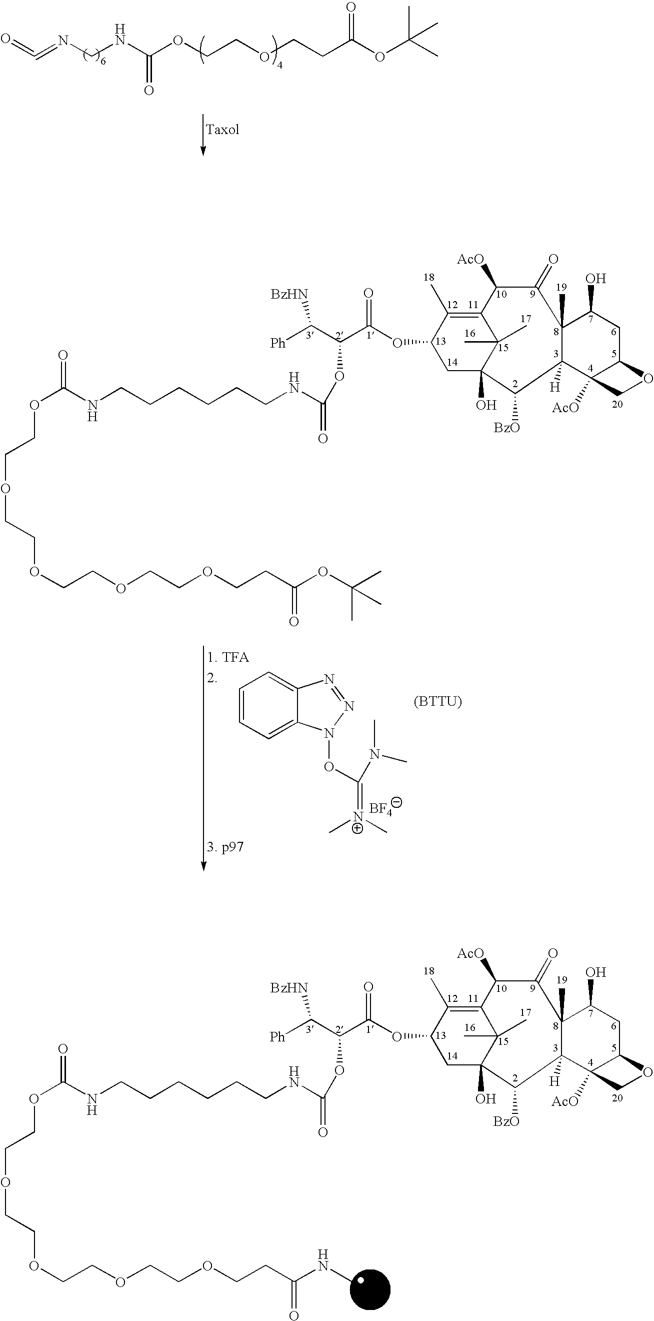

Experimental Procedures; Materials and Methods and Analytical Data

Camptothecin and 10-hydroxycamptothecin are available commercially (e.g., Abratra Technologies, Co. LTD (address: 78 Xiying Road Xi'an, 710054, China, Tel: 86-29-551-3489, Fax: 86-29-551-0486), email: info@abatra.com, abatra@yeah.net, http://www.abatra.com.).

Slider-A-Lyzer Dialysis Cassette, 10,000MWCO and SnakeSkin™ dialysis tubing, 10,000MWCO were purchased from Pierce Inc (3747 N. Meridian Road, P.O. Box 117, Rockford, Ill. USA 61105, http://wvw.piercenet.com). D-Salt™ Excellulose™ plastic desalting columns (type 20450, 5 mL, gel exclusion limit 5000 MW) was purchased from Pierce Inc. (3747 N. Meridian Road, P.O. Box 117, Rockford, Ill. 61105, www.piercenet.com). FPLC was recorded in a AKTA Purifier™0 FPLC (using UNICORN™ version 3.10, Amersham Pharmacia Biotech) using Mono Q^{R} HR 10/10 ion exchange column (from Pharmacia Biotech Inc.) and PBS buffer (0.01 M, pH=7.4) and 1M NaCl-0.001 M PBS buffers mobile phases or using BIOSEP™ size exclusion column (from Phenomenex, Inc., Torrance, Calif., USA) and 0.01 M PBS buffer (pH 6.80). All other chemical reagents and solvents were purchased from Aldrich, Sigma, or VWR and used as received. The silica gel used in flash chromatography was Merck silica gel 60, 230-400 mesh whilst R_{f} values were measured on Merck silica TLC aluminum sheets (silica gel 60 F₂₅₄). Melting points were determined on a Thomas hot stage or Buchi apparatus and are uncorrected. ¹H and ¹³C NMR spectra were recorded on Bruker AC-200 or AMX-300 instruments. UV-Vis spectra were recorded on an HP8452A photo diode array spectrophotometer (instrumental precision±2 nm) in the solvents indicated. Elemental analyses were performed by the microanalytical laboratory, Department of Chemistry, UBC. The high and low resolution mass spectra were obtained by mass spectrometer service laboratories, Department of Chemistry, UBC.

### 10-Hydroxycamptothecin 6-isocyanatehexylcarbamate [C₂₈H₂₈N₄O₇, FW=532]

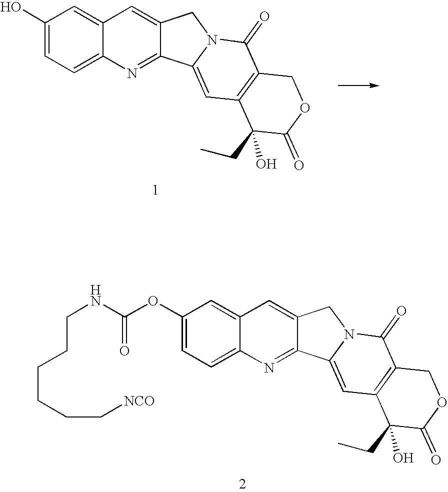

A 100-mL single-necked round-bottomed flask, is charged with a magnetic stirrer bar, 10-hydroxycamptothecin (600 mg, 1.65 mmol) and anhydrous DMF (40 mL). The flask is placed in an ultrasonic bath until all solid is dissolved. The mixture is stirred and 1,6-bis(isocyanate)hexane (2.77 mL, 16.5 mmol, 10 equivalent) is added, followed with triethylamine (2 mL). The flask is wrapped with alumni foil to protect from light. The reaction is monitored by TLC (dichloromethane/methanol, 95/5, V/V). The starting material has R_{f} 0.4, and the product R_{f} is 0.7. After 30 min, TLC confirms that the reaction is finished. The solvent is removed under vacuum till dry. The residue is then mixed with anhydrous ether (40 mL) and then the flask is placed in the ultrasonic bath for 30 s. The suspension is then kept for 3 h at 4° C. The solid is collected by suction filtration to yield the expected product (790 mg, 90%) as light yellow powder.

M.p. 230-235° C.

¹H NMR (DMSO-d₆, 200 MHz), δ=0.95 (t, J=7.32 Hz, 3H, CH₃), 1.40-1.70 (m, 8H, 4 CH₂), 1.90 (q, J=7.32 Hz, 2H, CH₂), 2.90 (t, J=7.20 Hz, 2H, CH₂), 3.20 (t, J=7.20 Hz, 2H, CH₂), 5.20 (s, 2H, CH₂), 5.40 (s, 2H, CH₂), 6.60 (s, 1H), 7.20 (s, 1H), 7.60 (m, 1H), 7.90 (s, 1H), 7.95 (m, 1H), 8.20 (d, 1H), 8.60 (s, 1H) ppm. (FIG. 16).

¹H NMR (DMSO-d, 500 MHz), δ=0.86 (t, J=7.3 Hz, 3H, CH₃), 1.30-1.60 (m, 8H, 4 CH₂), 1.90 (q, J=7.3 Hz, 2H, CH₂), 2.95 (t, J=7.2 Hz, 2H, CH₂), 3.20 (t, J=7.2 Hz, 2H, CH₂), 5.25 (d, J=8.5 Hz, 2H, CH₂), 5.40 (s, 2H, CH₂), 6.50 (d, J=2.46 Hz, 1H), 7.30 (d, J=5.7 Hz, 1H), 7.62 (m, 1H), 7.85 (m, 1H), 7.95 (m, 1H), 8.15 (m, 1H), 8.60 (d, J=7.2 Hz, 1H) ppm.

¹³C NMR (DMSO-d₆, 100 MHz), δ=7.76 (CH₃), 25.68, 26.01, 29.03, 29.17, 30.02, 30.47,42.51, 50.20, 65.24, 72.37,96.56, 118.59, 118.97, 126.24, 128.40, 130.20, 130.23, 130.98, 145.43, 145.51, 149.71, 149.99, 152.12, 154.03, 156.79, 172.43 ppm. (FIG. 17).

LSIMS (Matrix: Thioglycerol), m/e=641, 533, 429, 365, 321, 215. HRMS (LSIMS, Matrix: thioglyerol): found 533.20385, required 533.20363 for [C₂₈H₂₉N₄O₇]⁺. (FIG. 18).

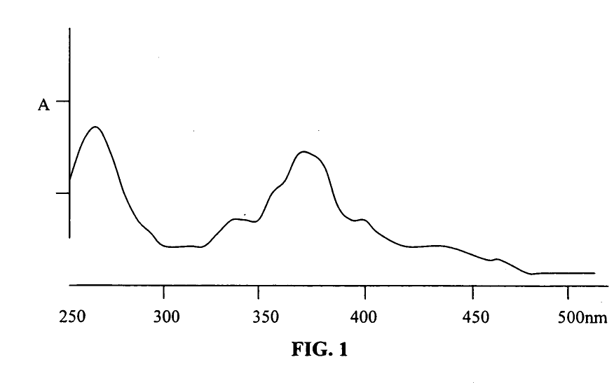

UV-vis (30% DMF in 10 mM, pH 7.4 PBS) λ(ε=382 (20 600), 280 (7 200), 259 (22000) nm. UV-vis (DMF) λ(ε)=382 (24 000), 280 (7 500), 259 (25 600). UV-Vis of 10-hydroxycamptothecin 6-isocyanatehexylcarbamate in (DMF) is shown in FIG. 1.

### 1,6(bis(10-hydroxycamptothecincarbamate)hexane [C₄₈H₄₄N₆O₁₂, FW=896]

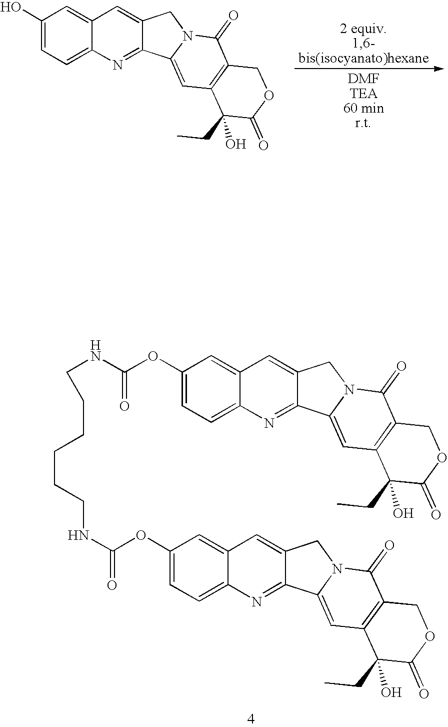

A 100-mL single-necked round-bottomed flask, is charged with a magnetic stirrer bar, 10-hydroxycamptothecin (200 mg, 0.55 mmol) and anhydrous DMF (10 mL). The flask is placed in an ultrasonic bath till all solid is dissolved. The mixture is stirred and 1,6-bis(isocyanate)hexane (0.181 mL, 1.10 mmol, 2.0 equivalent) is added, followed with triethylamine (0.5 mL). The flask is wrapped with alumni foil to protect from light. The reaction is monitored by TLC (dichloromethane/methanol, 95/5, V/V). The starting material has R_{f} 0.4, and the product R_{f} is 0.9. After 30 min, TLC confirms that the reaction is finished. The solvent is removed under vacuum till dryness. The residue is then mixed with anhydrous ether (40 mL) and then the flask is placed in the ultrasonic bath for 30 s. The suspension is then kept for 3 h at 4° C. The solid is collected by suction filtration to yield the expected product (230 mg, 93%) as light yellow powder.

M.p.>280° C.

¹H NMR (DMSO-d₆, 200 MHz), δ=0.95 (t, J=7.32 Hz, 6H, 2 CH₃), 1.40-1.70 (m, 8H, 4CH₂), 1.90 (q, J=7.32 Hz, 4H, 2CH₂), 2.95 (t, J=7.20 Hz, 2H, CH₂), 3.20 (t, J=7.20 Hz, 2H, CH₂), 5.23 (s, 4H, 2 CH₂), 5.40 (s, 4H, 2 CH₂), 6.60 (s, 2H), 7.20 (s, 2H), 7.60 (m, 2H), 7.90 (s, 2H), 7.95 (m, 2H), 8.20 (d, 2H), 8.60 (s, 2H) ppm. (FIG. 19).

¹H NMR (DMSO-d₆, 500 MHz), δ=0.88 (t, J=7.3 Hz, 6H, 2 CH₃), 1.30-1.60 (m, 8H, 4 CH₂), 1.85 (q, J=7.3 Hz, 4H, 2 CH₂), 2.95 (t, J=7.2 Hz, 2H, CH₂), 3.10 (t, J=7.2 Hz, 2H, CH₂), 5.25 (d, J=8.5 Hz, 4H, 2 CH₂), 5.45 (s, 4H, 2 CH₂), 6.50 (s, 2H), 7.30 (d, J=5.7 Hz, 2H), 7.62 (m, 2H), 7.85 (m, 2H), 7.95 (m, 2H), 8.15 (m, 2H), 8.60 (d, J=7.2 Hz, 2H) ppm.

¹³C NMR (DMSO-d₆, 100 MHz), 67 =7.72, 25.98, 29.10, 30.00, 30.30, 50.14, 65.21, 72.33, 96.52, 118.53, 118.93, 126.18, 128.35, 130.06, 130.91, 145.38, 146.38, 149.69, 149.95, 152.04, 153.67, 156.74, 158.09, 172.38 ppm. (FIG. 20).

LSIMS (Matrix: thioglyerol), m/e=1039, 897, 809, 533, 365, 321. (FIG. 21).

HPLC (30% acetonitrile in water), Tr=18.2 min.

Synthesis of 10CPT-CHU-P97 conjugate 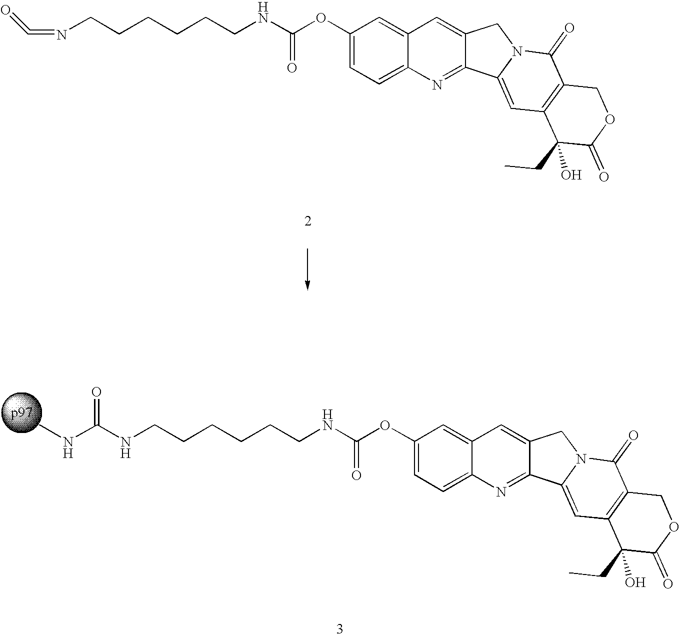
P97 (lot# 18, OD at 280 nm 1.67, c=1.37 mg/mL, 108 mL, 148 mg, 1.5682×10⁻³ mM) is placed in a 250 mL flask. To this solution, a solution of 10-hydroxycamptothecin 6-isocyanatehexylcarbamate (2, 83.4 mg, 0.1568 mmol, 100 equiv. in DMF 50 mL. DMF is 30.7% for the whole solution). The mixture is stirred at room temperature for 16 hr, then purified by dialysis using snake skin tube (MWCO=10K) against PBS (10 mM, pH=7.4) to yield pure conjugate 190 mL. Protein p97 recovery is 96%.

SYN-026 (10CPT-CAH-p97) (Method 1)

| | | | 10-OH-CPT | | |
|---|---|---|---|---|---|
| Sample | 280 nm | 382 nm | (mg/mL) | p97 (mg/mL) | MSR |
| SYN026 | 1.2634 | 0.9526 | 0.017807 | 0.748157 | 6.17 |

Determination of MSR

Theory: 10-hydroxycamptothecin has a strong absorption at 382 nm with molar extinction coefficient 25500 (in DMF, see literature Chem. Pharm. Bull. 1991, 39 (12), 3183-3188.), while there is absorption for p97 at the same wavelength. Therefore, one can determine the 10-hydroxycamptothecin concentration by measuring the UV-Vis absorption at 382 nm. P97 has strong absorption at 280 nm, while 10-hydroxycamptothecin has weak absorption at the same wavelength. Thus the concentration of p97 could be deduced from the result of that the total absorbance at 280 nm minus the absorbance of 10-hydroxycamptothecin at the same wavelength.

10-hydroxycamptothecin Standard UV-Vis:

(1). Preparation of stock solution: 2.15 mg 10-hydroxycampothecin is dissolved in 5 mL DMF.

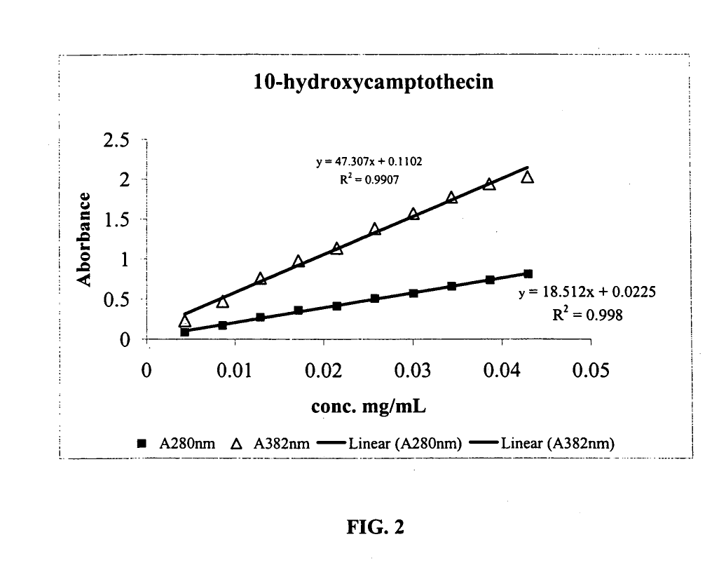

(2). Preparation of samples: aliquots (mLs) of stock solution is taken and mixed with required amount of DMF to have a total volume 0.30 mL. Then 0.70 mL PBS buffer (10 mmol, pH 7.4) is added and mixed with the 0.3 mL DMF. All samples are recorded their UV-Vis absorbance at 280 nm and 382 nm. The results are listed in Table 1 and FIG. 2.

TABLE 1
UV-Vis of 10-hydroxycamptothecin in 30% DMF-PBS (pH 7.4)

| conc. mg/mL | A280 nm | A382 nm | ε (280 nm) | ε (382 nm) |
|---|---|---|---|---|
| 0.0043 | 0.0886 | 0.2309 | 7500 | 19550 |
| 0.0086 | 0.1739 | 0.4736 | 7360 | 20045 |
| 0.0129 | 0.2733 | 0.7631 | 7712 | 21532 |
| 0.0172 | 0.3612 | 0.9811 | 7644 | 20760 |
| 0.0215 | 0.4135 | 1.1376 | 7000 | 19260 |
| 0.0258 | 0.5086 | 1.3844 | 7175 | 19532 |
| 0.0301 | 0.5711 | 1.5710 | 6906 | 18998. |
| 0.0344 | 0.6622 | 1.7752 | 7007 | 18784 |
| 0.0387 | 0.7383 | 1.9412 | 6944 | 18258 |
| 0.043 | 0.8124 | 2.0325 | 6877 | 17205 |
| average | | | 7210 | 19400 |

To measure the MSR, one just simply measures the absorbance of the conjugate at 382 and 280 nm. Then using the following equation to calculate the concentrations of p97, 10-hydroxycamptothecin as well as the MSR:

Method 1: Using the Standard Curve

Concentration of 10-hydroxycamptotheicn:

$$\begin{matrix}{C}_{10 CPT} ( mg \text{/} mL ) = 0.02114 * {A}_{382} \text{-} 0.002329 \\ {C}_{10 CPT} ( mol \text{/} L ) = ( 5.8072 * {A}_{382} \text{-} 0.64 ) * {10}^{- 5}\end{matrix}$$

Concentration of p97

$$\begin{matrix}{A}_{280} ( p 97 ) = {A}_{280} ( conjugate ) - {A}_{280} ( 10 CPT ) \\ = {A}_{280} - ( 18.512 ( 0.02114 * {A}_{382} - 0.002329 ) + 0.0225 ) \\ = {A}_{280} - 0.3913 * {A}_{382} + 0.02061\end{matrix}$$

$${C}_{p 97} ( mg \text{/} mL ) = ( {A}_{280} - 0.3913 * {A}_{382} + 0.02061 ) / 1.218$$

MSR Calculator:

$$MSR = ( {C}_{10 CPT} / {C}_{p 97} ) * ( 94420 / 364 )$$

Method 2: Using the Lambert-Beer Law.

The molar extinction coefficients for 10-hydroxycamptothecin at 382 and 280 nm are 19400 and 7210 mol⁻¹*L*cm⁻¹ respectively.
A=ε1c

A is absorbance; ε is molar extinction coefficient in mol⁻¹*L*cm⁻¹; c is concentration in mol*L⁻¹.

$$\begin{matrix}\begin{matrix} {C}_{10 CPT} = ( {A}_{382} / 19400 ) * 364 = 0.01876 * {A}_{382} mg \text{/} mL \\ {C}_{p 97} = ( {A}_{280} - {A}_{382} * ( 7210 / 19400 ) ) / 1.218 = ( {A}_{280} - 0.3716 * {A}_{382} ) / 1.218 mg \text{/} L\end{matrix} \\ MSR = ( {C}_{10 CPT} / {C}_{p 97} ) * ( 94420 / 364 )\end{matrix}$$

Test of Method 1 Using Known Concentrations of 10-hydroxycamptothecin and p97 Mixture

TABLE 2
test of the calculator (method 1)

| | | | 10-OH- | | | |
|---|---|---|---|---|---|---|
| Abs | | | CPT | P97 | MSR | |
| Sample | 280 nm | 382 nm | (mg/mL) | (mg/mL) | found | MSR theory |
| A | 1.3704 | 0.5499 | 0.009295 | 0.965384 | 2.50 | 2.3 |
| B | 1.4133 | 0.8082 | 0.014755 | 0.917620 | 4.17 | 3.5 |
| ½B | 0.6689 | 0.381 | 0.005724 | 0.443704 | 3.35 | 3.5 |
| C | 1.5299 | 1.3466 | 0.026136 | 0.840375 | 8.07 | 6.34 |
| D | 1.5686 | 1.5778 | 0.031023 | 0.797869 | 10.09 | 7.0 |
| E | 1.6151 | 1.7929 | 0.035570 | 0.766939 | 12.03 | 8.14 |

Note:
the mixtures are prepared as the following: stock 10-hydroxycamptothecin in DMF is taken to make 0.15 mL DMF, then mixed with 0.35 mL known concentration of p97. The mixed samples then are recorded their UV-Vis at 382 nm and 280 nm.

TABLE 3
Test of the calculator (method 2)

| | | | 10-OH- | | | |
|---|---|---|---|---|---|---|
| Abs | | | CPT | P97 | MSR | |
| Sample | 280 nm | 382 nm | (mg/mL) | (mg/mL) | found | MSR Theory |
| A | 1.3704 | 0.5499 | 0.010318 | 0.957332 | 2.80 | 2.3 |
| B | 1.4133 | 0.8082 | 0.015164 | 0.913738 | 4.30 | 3.5 |
| ½B | 0.6689 | 0.381 | 0.007149 | 0.432924 | 4.28 | 3.5 |
| C | 1.5299 | 1.3466 | 0.025266 | 0.845186 | 7.75 | 6.34 |
| D | 1.5686 | 1.5778 | 0.029604 | 0.806413 | 9.52 | 7 |
| E | 1.6151 | 1.7929 | 0.033640 | 0.778957 | 11.20 | 8.14 |

Note:
the mixtures are prepared as the following: stock 10-hydroxycamptothecin in DMF is taken to make 0.15 mL DMF, then mixed with 0.35 mL known concentration of p97. The mixed samples then are recorded their UV-Vis at 382 nm and 280 nm.

From the results of Table 2 and Table 3, it could be deduced that Method 1 is good at low concentrations, at which the MSR results are in good agreement with the theory values, and method 2 is good for high concentration mixtures.

### Example 5

### Exemplary Ssynthesis of Isocyanate Linkers by Method A Synthesis of mono (tert-butyl acrylate) polyethyleneglycol

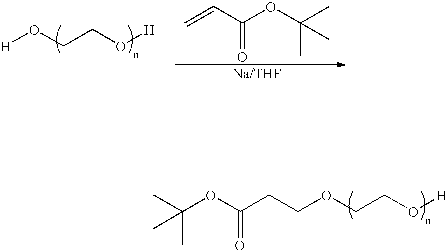

Poly(ethylene glycol) (1.0 mol) was dissolved in THF. The mixture is stirred. Sodium (0.212 g, 9.2 mmol) was added. The mixture was stirred at 40° C. until all the sodium was dissolved using a water bath. Then the water bath was removed, and tert-butylacrylate (51 mL, 0.34 mmol). The mixture was stirred at room temperature for overnight. 2% HCl solution was added slowly under stirring to adjust pH 7.0. Solvent is removed under vacuum. The light yellow oily residue was taken up by ethyl acetate (500 mL) and partitioned with brine (100 mL). The organic layer was separated and the aqueous layer was extracted by ethyl acetate (6×150 mL). The organic layer and extracts were combined and washed with brine (2×200 mL), and dried over anhydrous sodium sulfate. The solvent is removed under vacuum to afford yellow oily crude product, which was dried under high vacuum overnight to give the expected product, which was directed used without further purification. Analytic sample is purified silica gel chromatographic column using dichloromethane-methanol (95/5, v/v) as eluent.

### Synthesis of Tert-Butyl 3-(tetraethylene glycol)propanoate [C₁₅H₃₀O₇, FW=322]

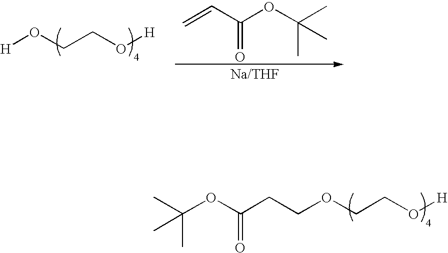

Prepared according Method A, yield 84%

IR, ν=3450, 2870, 1726, 1451, 1365, 1329, 1249, 1105, 1068, 945, 887, 847, 756, 528 cm⁻¹. ¹H NMR (200 MHz, CDCl₃), δ=1.50 (s, 9H, 3 CH₃), 2.50 (t, J=7.0 Hz, 2H, CH₂), 3.80 (m, 19H, 9 OCH₂, OH) ppm. ¹³C NMR (50 MHz, CDCl₃), δ=27.50 (CH₃), 27.85 (CH₂), 36.00 (CH₂), 62.34, 66.61, 70.28 (large), 72.30, 76.36, 80.18 (OCMe₃), 170.62 (COO) ppm LSIMS (matrix: thioglyerol), m/e=333 [M+1]⁺. Anal. Calcd. for C₁₅H₃₀O₇.H₂O, C, 52.92; H, 9.48. Found: C, 52.75; H, 9.32.

Reference: Seitz, O.; Kunz, H. J. Org. Chem. 1997, 62, 813-826.

### Synthesis of Tert-Butyl 3-[tri(ethylene glycol)]propanoate [C₁₃H₂₆O₆, FW=278]

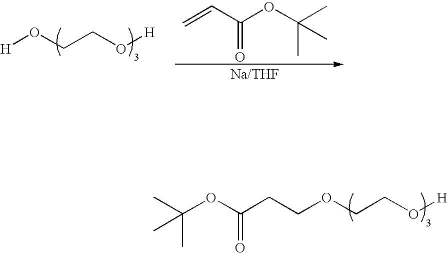

Prepared according to Method A, yield 80.5%

IR, ν=3340, 2869, 1726, 1456, 1392, 1365, 1330, 1252, 1111, 1064, 943, 887, 756, 547 cm-1. ¹H NMR (200 MHz, CDCl₃), δ=1.53 (s, 9H, 3 CH3), 2.50 (t, J=7.0 Hz, 2H, CH2), 3.85 (m, 14H, 7 OCH2, OH) ppm. ¹³C NMR (50 MHz, CDCl₃), δ=28.5 (CH3), 35.2 (CH2), 62.1, 64.5, 70.3 (large), 72.15, 82.2 (OCMe3), 172.3 (COO) ppm MS (ES), m/e=296 [M+Na]+. Anal. Calcd. for C13H₂₆O6, C, 56.10; H, 9.42. Found: C, 56.45; H, 9.32.

Reference: Seitz, O.; Kunz, H. J. Org. Chem. 1997, 62, 813-826.

### Synthesis of Tert-Butyl 3-[di(ethylene glycol)]propanoate [C₁₁H₂₂O₅, FW=234]

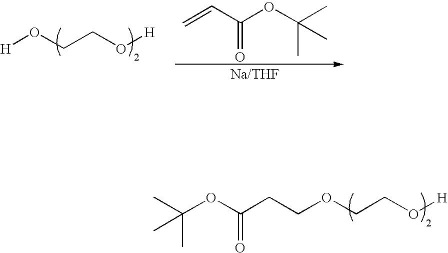 Prepared according Method A, yield 72%

IR, ν=3452, 2872, 1726, 1454, 1392, 1367, 1332, 1252, 1157, 1112, 1062, 933, 889, 846, 754, 548, 517 cm-1. ¹H NMR (200 MHz, CDCl₃), δ=1.20 (s, 9H, 3 CH3), 2.20 (t, J=7.0 Hz, 2H, CH2), 3.40 (m, 6H, 3 OCH2) ppm. 13C NMR (50 MHz, CDCl3), δ=27.9, 36.0, 61.37, 66.6, 70.1 (3 carbons), 72.3, 80.3, 171.1 (COO) ppm MS (ES), m/e=257 [M+Na]+. Anal. Calcd. for C11H22O5, C, 56.39; H, 9.46. Found: C, 56.78; H, 9.65.

Reference: Seitz, O.; Kunz, H. J. Org. Chem. 1997, 62, 813-826.

### Example 6

### Method B—General procedure for the synthesis of mono(butyl acrylate) polyethyleneglycol

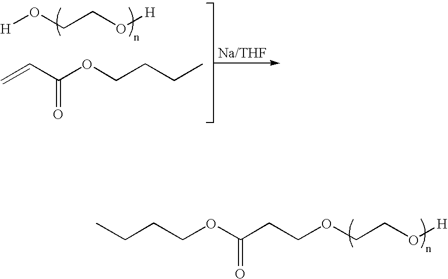

poly(ethylene glycol) (1.0 mol) was dissolved in THF. The mixture is stirred. Sodium (0.212 g, 9.2 mmol) was added. The mixture was stirred at 40° C. till all sodium dissolved using a water bath. Then the water bath was removed, and butyl acrylate (51 mL, 0.34 mmol). The mixture was stirred at room temperature for overnight. 2% HCl solution was added slowly under stirring to adjust pH 7.0. Solvent is removed under vacuum. The light yellow oily residue was taken up by ethyl acetate (500 mL) and partitioned with brine (100 mL). The organic layer was separated and the aqueous layer was extracted by ethyl acetate (6×150 mL). The organic layer and extracts were combined and washed with brine (2×200 mL), and dried over anhydrous sodium sulfate. The solvent is removed under vacuum to afford yellow oily crude product, which was dried under high vacuum overnight to give the expected product, which was directed used without further purification. Analytic sample is purified silica gel chromatographic column using dichloromethane-methanol (95/5, v/v) as eluent.

### Tert-butyl-3-(tri(ethylene glycol)propanoate [C₁₁H₂₂O₅, FW=234]

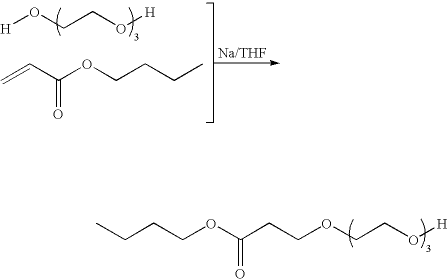 Prepared according to method B. Yield 84%.

IR, ν=3448, 2872, 1732, 1458, 1352, 1253, 1182, 1112, 937, 885, 812,532 cm-1. ¹H NMR (200 MHz, CDCl₃), δ=1.10 (t, J=7.0 Hz, 3H, CH3), 1.40 (m, 2H, CH2), 1.60 (m, 2H, CH2), 2.30 (t, J=7.0 Hz, 2H, CH2), 3.00 (Br s, 1H, OH), 3.65 (m, 10H, 5 OCH2), 4.00 (m, 2H, OCH2) ppm. ¹³C NMR (50 MHz, CDCl₃), δ=13.8, 18.9,30.5,34.9,61.5, 64.3, 66.5, 70.3 (4 carbons), 72.5, 171.2 (COO) ppm MS (ES), m/e=257 [M+Na]+. Anal. Calcd. for C1122O5, C, 56.39; H, 9.46. Found: C, 56.68; H, 9.30.

Reference: Seitz, O.; Kunz, H. J. Org. Chem. 1997, 62, 813-826.

### Tert-butyl-3-[dodeca(ethylene glycol)]propanoate [C₃₁H₆₂O₁₅, FW=674]

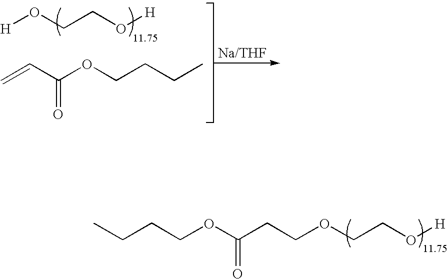 Prepared according to method B using PEG 300 as one of the starting material. Yield 68%.

IR, ν=3448, 2868, 2361, 1732, 1685, 1458, 1350, 1249, 1184, 1101, 943, 842, 812, 542 cm-1. ¹H NMR (200 MHz, CDCl₃), δ=1.05 (t, J=7.0 Hz, 3H, CH3), 1.40 (m, 2H, CH2), 1.60 (m, 2H, CH2), 2.40 (t, J=7.0 Hz, 2H, CH₂), 3.00 (Br s, 1H, OH), 3.65 (m, 50H, 25 OCH2), 4.00 (m, 2H, OCH2) ppm. ¹³C NMR (50 MHz, CDCl3), δ=13.6, 18.9, 30.5, 35.1, 61.52, 63.5. 66.2, 70.5 (large peak), 72.5, 172.4 (COOH) ppm.

Reference: Seitz, O.; Kunz, H. J. Org. Chem. 1997, 62, 813-826.

### Isocyanato tert-butyl acrylate linker 1 [C₂₃H₄₂N₂O₉, FW=490]

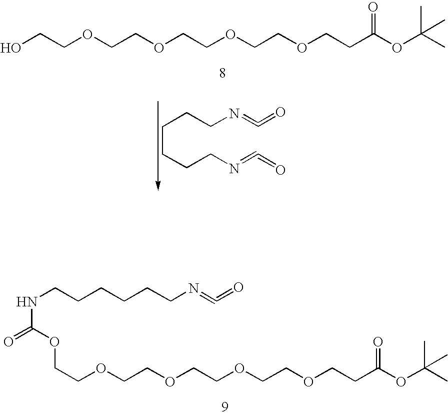

1,6-Diisocyanatohexane (5 mL, 0.03 mol) is dissolved in anhydrous dichloromethane (50 mL). Then a solution of tert-butyl tetra(ethylene glycol)propionate (10.28 g, 95%, 0.03 mol) in dichloromethane (50 mL) and triethylamine (1.5 mL) is added dropwise under stirring over a period of 30 min. The mixture is stirred at room temperature overnight. Solvent is removed under vacuum. The oily residue is pumped under high vacuum overnight to give the crude product 12.0 g (82%), which is directly used without further purification. Analytic sample is purified silica gel chromatographic column using dichloromethane as eluent.

^{[207]}IR, ν=3337, 2931, 2866, 2268, 1720, 1627, 1535, 1458, 1365, 1247, 1107, 949, 846, 775, 756 cm⁻¹

¹H NMR (200 MHz, CDCl₃), δ=1.60 (s, 9H, 3 CH₃), 1.65 (m, 8H, 4 CH₂), 2.45 (t, J=7.0 Hz, 2H, CH₂), 3.20(m, 2H, CH₂), 3.32 (m, 2H, CH₂), 3.60 (m, 14H, 7 OCH₂), 4.20 (t, J=6.8 Hz, 2H, CH₂), 5.10 (s, 1H, NH) ppm

¹³C NMR (50 MHz, CDCl₃), δ=25.70 (CH₂), 27.72(CH₂), 29.77 (CH₃), 31.06 (CH₂), 36.20(CH₂), 40.76 (CH₂), 42.78 (CH₂), 45.43 (CH₂), 63.74 (OCH₂), 66.80 (OCH₂), 70.51 (br, OCH₂), 80.67 (OCMe₃), 122.33 (N═C═O), 156.41 (NH—CO—O), 170.80 (COO) ppm. LSIMS (matrix: thioglyerol), m/e=491 [M+1]⁺.

Anal. Calcd. for C₂₃H₄₂N₂O₉.0.5H₂O, C, 55.29; H, 8.68; N, 5.61. Found: C, 54.88; H, 8.45; N, 5.20.

### Example 7

### Synthesis of SYN027 Bioconjugate

SYN-027: 10-hydroxy-camptothecin is covalently bound to p97 through a CHC-PEG4 linker. For easy remember, we can call this conjugate for 10CPT-CHC-PEG4-p97. [C—carbamate; H—hexyl; PEG4—four ethylene glycol units].
Chemical Structure: 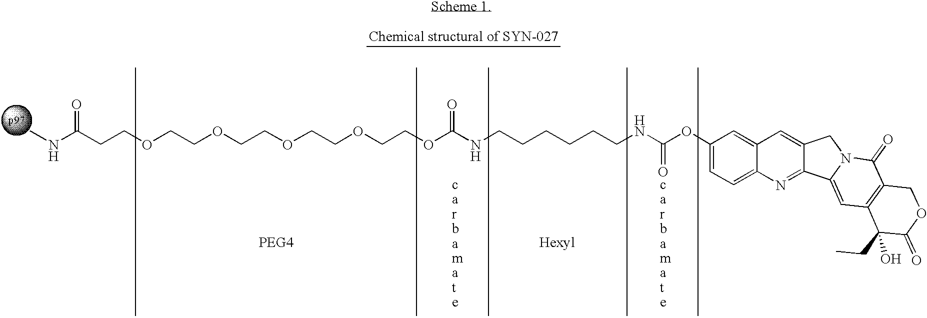
Synthesis Flowchart:

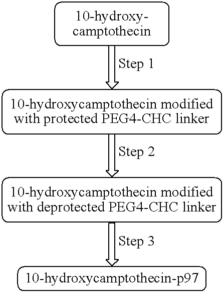

The total synthesis of SYN-027 conjugate includes the following three steps (Scheme 2): step 1: treating 10-hydroxycamptothecin 2 with isocyanato-tert-butyl ester linker 1 to generate the expected intermediate—tert-butyl ester CHC-PEG4-10-hydroxycamptothecin 3;

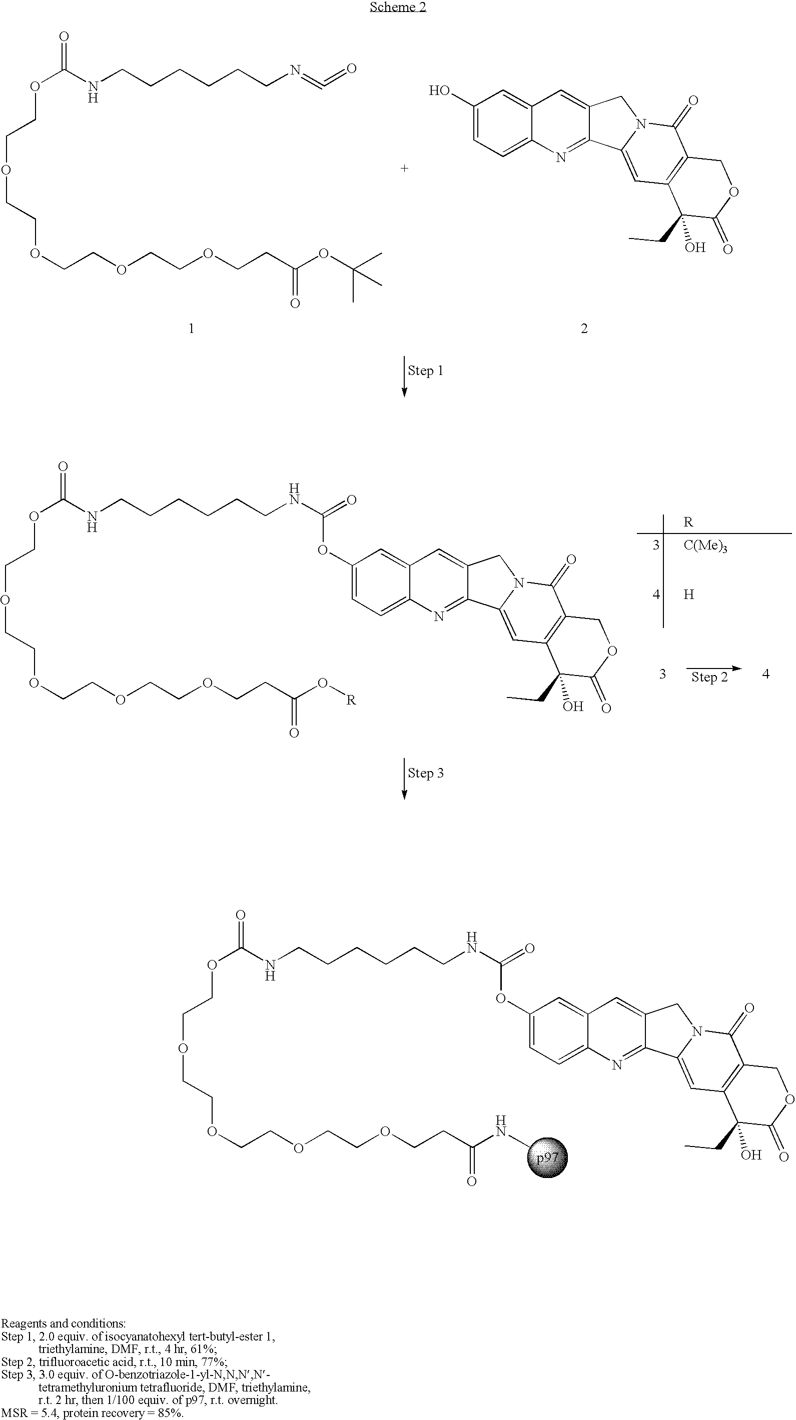

Step 2: treating this intermediate with trifluoroacetic acid to offer the deprotect CHC-PEG4-10-hydroxycamptothecin 4; step 3: activating the free acid using O-benzotrizole-1-yl-N,N,N′,N′-tetramethyluronium borontetrafluoride (BTTU, then coupling with p97 directly to yield the expected conjugate.

Optimization of the Synthesis of Conjugate (SYN027).

Too much high MSR of 10-hydroxycamptothecin bind to p97 may be effect the transcytosis of p97. And too low MSR will reduce the efficiency to delivery the drug. Therefore, we wish to synthesize the conjugate with MSR of 10-hydroxycamptothecin 4-6. To find the best reaction condition, we fix the reaction to run at room temperature, DMF 30% to investigate the effect of reaction time and different molar excess of 10-hydroxycamptothecin used. The reaction is monitored by FPLC. Exemplary FPLC profiles of 10-hydroxycamptothecin-p97 conjugate at various reaction time are shown FIGS. 3-8.

FPLC clearly shows that almost 95% of p97 is converted to the conjugate after 4 hour reaction time (FIG. 3˜5). The ratio of 10-hydroxycamptothecin bound to p97 could be also easily monitored by the peak ratio of the conjugate at 382 and 280 nm (Table 2).

TABLE 2
changes of peak ratio of the conjugate at 382 and 280 nm
at different time*

| | Area-of-peak @ 280 nm | Heigh-of-peak @ 280 nm |
|---|---|---|
| Reaction time (h) | Area-of-peak @ 338 nm | Heigh-of-peak @ 382 nm |
| 1 | 2.89 | 2.67 |
| 2 | 2.47 | 2.62 |
| 4 | 2.47 | 2.38 |
| 6 | 2.09 | 2.11 |
| 9 | 2.06 | 2.08 |
| 20 | 1.41 | 1.45 |
| 28 | 1.42 | 1.44 |

*Reaction condition: DMF = 30%, compound 4 50 equivalent, r.t., pH = 7.40. FPLC: Column: BioSep 300 size exclusive, buffer: 0.1M sodium phosphate, pH 6.8, flow rate: 1 mL/min.

From Table 2 (or FIG. 9), the MSR reach the highest after 20 h reaction. This suggests that the reaction is finished after 20 hr.

FIGS. 10-13 shows the effect of different molar equivalent excess to the MSR. Reaction conditions are the following: DMF=30%, reaction time 3 hours at room temperature, p97 concentration 1.23 mg/mL, pH=7.40. Column: Biosep 300 size exclusive, eluent buffer: 0.1 M sodium phosphate monobasic, pH 6.8, flow rate: 1.0 ml/mL.

The linker 1 is synthesized as in the following (scheme 3).

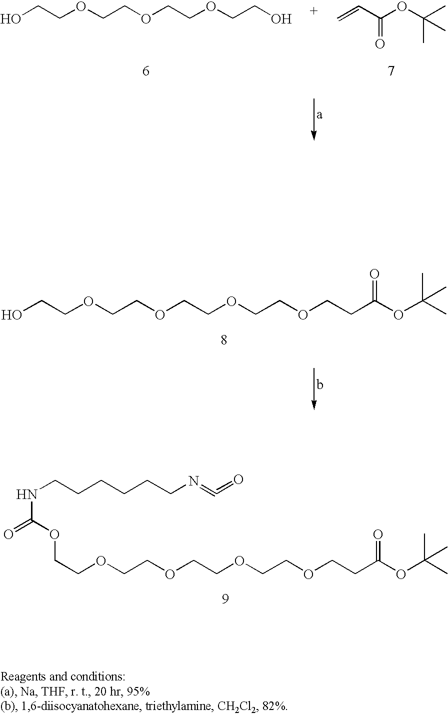

Treating tetra(ethylene glycol) 6 with one equivalent of tert-butyl acrylate and a catalytic amount of sodium in anhydrous tetrahydrofurane overnight at room temperature, a mono tert-butyl acrylate linked tetra(ethylene glycol) 8 is obtained in 95%, which further reacts with 1,6-diisocyanatohexane in dichloromethane and triethylamine to offer a isocyanato-tert-butyl acrylate linker 9 in 82%.

Brief summary for this new conjugate
One step synthesis to p97.
Carbamate bond to the 10-hydroxycamptothecin, which may be biodegradable and be able to release free 10-hydroxycamptothecin after the drug is delivered to the target sites.
Containing 4 PEG units, which will increase the drug water solubility.

High yield and very efficient synthesis for both the modified 10-hydroxycamptothecin and the p97 conjugate.
Confirmed new isocyanato linkage technologies.

For experimental procedures for the synthesis of this conjugate see the experimental section.

### Determination of MSR

Theory: 10-hydroxycamptothecin has a strong absorption at 382 nm with molar extinction coefficient 25500 (in DMF, see literature Chem. Pharm. Bull. 1991, 39 (12), 3183-3188.), while there is absorption for p97 at the same wavelength. Therefore, one can determine the 10-hydroxycamptothecin concentration by measuring the UV-Vis absorption at 382 nm. P97 has strong absorption at 280 nm, while 10-hydroxycamptothecin has weak absorption at the same wavelength. Thus the concentration of p97 could be deduced from the result of that the total absorbance at 280 nm minus the absorbance of 10-hydroxycamptothecin at the same wavelength.
Method 1: Using the Standard Curve

$$\begin{matrix}\begin{matrix} {C}_{10 CPT} ( mg \text{/} mL ) = 0.02114 * {A}_{382} - 0.002329 \\ {C}_{p 97} ( mg \text{/} mL ) = ( {A}_{280} - 0.3913 * {A}_{382} + 0.02061 ) / 1.218\end{matrix} \\ MSR = ( {C}_{10 CPT} / {C}_{p 97} ) * ( 94420 / 364 )\end{matrix}$$

Method 2: Using the Lambert-Beer Law.

The molar extinction coefficients for 10-hydroxycamptothecin in 30% DMF-PBS (20 mmol, pH 7.5) at 382 and 280 nm are 19400 and 7210 mol⁻¹*L*cm⁻¹ respectively.

$$\begin{matrix}\begin{matrix} {C}_{10 CPT} ( mg \text{/} mL ) = 0.01876 * {A}_{382} \\ {C}_{p 97} ( mg \text{/} mL ) = ( {A}_{280} - 0.3716 * {A}_{382} ) / 1.218\end{matrix} \\ MSR = ( {C}_{10 CPT} / {C}_{p 97} ) * ( 94420 / 364 )\end{matrix}$$

SYN-027 (10CPT-PEG4-p97) Method 1

| | | | 10-OH-CPT | | |
|---|---|---|---|---|---|
| Sample | 280 nm | 382 nm | (mg/mL) | P97 (mg/mL) | MSR |
| SYN027 | 1.2222 | 0.8425 | 0.015481 | 0.749631 | 5.36 |

SYN-027 (10CPT-PEG4-p97) Method 2

| Abs | | | 10-OH-CPT | | |
|---|---|---|---|---|---|
| Sample | 280 nm | 382 nm | (mg/mL) | P97 (mg/mL) | |
| A | 1.2222 | 0.8425 | 0.015808 | 0.746375 | 5.49 |

### Isocyanatohexyl-carbamate—PEG4-t-butyl ester linked 10-Hydroxycamptothecin [C₄₃H₅₈N₄O₁₄, FW=854]

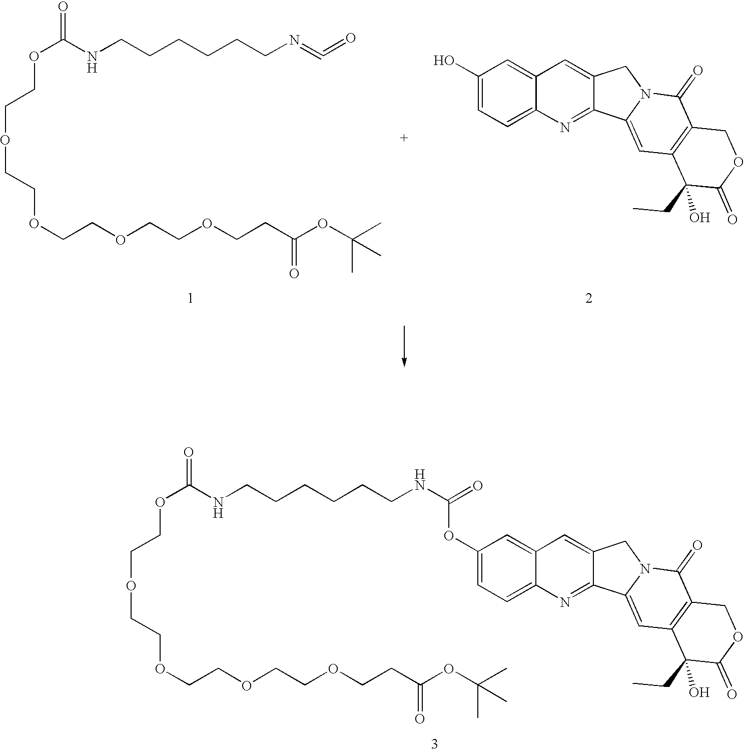

A 100-mL single-necked round-bottomed flask equipped with a magnetic stirrer bar, is charged with 10-hydroxycamptothecin (500 mg, 1.37 mmol) and anhydrous DMF (30 mL) and triethylamine (1.0 mL). The flask is placed in an ultrasonic bath till all solid is dissolved. The mixture is stirred. A solution of isocyanatohexyl-carbamato-PEG4-tert-butylester linker (1, 1.35 g, 2.75 mmol, 2.0 equivalent) in dichloromethane (10 mL) is added. The flask is wrapped with alumni foil to protect from light. The reaction is monitored by TLC (dichloromethane/methanol, 95/5, V/V). The starting material has R_{f} 0.4, and the product R_{f} is 0.6. After 2 hr, TLC confirms that the reaction is finished. The solvent is removed under vacuum till dryness. The residue is taken up by methanol (5 mL), then mixed with anhydrous ether (40 mL). The resulted suspension mixture is placed in the ultrasonic bath for 30 s, and then kept for 3 h at 4° C. The solid is collected by suction filtration to yield the expected product (717 mg, 61%) as light yellow powder.

M.p. 180-183° C.

IR, ν=3313,2929, 2860, 1718, 1654, 1600, 1541, 1489, 1446, 1348, 1227, 1195, 1103, 1043, 1001, 916, 835, 800, 960 cm-1. (FIG. 26).

¹H NMR (DMSO-d₆, 200 MHz), δ=0.98 (t, J=7.2 Hz, 3H, CH₃), 1.20-1.40 (m, 17H, 4 CH₂, 3CH₃), 1.85 (q, J=7.2 Hz, 2H, CH₂), 3.06 (t, J=6.9 Hz, 2H, CH₂), 3.12 (t, J=6.9 Hz, 2H, CH₂), 3.41 (m, 14H, 7 OCH₂), 4.04 (m, 2H, OCH₂), 5.24 (s, 2H, CH₂), 5.43 (s, 2H, CH₂), 6.52 (s, 1H), 7.26 (m, 1H), 7.61 (m, 1H), 7.98 (m, 1H), 8.08 (m, 1H), 8.11 (m, 1H), 8.68 (m, 1H) ppm. (FIG. 23).

¹H NMR (DMSO-d₆, 400 MHz), δ=0.85 (t, J=7.2 Hz, 3H, CH₃), 1.20 (m, 2H, CH₂), 1.30 (m, 4H, 2 CH₂), 1.40 (s, 9H, 3 CH₃), 1.50 (m, 2H, CH₂), 1.80 (t, J=7.2 Hz, 2H, CH₂), 2.41 (t, J=6.2 Hz, 2H, CH₂), 2.90 (m, 2H, CH₂), 3.10 (m, 2H, CH₂), 3.35 (obscured with water peak, m, 2H, CH₂) 3.55 (m, 14H, 7 OCH₂), 4.05 (m, 2H, OCH₂), 5.25 (m, 2H, CH₂), 5.45 (s, 2H, CH₂), 6.50 (s, 1H), 7.20 (m, 1H), 7.38 (m, 1H), 7.65 (m, 1H), 7.85 (m, 1H), 7.95 (m, 1H), 8.15 (m, 1H), 8.62 (m, 1H),

¹³C NMR (DMSO-d₆, 100 MHz), δ=7.75 (CH₃), 25.94, 27.55 (CH₃), 29.13, 29.34, 30.01, 30.31, 35.82, 50.18, 62.96, 65.34, 66.20, 68.89, 69.68 (m, 9 OCH₂), 72.36, 79.69, 96.56 (CH), 118.57 (CH), 118.97, 126.23 (CH), 128.40, 130.10 (CH), 130.20, 130.97 (CH), 145.43, 145.50, 149.72, 150.00, 152.10, 154.01, 156.14, 156.78, 170.38 (COO), 172.43 (COO) ppm. (FIG. 24).

LSIMS (Matrix: Thioglycerol), m/e=855 [M⁺+1], 799, 365. HRMS (LSIMS, Matrix: thioglyerol): found 855.40251, required 855.40278 for [C₄₃H₅₉N₄O₁₄]⁺. (FIG. 25). UV-vis (DMF) λ(ε)=295 (11 400), 332 (13 400), 368 (32 600), 382 (28 400) nm. UV-vis (methanol) λ(ε)=292 (9 350), 330 (14 400), 362 (29 300), 375 (28 200) nm. UV-vis (DMSO) λ(ε)=286 (27 600), 332 (11 200), 368 (27 9000, 385 (25 000) nm. (FIG. 27).

Anal. Calcd. for C₄₃H₅₈N₄O₁₄.0.5H₂O: C, 59.78; H, 6.88; N, 6.49. Found: C, 60.04; H, 7.04; N, 6.11.

### Isocyanatohexyl-carbamate—PEG4-acid linked 10-Hydroxycamptothecin [C₃₉H₅₀N₄O₁₄, FW 798]

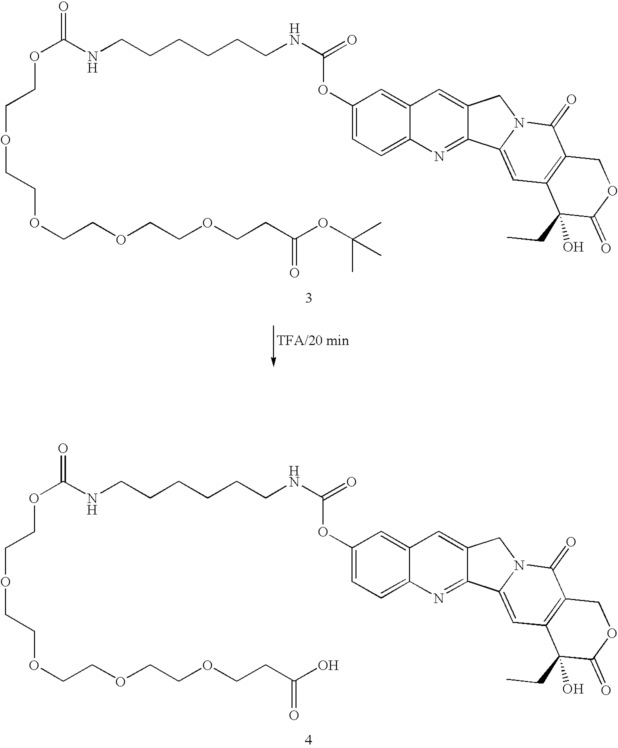

tert-Butyl-PEG4-carbamato-hexyl-carbamato-10-hydroxycamptothecin (3, 500 mg, 0.585 mmol) is placed in a 50 mL round-bottomed flask equipped with a magnetic stirrer. Trifluoroacetic acid (10 mL) is added. The mixture is stirred at room temperature for 20 min. Then anhydrous ether (60 mL) is added slowly over a period of 5 min. The suspension is then placed over an ultrasonic bath for 2 min. The yellow solid is collected by suction filtration. The crude product is then re-dissolved in a minimum amount of DMF, and precipitated by anhydrous ether. The expected compound 4 is obtained (429 mg, 92%) after filtration and drying under vacuum overnight.

M.p. 195-199° C. (dec.)

IR, ν=3311, 2920, 2858, 1730, 1655, 1600, 1539, 1498, 1443, 1348, 1226, 1195, 1151, 1103, 1043, 1001, 914, 833 cm-1. (FIG. 31)

¹H NMR (DMSO-d₆, 200 MHz), δ=0.95 (t, J=7.2 Hz, 3H, CH₃), 1.25 (m, 8H, 4 CH₂), 1.85 (q, J=7.2 Hz, 2H, CH₂), 3.05 (t, J=6.9 Hz, 2H, CH₂), 3.10 (t, J=6.9 Hz, 2H, CH₂), 3.40 (m, 14H, 7 OCH₂), 4.00 (m, 2H, OCH₂), 5.20 (s, 2H, CH₂), 5.40 (s, 2H, CH₂), 6.50 (s, 1H), 7.25 (m, 1H), 7.60 (m, 1H), 7.95 (m, 1H), 8.05 (m, 1H), 8.10 (m, 1H), 8.65 (m, 1H), 12.20 (brs, 1H) ppm (FIG. 28)

¹H NMR (DMSO-d₆, 400 MHz), δ=0.85 (t, J=7.2 Hz, 3H, CH₃), 1.25-1.50 (m, 8H, 4 CH₂), 1.84 (q, J=7.2 Hz, 2H, CH₂), 2.95 (t, J=6.9 Hz, 2H, CH₂), 3.13 (t, J=6.9 Hz, 2H, CH₂), 3.55 (m, 14H, 7 OCH₂), 4.05 (m, 2H, OCH₂), 5.25 (m, 2H, CH₂), 5.44 (s, 2H, CH₂), 6.55 (s, 1H), 7.26 (m, 1H), 7.60 (m, 1H), 7.95 (m, 1H), 8.08 (m, 1H), 8.16 (m, 1H), 8.67 (m, 1H), 12.10 (brs, 1H) ppm.

¹³C NMR (DMSO-d₆, 100 MHz), δ=7.76 (CH₃), 25.94, 29.14, 29.34, 30.21, 34.71, 50.19, 64.90, 65.24, 66.20, 68.88, 69.75 (m, 9 OCH₂), 72.36, 96.64 (CH), 118.59 (CH), 118.97, 126.19 (CH), 128.40, 130.21 (CH), 130.84, 131.09 (CH), 131.64, 145.44, 149.71, 149.99, 152.10, 154.02, 156.15, 156.79, 172.44 (COO), 172.61(COO) ppm (FIG. 29).

LSIMS (Matrix: Thioglycerol), m/e=799 [M⁺+1], 429, 365. HRMS (LSIMS, Matrix: thioglyerol): found 799.34053, required 799.34018 for [C₃₉H₅₁N₄O₁₄]⁺. (FIG. 30).

UV-vis (DMF) λ(ε=283 (10 600), 332 (11 800), 367 (28 900), 385 (25 000) nm. UV-vis (methanol) λ(ε)=283 (15 800), 332 (11 800), 367 (28 900), 385 (25 000) nm. UV-vis (DMSO) λ(ε)=286 (17 100), 332 (9 300), 368 (23 700), 386 (21 100) nm. (FIG. 32). Anal. Calcd. for C₃₉H₅₀N₄O₁₄.0.33H₂O: C, 58.20; H, 6.35; N, 6.96. Found: C, 57 83; H, 6.05; N, 7.05.
Synthesis of SYN-027

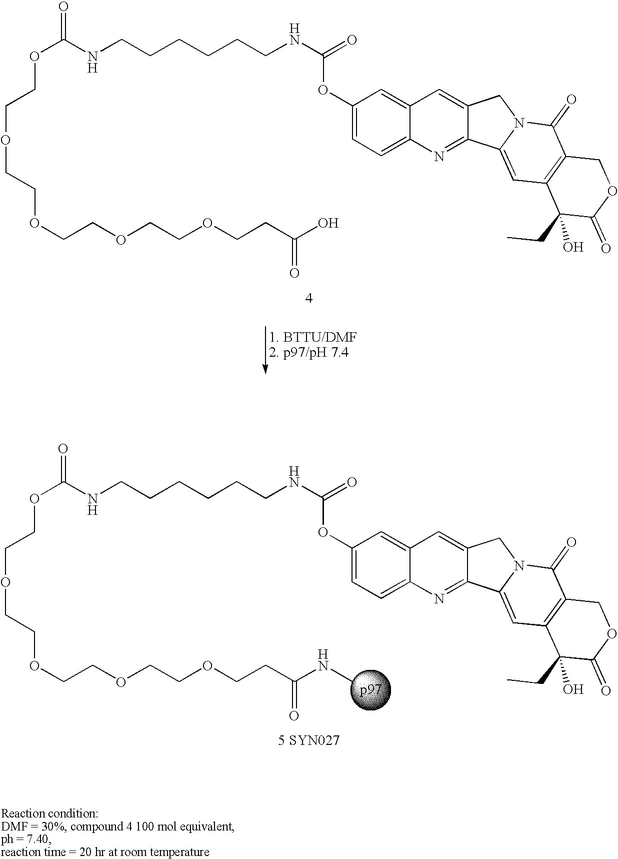

A mixture of propoic acid-PEG4-carbamato-hexyl-carbamato-10-hydroxycamptothecin [4, 114.5 mg, 0.14 mmol], BTTU (138 mg, 0.43 mmol, 3.0 equivalent of 4), triethylamine (0.208 mL, 1.434 mmol, 10 equivalent of 4), DMF (10 mL) is stirred at room temperature for 60 min. The solution is saved for the following reaction.

P97 (lot# 19, O.D. at 280 nm=1.50, c=1.23 mg/mL, 110 mL, 1.433×10⁻³ mM) is placed in a 250 mL round-bottomed flask. To this solution, camptothecin compound prepared from the above (10 mL, 0.14 mmol, 100 equiv., mixed with 37 mL DMF, ˜30% for the whole solution) is added dropwise over a period of 5 min under vigorously stirring. A few drops of NaOH (1M) is added to adjust pH=7.40. The mixture is stirred at room temperature for 20 hr, then purified by dialysis using snake skin tube (WMCO=10K) against PBS (10 mM, pH=7.4). MSR=5.5, protein recovery 85%, the final volume is 154 mL.

SYN-027 (10CPT-PEG4-p97) Method 1

| | | | 10-OH-CPT | | |
|---|---|---|---|---|---|
| Sample | 280 nm | 382 nm | (mg/mL) | P97 (mg/mL) | MSR |
| SYN027 | 1.2222 | 0.8425 | 0.015481 | 0.749631 | 5.36 |

SYN-027 (10CPT-PEG4-p97) Method 2

| Abs | | | 10-OH-CPT | | |
|---|---|---|---|---|---|
| Sample | 280 nm | 382 nm | (mg/mL) | P97 (mg/mL) | |
| A | 1.2222 | 0.8425 | 0.015808 | 0.746375 | 5.49 |

### Reaction of SN-38 with 1,6-diisocyanatohexane

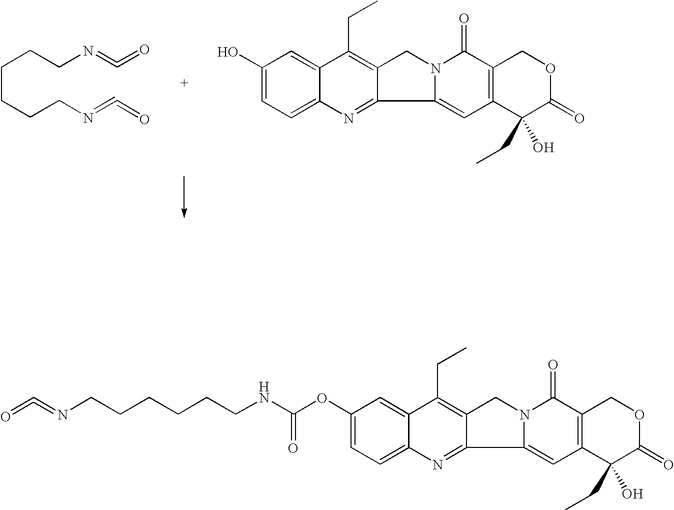

7-Ethyl-10-hydroxycamptothecin (SN-38, 600 mg, 1.53 mmol) and anhydrous DMF (100 mL) was added to a 250-mL single-necked round-bottomed flask, equipped with a magnetic stirrer bar. The flask is immersed in an ultrasonic bath until all solid is dissolved. The mixture is stirred and 1,6-bis(isocyanate)hexane (2.57 mL, 2.57 g, 15.3 mmol, 10.0 equiv.) was added, followed by triethylamine (2 mL, 14 mmol, 9.0 equiv.). The flask was wrapped with alumni foil to protect from light. The reaction was monitored by TLC (dichloromethane/methanol, 95/5, V/V). The starting material has R_{f} 0.5, and the product R_{f} is 0.8. After 20 min, TLC confirms that the reaction is finished. The solvent is removed under vacuum till dry. The residue is then mixed with anhydrous ether (80 mL) and then the flask is placed in the ultrasonic bath for 30 s. The suspension is then kept for 3 h at 4° C. The solid is collected by suction filtration to yield the expected product (797 mg, 93%) as light yellow powder.

M.p. 130-135° C. (dec.)

IR, ν=3418, 2933, 2860, 2272, 1722, 1656, 1602, 1560, 1460, 1191, 1165, 1109, 1033, 997, 920, 837 cm-1. (FIG. 36)

¹H NMR (DMSO-d₆, 400 MHz), δ=0.87 (t, J=7.0 Hz, 3H, CH₃), 1.30 (m, 11H, CH₃, 4 CH₂), 1.55 (m, 2H, CH₂), 1.85 (m, 2H, CH₂), 2.95 (m, 2H, CH₂), 3.25 (m, 4H, 2CH₂), 5.30 (d, J=11.8 Hz, 2H, CH₂), 5.40 (s, 2H, CH₂), 5.70 (m, 1H, NH), 6.50 (s, 1H, OH), 7.30 (s, 1H), 7.60 (s, 1H), 7.90 (m, 2H), 8.10 (t, J=9.3 Hz, 1H) ppm. (FIG. 33)

¹³C-APT NMR (DMSO-d₆, 100 MHz), δ=7.77 (CH₃), 13.73 (CH₃), 22.20, 25.27, 26.01, 29.03, 30.45, 39.91, 40.50, 42.48, 49.49, 65.24, 72.35, 96.49 (CH), 114.52 (CH), 118.83, 125.81 (CH), 127.04, 128.37, 130.97 (CH), 145.01, 146.08, 149.85, 150.00, 151.46, 151.51, 154.08, 156.79, 158.09, 172.43 (COO) ppm. (FIG. 34).

LSIMS (Matrix: glycerol), m/e=561 [M⁺+1], 393, 349. HRMS (LSIMS, Matrix: glycerol): found 561.23418, required 561.23410 for [C₃₀H₃₃N₄O₇]⁺. (FIG. 35).

UV-Vis (DMF) λ(ε)=380 (18 400), 365 (20 600), 335 (10 700), 290 (10400), 270 (11 000) nm. UV-Vis (DMSO) λ(ε=380(18800), 365(20600), 335(10200), 295(7100), 265 (12 700) nm. UV-Vis (MeOH) λ(ε)=360 (20 700), 255 (16 600) nm. UV-Vis (20% DMF/80% PBS) λ(ε)=370 (17 900), 360 (18 300), 260 (16 200) nm. (FIG. 36).

Anal. Calcd. for C₃₀H₃₂N₄O7.0.5H₂O: C, 63.26; H, 5.84; N, 9.84. Found: C, 63.55; H, 6.05; N, 9.95.

FPLC, NMR, MSR determinations and protein recovery data-obtained from the above methods are depicted in FIGS. 14-36.

All publications, patents, and patent applications and references cited in this specification are herein incorporated by reference to the extent not inconsistent with the present disclosure as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of forming a bioconjugate compound of the formula: 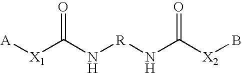
wherein:
A is an active agent moiety;
B is a biopolymer moiety;
X₁ and X₂ are independently N or O; and
R is substituted alkylene or unsubstituted alkylene or unsubstituted or substituted heteroalkylene, wherein said R moiety is from 1 to about 30 atoms in length;
said method comprising:
contacting one of an A precursor or a B precursor, wherein the A precursor and the B precursor independently comprise an active hydroxy or amino functionality, with a bifunctional isocyanate compound of one of the following formulae: 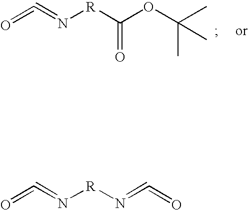
under reaction conditions wherein the isocyanate functional group covalently reacts with the hydroxy or the amino group of the A precursor or the B precursor to form a first reaction product; and then
contacting the other of A precursor or the B precursor with the first reaction product under reaction conditions wherein the first reaction product reacts with the other of the A precursor or the B precursor to form the bioconjugate compound.

2. A method according to claim 1, wherein said active agent is a drug.

3. A method according to claim 1, wherein said biopolymer is a protein.

4. A method according to claim 1, wherein A is contacted with the bifunctional isocyanate moiety to form the first reaction product.

5. A method according to claim 1, wherein B is contacted with the bifunctional isocyanate moiety to form the first reaction product.

6. A method according to claim 1, wherein X₁ is O.

7. A method according to claim 1, wherein X₁ is N.

8. A method according to claim 1, wherein X₂ is O.

9. A method according to claim 1, wherein X₂ is N.

10. A method according to claim 1, wherein R is from 1 to about 10 atoms in length.

11. A method according to claim 1, wherein R is —(CH₂O)ₙCH₂— and n is from 1 to about 15.

12. A method according to claim 1, wherein R is —(CH₂CH₂O)ₙCH₂— and n is from 1 to about 10.

13. A method according to claim 1, wherein R is from 3-7 atoms in length.

14. A method according to claim 1, wherein the bifunctional isocyanate compound is a compound of the formula:

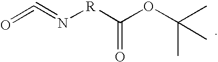

15. A method according to claim 1, wherein the bifunctional isocyanate compound is a compound of the formula:

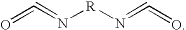

16. A method according to claim 1, wherein the biopolymer is p97.

17. A method according to claim 2, wherein the p97 is recombinant.

18. A method according to claim 1, wherein the active agent is a drug.

19. A method according to claim 1, wherein the drug is an antineoplastic agent.

20. A method according to claim 18, wherein the drug is camptothecin, 10-hydroxycamptothecin, taxol, or doxorubicin.

21. A method according to claim 18, wherein the drug is useful for treating a disorder of the central nervous system.

22. A method according to claim 1, wherein the active agent is an enzyme.

23. A method according to claim 16, wherein the active agent is an enzyme.

24. A method according to claim 18, wherein the drug is a drug of less than 1000 daltons.

25. A method according to claim 18, wherein the drug is a drug of less than 400 daltons.

26. A bioconjugate compound of the formula: 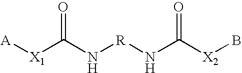
wherein:
A is an active agent moiety;
B is a biopolymer moiety;
X₁ and X₂ are independently N or O; and
R is substituted alkylene or unsubstituted alkylene or unsubstituted or substituted heteroalkylene from 1 to about 30 atoms in length.

27. A compound according to claim 26, wherein said active agent is labeled.

28. A compound according to claim 26, wherein said biopolymer is a protein.

29. A compound according to claim 26, wherein X₁ is O.

30. A compound according to claim 26, wherein X₁ is N.

31. A compound according to claim 26, wherein X₂ is O.

32. A compound according to claim 26, wherein X₂ is N.

33. A compound according to claim 26, wherein R is from 1 to about 10 atoms in length.

34. A compound according to claim 26, wherein R is —(CH₂O)ₙCH₂— and n is from 1 to about 15.

35. A compound according to claim 26, wherein R is —(CH₂CH₂O)ₙCH₂— and n is from 1 to about 10.

36. A compound according to claim 26, wherein R is from 3-7 atoms in length.

37. A compound according to claim 26, wherein the biopolymer is p97.

38. A compound according to claim 26, wherein the active agent is a drug.

39. A compound according to claim 26, wherein the drug is an antineoplastic agent.

40. A compound according to claim 39, wherein the drug is camptothecin, 10-hydroxycaptothecin, taxol, or doxorubicin.

41. A compound according to claim 38, wherein the drug is useful for treating a disorder of the central nervous system.

42. A compound according to claim 26, wherein the active agent is an enzyme.

43. A compound according to claim 37, wherein the active agent is aldurazyme.

44. A compound according to claim 38, wherein the drug is a drug of less than 1000 daltons.

45. A compound according to claim 38, wherein the drug is a drug of less than 400 daltons.

46. A composition comprising a compound of any one of claims 26-45 and a pharmacologically acceptable excipient.

47. A method of treating a subject, said method comprising administering to said subject a composition according to claim 46.

48. A method according to claim 47, wherein said administering is by an intravenous, oral, intraperitoneal, dermal or rectal route.

49. A compound according to claim 26, wherein the bioconjugate further comprises a label.

50. A compound according to claim 26, wherein the bioconjugate is SYN027.

51. A bioconjugate, comprising p97 covalently linked via an amide moiety to a PEG chain of up to 50 atoms in length which is in turn coupled via a carbamate moiety to an alkyl chain of from 1-15 atoms in length which is in turn linked via a carbamate moiety to a first active agent.

52. A bioconjugate according to claim 51, wherein PEG is PEG4-10.

53. A bioconjugate according to claim 51, wherein the alkyl is homoalkyl from 3 to 10 atoms long.

54. A bioconjugate according to claim 51, wherein the first active agent is 10-hydroxycamptothecin.

55. A bioconjugate according to claim 51, wherein the bioconjugate comprises at least one other covalent linkage via an amide moiety to a PEG chain of up to 50 atoms in length which is in turn coupled via a carbamate moiety to an alkyl chain of from 1-15 atoms in length which is in turn linked via a carbamate moiety to a second active agent.

56. A bioconjugate according to claim 55, wherein the first and second active agents are the same or different, and if different, are useful in treating the same disease or condition.
